SEQUENCE LISTING (108 sequences)

SEQ ID NO: 1 - DNA, 129 bp, synthetic construct
  misc_feature 1..129: note = cgRNA A
  source 1..129: mol_type = other DNA
aactttcagtttagcggtctgttttagagctagaaatagcaagttaaaataaggctagtccgatcaaacgggtaaacaaacaggataattaaggaggcagtacccgggcaccgagtcggtgcttttttt

SEQ ID NO: 2 - DNA, 129 bp, synthetic construct
  misc_feature 1..129: note = cgRNA B
  source 1..129: mol_type = other DNA
aactttcagtttagcggtctgttttagagctagaaatagcaagttaaaataaggctagtccgtatcatggggttgtgtgttgttgtaagtgtgtgtgtgttgccccggcaccgagtcggtgcttttttt

SEQ ID NO: 3 - DNA, 129 bp, synthetic construct
  misc_feature 1..129: note = cgRNA C
  source 1..129: mol_type = other DNA
aactttcagtttagcggtctgttttagagctagaaatagcaagttaaaataaggctagtccgaatataggggaagagaaagaagaagagaagagaaagatgtccccggcaccgagtcggtgcttttttt

SEQ ID NO: 4 - DNA, 40 bp, synthetic construct
  misc_feature 1..40: note = Trigger XA
  source 1..40: mol_type = other DNA
tactgcctccttaattatcctgtttgtttacccgtttgat

SEQ ID NO: 5 - DNA, 40 bp, synthetic construct
  misc_feature 1..40: note = Trigger XB
  source 1..40: mol_type = other DNA
caacacacacacacttacaacaacacacaaccccatgata

SEQ ID NO: 6 - DNA, 40 bp, synthetic construct
  misc_feature 1..40: note = Trigger XC
  source 1..40: mol_type = other DNA
acatctttctcttctcttcttctttctcttcccctatatt

SEQ ID NO: 7 - DNA, 165 bp, synthetic construct
  misc_feature 1..165: note = cgRNA A
  source 1..165: mol_type = other DNA
catctaattcaacaagaattgttttagagctacaccttacgccggttcaattccaagtcccttccagtagcaagttaaaataaggctagtccgttatcaacttaacaccctttacaaaccttcctcttcctttaccctaagtggcaccgagtcggtgcttttttt

SEQ ID NO: 8 - DNA, 165 bp, synthetic construct
  misc_feature 1..165: note = cgRNA B
  source 1..165: mol_type = other DNA
catctaattcaacaagaattgttttagagctagtaatcgaatcatagtaaatttcccatcgtcataatagcaagttaaaataaggctagtccgttatcaacttcatacgggtctgaagtagttcattcttatacagtcaagtggcaccgagtcggtgcttttttt

SEQ ID NO: 9 - DNA, 165 bp, synthetic construct
  misc_feature 1..165: note = cgRNA C
  source 1..165: mol_type = other DNA
catctaattcaacaagaattgttttagagctagtcgttaccttatcaatatcaacctccgcatacactagcaagttaaaataaggctagtccgttatcaacttgcacataggacccaacatgccaacagagaagagttaagtggcaccgagtcggtgcttttttt

SEQ ID NO: 10 - DNA, 70 bp, synthetic construct
  misc_feature 1..70: note = Trigger XA
  source 1..70: mol_type = other DNA
agggtaaaggaagaggaaggtttgtaaagggtgttctggaagggacttggaattgaaccggcgtaaggtg

SEQ ID NO: 11 - DNA, 70 bp, synthetic construct
  misc_feature 1..70: note = Trigger XB
  source 1..70: mol_type = other DNA
gactgtataagaatgaactacttcagacccgtatgttatgacgatgggaaatttactatgattcgattac

SEQ ID NO: 12 - DNA, 70 bp, synthetic construct
  misc_feature 1..70: note = Trigger XC
  source 1..70: mol_type = other DNA
aactcttctctgttggcatgttgggtcctatgtgcgtgtatgcggaggttgatattgataaggtaacgac

SEQ ID NO: 13 - DNA, 129 bp, synthetic construct
  misc_feature 1..129: note = cgRNA Q
  source 1..129: mol_type = other DNA
gagtcgcgtgtagcgaagcagttttagagctagaaatagcaagttaaaataaggctagtccgatctttgcgcgttagtttcgttcgtatttctgtcatgtttgcgcggcaccgagtcggtgcttttttt

SEQ ID NO: 14 - DNA, 129 bp, synthetic construct
  misc_feature 1..129: note = cgRNA R
  source 1..129: mol_type = other DNA
gagtcgcgtgtagcgaagcagttttagagctagaaatagcaagttaaaataaggctagtccgtatcgccgggttcaagcagatgtggcatttcagtgtagttcccgggcaccgagtcggtgcttttttt

SEQ ID NO: 15 - DNA, 129 bp, synthetic construct
  misc_feature 1..129: note = cgRNA S
  source 1..129: mol_type = other DNA
gagtcgcgtgtagcgaagcagttttagagctagaaatagcaagttaaaataaggctagtccgtccattcgggtttactattacaatcttacgtgttctcattcccgggcaccgagtcggtgcttttttt

SEQ ID NO: 16 - DNA, 129 bp, synthetic construct
  misc_feature 1..129: note = cgRNA T
  source 1..129: mol_type = other DNA
gagtcgcgtgtagcgaagcagttttagagctagaaatagcaagttaaaataaggctagtccggataaagggaaagatgaagtgatgtgaagatagagttggatcccggcaccgagtcggtgcttttttt

SEQ ID NO: 17 - DNA, 117 bp, synthetic construct
  misc_feature 1..117: note = Trigger XQ
  source 1..117: mol_type = other DNA
agtcaggccacttgtgccacggtttgagcaaaccgtgctgcctgtagctccgccaataatgggaggcgtaaacatgacagaaatacgaacgaaactaacgcgcaaagatcttttttt

SEQ ID NO: 18 - DNA, 117 bp, synthetic construct
  misc_feature 1..117: note = Trigger XR
  source 1..117: mol_type = other DNA
agtcaggccacttgtgccacggtttgagcaaaccgtgctgcctgtagctccgccaataatgggaggcgtaactacactgaaatgccacatctgcttgaacccggcgatacttttttt

SEQ ID NO: 19 - DNA, 117 bp, synthetic construct
  misc_feature 1..117: note = Trigger XS
  source 1..117: mol_type = other DNA
agtcaggccacttgtgccacggtttgagcaaaccgtgctgcctgtagctccgccaataatgggaggcgtaatgagaacacgtaagattgtaatagtaaacccgaatggacttttttt

SEQ ID NO: 20 - DNA, 117 bp, synthetic construct
  misc_feature 1..117: note = Trigger XT
  source 1..117: mol_type = other DNA
agtcaggccacttgtgccacggtttgagcaaaccgtgctgcctgtagctccgccaataatgggaggcgttccaactctatcttcacatcacttcatctttccctttatccttttttt

SEQ ID NO: 21 - DNA, 117 bp, synthetic construct; identical to SEQ ID NO: 17
  misc_feature 1..117: note = Trigger XQ - 40 nt
  source 1..117: mol_type = other DNA

SEQ ID NO: 22 - DNA, 127 bp, synthetic construct
  misc_feature 1..127: note = 50 nt Trigger - Center
  source 1..127: mol_type = other DNA
agtcaggccacttgtgccacggtttgagcaaaccgtgctgcctgtagctccgccaataatgggaggcgtgccgaaaacatgacagaaatacgaacgaaactaacgcgcaaagattccagcttttttt

SEQ ID NO: 23 - DNA, 127 bp, synthetic construct
  misc_feature 1..127: note = 50 nt Trigger - 40 nt XQ at 3'
  source 1..127: mol_type = other DNA
agtcaggccacttgtgccacggtttgagcaaaccgtgctgcctgtagctccgccaataatgggaggcgtgccgatccagaaacatgacagaaatacgaacgaaactaacgcgcaaagatcttttttt

SEQ ID NO: 24 - DNA, 127 bp, synthetic construct
  misc_feature 1..127: note = 50 nt Trigger - 40 nt XQ at 5'
  source 1..127: mol_type = other DNA
agtcaggccacttgtgccacggtttgagcaaaccgtgctgcctgtagctccgccaataatgggaggcgtaaacatgacagaaatacgaacgaaactaacgcgcaaagatgccgatccagcttttttt

SEQ ID NO: 25 - DNA, 147 bp, synthetic construct
  misc_feature 1..147: note = 70 nt Trigger - Center
  source 1..147: mol_type = other DNA
agtcaggccacttgtgccacggtttgagcaaaccgtgctgcctgtagctccgccaataatgggaggcgtgccgagccgagccgaaaacatgacagaaatacgaacgaaactaacgcgcaaagatgccgagccgatccagcttttttt

SEQ ID NO: 26 - DNA, 147 bp, synthetic construct
  misc_feature 1..147: note = 70 nt Trigger - 40 nt XQ at 3'
  source 1..147: mol_type = other DNA
agtcaggccacttgtgccacggtttgagcaaaccgtgctgcctgtagctccgccaataatgggaggcgtgccgatccaggccgatccaggccgatccagaaacatgacagaaatacgaacgaaactaacgcgcaaagatcttttttt

SEQ ID NO: 27 - DNA, 147 bp, synthetic construct
  misc_feature 1..147: note = 70 nt Trigger - 40 nt XQ at 5'
  source 1..147: mol_type = other DNA
agtcaggccacttgtgccacggtttgagcaaaccgtgctgcctgtagctccgccaataatgggaggcgtaaacatgacagaaatacgaacgaaactaacgcgcaaagatgccgatccaggccgatccaggccgatccagcttttttt

SEQ ID NO: 28 - DNA, 177 bp, synthetic construct
  misc_feature 1..177: note = 100 nt Trigger - Center
  source 1..177: mol_type = other DNA
agtcaggccacttgtgccacggtttgagcaaaccgtgctgcctgtagctccgccaataatgggaggcgtgccgatccaggccgatccaggccgatccagaaacatgacagaaatacgaacgaaactaacgcgcaaagatgccgatccaggccgatccaggccgatccagcttttttt

SEQ ID NO: 29 - DNA, 177 bp, synthetic construct
  misc_feature 1..177: note = 100 nt Trigger - 40 nt XQ at 3'
  source 1..177: mol_type = other DNA
agtcaggccacttgtgccacggtttgagcaaaccgtgctgcctgtagctccgccaataatgggaggcgtgccgatccaggccgatccaggccgatccaggccgatccaggccgatccaggccgatccagaaacatgacagaaatacgaacgaaactaacgcgcaaagatcttttttt

SEQ ID NO: 30 - DNA, 177 bp, synthetic construct
  misc_feature 1..177: note = 100 nt Trigger - 40 nt XQ at 5'
  source 1..177: mol_type = other DNA
agtcaggccacttgtgccacggtttgagcaaaccgtgctgcctgtagctccgccaataatgggaggcgtaaacatgacagaaatacgaacgaaactaacgcgcaaagatgccgatccaggccgatccaggccgatccaggccgatccaggccgatccaggccgatccagcttttttt

SEQ ID NO: 31 - DNA, 227 bp, synthetic construct
  misc_feature 1..227: note = 150 nt Trigger - Center
  source 1..227: mol_type = other DNA
agtcaggccacttgtgccacggtttgagcaaaccgtgctgcctgtagctccgccaataatgggaggcgtgccgatccaggccgatccaggccgagccgatccaggccgatccaggccgatccagaaacatgacagaaatacgaacgaaactaacgcgcaaagatgccgatccaggccgatccaggccgagccgatccaggccgatccaggccgatccagcttttttt

SEQ ID NO: 32 - DNA, 227 bp, synthetic construct
  misc_feature 1..227: note = 150 nt Trigger - 40 nt XQ at 3'
  source 1..227: mol_type = other DNA
agtcaggccacttgtgccacggtttgagcaaaccgtgctgcctgtagctccgccaataatgggaggcgtgccgatccaggccgatccaggccgatccaggccgatccaggccgatccaggccgatccaggccgatccaggccgatccaggccgatccaggccgatccaggccgatccagaaacatgacagaaatacgaacgaaactaacgcgcaaagatcttttttt

SEQ ID NO: 33 - DNA, 227 bp, synthetic construct
  misc_feature 1..227: note = 150 nt Trigger - 40 nt XQ at 5'
  source 1..227: mol_type = other DNA
agtcaggccacttgtgccacggtttgagcaaaccgtgctgcctgtagctccgccaataatgggaggcgtaaacatgacagaaatacgaacgaaactaacgcgcaaagatgccgatccaggccgatccaggccgatccaggccgatccaggccgatccaggccgatccaggccgatccaggccgatccaggccgatccaggccgatccaggccgatccagcttttttt

SEQ ID NO: 34 - DNA, 90 bp, synthetic construct
  misc_feature 1..90: note = cgRNA M
  source 1..90: mol_type = other DNA
gagtcgcgtgtagcgaagcagtttaagagctatgctggaaacagcatagcaagtttaaataaggctagtccgttatcagcaccttttttt

SEQ ID NO: 35 - DNA, 90 bp, synthetic construct
  misc_feature 1..90: note = cgRNA N
  source 1..90: mol_type = other DNA
gagtcgcgtgtagcgaagcagtttaagagctatgctggaaacagcatagcaagtttaaataaggctagtccgttatcaggcccttttttt

SEQ ID NO: 36 - DNA, 90 bp, synthetic construct
  misc_feature 1..90: note = cgRNA O
  source 1..90: mol_type = other DNA
gagtcgcgtgtagcgaagcagtttaagagctatgctggaaacagcatagcaagtttaaataaggctagtccgttatcacccacttttttt

SEQ ID NO: 37 - DNA, 27 bp, synthetic construct
  misc_feature 1..27: note = Trigger XM
  source 1..27: mol_type = other DNA
gtgcggcaccgagtcggtgcttttttt

SEQ ID NO: 38 - DNA, 27 bp, synthetic construct
  misc_feature 1..27: note = Trigger XN
  source 1..27: mol_type = other DNA
ggccggcaccgagtcggtgcttttttt

SEQ ID NO: 39 - DNA, 27 bp, synthetic construct
  misc_feature 1..27: note = Trigger XO
  source 1..27: mol_type = other DNA
tgggggcaccgagtcggtgcttttttt

SEQ ID NO: 40 - DNA, 96 bp, synthetic construct
  misc_feature 1..96: note = cgRNA M
  source 1..96: mol_type = other DNA
gagtcgcgtgtagcgaagcagtttaagagctatgctggaaacagcatagcaagtttaaataaggctagtccgttatcagcacatcccacttttttt

SEQ ID NO: 41 - DNA, 96 bp, synthetic construct
  misc_feature 1..96: note = cgRNA N
  source 1..96: mol_type = other DNA
gagtcgcgtgtagcgaagcagtttaagagctatgctggaaacagcatagcaagtttaaataaggctagtccgttatcaggccaggttccttttttt

SEQ ID NO: 42 - DNA, 96 bp, synthetic construct
  misc_feature 1..96: note = cgRNA O
  source 1..96: mol_type = other DNA
gagtcgcgtgtagcgaagcagtttaagagctatgctggaaacagcatagcaagtttaaataaggctagtccgttatcacccagaacaccttttttt

SEQ ID NO: 43 - DNA, 102 bp, synthetic construct
  misc_feature 1..102: note = Trigger XM
  source 1..102: mol_type = other DNA
agtcaggccacttgtgccacggtttgagcaaaccgtgctgcctgtagctccgccaataatgggaggcgttgggatgtgcggcaccgagtcggtgcttttttt

SEQ ID NO: 44 - DNA, 102 bp, synthetic construct
  misc_feature 1..102: note = Trigger XN
  source 1..102: mol_type = other DNA
agtcaggccacttgtgccacggtttgagcaaaccgtgctgcctgtagctccgccaataatgggaggcgtgaacctggccggcaccgagtcggtgcttttttt

SEQ ID NO: 45 - DNA, 102 bp, synthetic construct
  misc_feature 1..102: note = Trigger XO
  source 1..102: mol_type = other DNA
agtcaggccacttgtgccacggtttgagcaaaccgtgctgcctgtagctccgccaataatgggaggcgtgtgttctgggggcaccgagtcggtgcttttttt

SEQ ID NO: 46 - DNA, 96 bp, synthetic construct; identical to SEQ ID NO: 40
  misc_feature 1..96: note = cgRNA M
  source 1..96: mol_type = other DNA

SEQ ID NO: 47 - DNA, 96 bp, synthetic construct; identical to SEQ ID NO: 41
  misc_feature 1..96: note = cgRNA N
  source 1..96: mol_type = other DNA

SEQ ID NO: 48 - DNA, 96 bp, synthetic construct; identical to SEQ ID NO: 42
  misc_feature 1..96: note = cgRNA O
  source 1..96: mol_type = other DNA

SEQ ID NO: 49 - DNA, 102 bp, synthetic construct; identical to SEQ ID NO: 43
  misc_feature 1..102: note = Trigger XM
  source 1..102: mol_type = other DNA

SEQ ID NO: 50 - DNA, 102 bp, synthetic construct; identical to SEQ ID NO: 44
  misc_feature 1..102: note = Trigger XN
  source 1..102: mol_type = other DNA

SEQ ID NO: 51 - DNA, 102 bp, synthetic construct; identical to SEQ ID NO: 45
  misc_feature 1..102: note = Trigger XO
  source 1..102: mol_type = other DNA

SEQ ID NO: 52 - DNA, 94 bp, synthetic construct
  misc_feature 1..94: note = cgRNA M
  source 1..94: mol_type = other DNA
gagtcgcgtgtagcgaagcagtttaagagctatgctggaaacagcatagcaagtttaaataaggctagtccgttatcagcacatcccttttttt

SEQ ID NO: 53 - DNA, 94 bp, synthetic construct
  misc_feature 1..94: note = cgRNA N
  source 1..94: mol_type = other DNA
gagtcgcgtgtagcgaagcagtttaagagctatgctggaaacagcatagcaagtttaaataaggctagtccgttatcaggccaggtcttttttt

SEQ ID NO: 54 - DNA, 94 bp, synthetic construct
  misc_feature 1..94: note = cgRNA O
  source 1..94: mol_type = other DNA
gagtcgcgtgtagcgaagcagtttaagagctatgctggaaacagcatagcaagtttaaataaggctagtccgttatcacccagaaccttttttt

SEQ ID NO: 55 - DNA, 100 bp, synthetic construct
  misc_feature 1..100: note = Trigger XM
  source 1..100: mol_type = other DNA
agtcaggccacttgtgccacggtttgagcaaaccgtgctgcctgtagctccgccaataatgggaggcgtggatgtgcggcaccgagtcggtgcttttttt

SEQ ID NO: 56 - DNA, 100 bp, synthetic construct
  misc_feature 1..100: note = Trigger XN
  source 1..100: mol_type = other DNA
agtcaggccacttgtgccacggtttgagcaaaccgtgctgcctgtagctccgccaataatgggaggcgtacctggccggcaccgagtcggtgcttttttt

SEQ ID NO: 57 - DNA, 100 bp, synthetic construct
  misc_feature 1..100: note = Trigger XO
  source 1..100: mol_type = other DNA
agtcaggccacttgtgccacggtttgagcaaaccgtgctgcctgtagctccgccaataatgggaggcgtgttctgggggcaccgagtcggtgcttttttt

SEQ ID NO: 58 - DNA, 92 bp, synthetic construct
  misc_feature 1..92: note = cgRNA M
  source 1..92: mol_type = other DNA
gagtcgcgtgtagcgaagcagtttaagagctatgctggaaacagcatagcaagtttaaataaggctagtccgttatcagcacatcttttttt

SEQ ID NO: 59 - DNA, 92 bp, synthetic construct
  misc_feature 1..92: note = cgRNA N
  source 1..92: mol_type = other DNA
gagtcgcgtgtagcgaagcagtttaagagctatgctggaaacagcatagcaagtttaaataaggctagtccgttatcaggccagcttttttt

SEQ ID NO: 60 - DNA, 92 bp, synthetic construct
  misc_feature 1..92: note = cgRNA O
  source 1..92: mol_type = other DNA
gagtcgcgtgtagcgaagcagtttaagagctatgctggaaacagcatagcaagtttaaataaggctagtccgttatcacccagacttttttt

SEQ ID NO: 61 - DNA, 98 bp, synthetic construct
  misc_feature 1..98: note = Trigger XM
  source 1..98: mol_type = other DNA
agtcaggccacttgtgccacggtttgagcaaaccgtgctgcctgtagctccgccaataatgggaggcgtatgtgcggcaccgagtcggtgcttttttt

SEQ ID NO: 62 - DNA, 98 bp, synthetic construct
  misc_feature 1..98: note = Trigger XN
  source 1..98: mol_type = other DNA
agtcaggccacttgtgccacggtttgagcaaaccgtgctgcctgtagctccgccaataatgggaggcgtctggccggcaccgagtcggtgcttttttt

SEQ ID NO: 63 - DNA, 98 bp, synthetic construct
  misc_feature 1..98: note = Trigger XO
  source 1..98: mol_type = other DNA
agtcaggccacttgtgccacggtttgagcaaaccgtgctgcctgtagctccgccaataatgggaggcgttctgggggcaccgagtcggtgcttttttt

SEQ ID NO: 64 - DNA, 125 bp, synthetic construct
  misc_feature 1..125: note = cgRNA 40
  source 1..125: mol_type = other DNA
agtcgcgtgtagcgaagcagtttaagagctatgctggaaacagcatagcaagtttaaataaggctagtccgttatcacatatcccatccacctccacctccacctccacattcccaccttttttt

SEQ ID NO: 65 - DNA, 115 bp, synthetic construct
  misc_feature 1..115: note = cgRNA 30
  source 1..115: mol_type = other DNA
agtcgcgtgtagcgaagcagtttaagagctatgctggaaacagcatagcaagtttaaataaggctagtccgttatcacatatcccatccacctccacctccacctcccttttttt

SEQ ID NO: 66 - DNA, 105 bp, synthetic construct
  misc_feature 1..105: note = cgRNA 20
  source 1..105: mol_type = other DNA
agtcgcgtgtagcgaagcagtttaagagctatgctggaaacagcatagcaagtttaaataaggctagtccgttatcacatatcccatccacctccaccttttttt

SEQ ID NO: 67 - DNA, 95 bp, synthetic construct
  misc_feature 1..95: note = cgRNA 10
  source 1..95: mol_type = other DNA
agtcgcgtgtagcgaagcagtttaagagctatgctggaaacagcatagcaagtttaaataaggctagtccgttatcacatatcccatcttttttt

SEQ ID NO: 68 - DNA, 132 bp, synthetic construct
  misc_feature 1..132: note = Trigger X40
  source 1..132: mol_type = other DNA
agtcaggccacttgtgccacggtttgagcaaaccgtgctgcctgtagctccgccaataatgggaggcgtgtgggaatgtggaggtggaggtggaggtggatgggatatgggcaccgagtcggtgcttttttt

SEQ ID NO: 69 - DNA, 122 bp, synthetic construct
  misc_feature 1..122: note = Trigger X30
  source 1..122: mol_type = other DNA
agtcaggccacttgtgccacggtttgagcaaaccgtgctgcctgtagctccgccaataatgggaggcgtggaggtggaggtggaggtggatgggatatgggcaccgagtcggtgcttttttt

SEQ ID NO: 70 - DNA, 113 bp, synthetic construct
  misc_feature 1..113: note = Trigger X20
  source 1..113: mol_type = other DNA
agtcaggccacttgtgccacggtttgagcaaaccgtgctgcctgtagctccgccaataatgggaggcgtgtggaggtggatgggatatgggcaccgagtcggtgctttttttt

SEQ ID NO: 71 - DNA, 102 bp, synthetic construct
  misc_feature 1..102: note = Trigger X10
  source 1..102: mol_type = other DNA
agtcaggccacttgtgccacggtttgagcaaaccgtgctgcctgtagctccgccaataatgggaggcgtatgggatatgggcaccgagtcggtgcttttttt

SEQ ID NO: 72 - DNA, 103 bp, synthetic construct
  misc_feature 1..103: note = Standard gRNA
  source 1..103: mol_type = other DNA
gagtcgcgtgtagcgaagcagttttagagctagaaatagcaagttaaaataaggctagtccgttatcaacttgaaaaagtggcaccgagtcggtgcttttttt

SEQ ID NO: 73 - DNA, 83 bp, synthetic construct
  misc_feature 1..83: note = No-target gRNA
  source 1..83: mol_type = other DNA
gttttagagctagaaatagcaagttaaaataaggctagtccgttatcaacttgaaaaagtggcaccgagtcggtgcttttttt

SEQ ID NO: 74 - DNA, 95 bp, synthetic construct
  misc_feature 1..95: note = 8 nt deletion
  source 1..95: mol_type = other DNA
gagtcgcgtgtagcgaagcagttttagagctagaaatagcaagttaaaataaggctagtccgttatcaacttgaaaaagtggcaccgcttttttt

SEQ ID NO: 75 - DNA, 80 bp, synthetic construct
  misc_feature 1..80: note = 23 nt deletion
  source 1..80: mol_type = other DNA
gagtcgcgtgtagcgaagcagttttagagctagaaatagcaagttaaaataaggctagtccgttatcaacttcttttttt

SEQ ID NO: 76 - DNA, 71 bp, synthetic construct
  misc_feature 1..71: note = 32 nt deletion
  source 1..71: mol_type = other DNA
gagtcgcgtgtagcgaagcagttttagagctagaaatagcaagttaaaataaggctagtccgtcttttttt

SEQ ID NO: 77 - DNA, 58 bp, synthetic construct
  misc_feature 1..58: note = 45 nt deletion
  source 1..58: mol_type = other DNA
gagtcgcgtgtagcgaagcagttttagagctagaaatagcaagttaaaatcttttttt

SEQ ID NO: 78 - DNA, 129 bp, synthetic construct; identical to SEQ ID NO: 1
  misc_feature 1..129: note = cgRNAA
  source 1..129: mol_type = other DNA

SEQ ID NO: 79 - DNA, 129 bp, synthetic construct
  misc_feature 1..129: note = cgRNA E
  source 1..129: mol_type = other DNA
aactttcagtttagcggtctgttttagagctagaaatagcaagttaaaataaggctagtccgacacaaggggaaattaacaacacaacacacacaacacaggccccggcaccgagtcggtgcttttttt

SEQ ID NO: 80 - DNA, 1849 bp, synthetic construct
  misc_feature 1..1849: note = Trigger XA
  source 1..1849: mol_type = other DNA
tggctaaagaaagaggagaaaaggtttatggtagcaggtcatgcctctggcagccccgcattcgggaccgcctctcattcgaattgcgaacatgaagagatccacctcgccggctcgatccagccgcatggcgcgcttctggtcgtcagcgaacatgatcatcgcgtcatccaggccagcgccaacgccgcggaatttctgaatctcggaagcgtactcggcgttccgctcgccgagatcgacggcgatctgttgatcaagatcctgccgcatctcgatcccaccgccgaaggcatgccggtcgcggtgcgctgccggatcggcaatccctctacggagtactgcggtctgatgcatcggcctccggaaggcgggctgatcatcgaactcgaacgtgccggcccgtcgatcgatctgtcaggcacgctggcgccggcgctggagcggatccgcacggcgggttcactgcgcgcgctgtgcgatgacaccgtgctgctgtttcagcagtgcaccggctacgaccgggtgatggtgtatcgtttcgatgagcaaggccacggcctggtattctccgagtgccatgtgcctgggctcgaatcctatttcggcaaccgctatccgtcgtcgactgtcccgcagatggcgcggcagctgtacgtgcggcagcgcgtccgcgtgctggtcgacgtcacctatcagccggtgccgctggagccgcggctgtcgccgctgaccgggcgcgatctcgacatgtcgggctgcttcctgcgctcgatgtcgccgtgccatctgcagttcctgaaggacatgggcgtgcgcgccaccctggcggtgtcgctggtggtcggcggcaagctgtggggcctggttgtctgtcaccattatctgccgcgcttcatccgtttcgagctgcgggcgatctgcaaacggctcgccgaaaggatcgcgacgcggatcaccgcgcttgagagcgaattcggtggtggtggttctggtggtggtggttctatgagtgtcaacttagcttcccagttgcgggaagggacgaaaaaatcccactccatggcggagaacgtcggctttgtcaaatgcttcctcaagggcgttgtcgagaaaaattcctaccgtaagctggttggcaatctctactttgtctacagtgccatggaagaggaaatggcaaaatttaaggaccatcccatcctcagccacatttacttccccgaactcaaccgcaaacaaagcctagagcaagacctgcaattctattacggctccaactggcggcaagaagtgaaaatttctgccgctggccaagcctatgtggaccgagtccggcaagtggccgctacggcccctgaattgttggtggcccattcctacacccgttacctgggggatctttccggcggtcaaattctcaagaaaattgcccaaaatgccatgaatctccacgatggtggcacagctttctatgaatttgccgacattgatgacgaaaaggcttttaaaaatacctaccgtcaagctatgaatgatctgcccattgaccaagccaccgccgaacggattgtggatgaagccaatgacgcctttgccatgaacatgaaaatgttcaacgaacttgaaggcaacctgatcaaggcgatcggcattatggtgttcaacagcctcacccgtcgccgcagtcaaggcagcaccgaagttggcctcgccacctccgaaggctagtaaacgtcgactctcgagtgagattgttgacggtaccgtatttttactgcctccttaattatcctgtttgtttacccgtttgatcgcaaaaaaccccgcttcggcggggttttttcgc

SEQ ID NO: 81 - DNA, 1849 bp, synthetic construct
  misc_feature 1..1849: note = Trigger XE
  source 1..1849: mol_type = other DNA
tggctaaagaaagaggagaaaaggtttatggtagcaggtcatgcctctggcagccccgcattcgggaccgcctctcattcgaattgcgaacatgaagagatccacctcgccggctcgatccagccgcatggcgcgcttctggtcgtcagcgaacatgatcatcgcgtcatccaggccagcgccaacgccgcggaatttctgaatctcggaagcgtactcggcgttccgctcgccgagatcgacggcgatctgttgatcaagatcctgccgcatctcgatcccaccgccgaaggcatgccggtcgcggtgcgctgccggatcggcaatccctctacggagtactgcggtctgatgcatcggcctccggaaggcgggctgatcatcgaactcgaacgtgccggcccgtcgatcgatctgtcaggcacgctggcgccggcgctggagcggatccgcacggcgggttcactgcgcgcgctgtgcgatgacaccgtgctgctgtttcagcagtgcaccggctacgaccgggtgatggtgtatcgtttcgatgagcaaggccacggcctggtattctccgagtgccatgtgcctgggctcgaatcctatttcggcaaccgctatccgtcgtcgactgtcccgcagatggcgcggcagctgtacgtgcggcagcgcgtccgcgtgctggtcgacgtcacctatcagccggtgccgctggagccgcggctgtcgccgctgaccgggcgcgatctcgacatgtcgggctgcttcctgcgctcgatgtcgccgtgccatctgcagttcctgaaggacatgggcgtgcgcgccaccctggcggtgtcgctggtggtcggcggcaagctgtggggcctggttgtctgtcaccattatctgccgcgcttcatccgtttcgagctgcgggcgatctgcaaacggctcgccgaaaggatcgcgacgcggatcaccgcgcttgagagcgaattcggtggtggtggttctggtggtggtggttctatgagtgtcaacttagcttcccagttgcgggaagggacgaaaaaatcccactccatggcggagaacgtcggctttgtcaaatgcttcctcaagggcgttgtcgagaaaaattcctaccgtaagctggttggcaatctctactttgtctacagtgccatggaagaggaaatggcaaaatttaaggaccatcccatcctcagccacatttacttccccgaactcaaccgcaaacaaagcctagagcaagacctgcaattctattacggctccaactggcggcaagaagtgaaaatttctgccgctggccaagcctatgtggaccgagtccggcaagtggccgctacggcccctgaattgttggtggcccattcctacacccgttacctgggggatctttccggcggtcaaattctcaagaaaattgcccaaaatgccatgaatctccacgatggtggcacagctttctatgaatttgccgacattgatgacgaaaaggcttttaaaaatacctaccgtcaagctatgaatgatctgcccattgaccaagccaccgccgaacggattgtggatgaagccaatgacgcctttgccatgaacatgaaaatgttcaacgaacttgaaggcaacctgatcaaggcgatcggcattatggtgttcaacagcctcacccgtcgccgcagtcaaggcagcaccgaagttggcctcgccacctccgaaggctagtaaacgtcgactctcgagtgagattgttgacggtaccgtattttcctgtgttgtgtgtgttgtgttgttaatttccccttgtgtcgcaaaaaaccccgcttcggcggggttttttcgc

SEQ ID NO: 82 - DNA, 129 bp, synthetic construct; identical to SEQ ID NO: 15
  misc_feature 1..129: note = cgRNA
  source 1..129: mol_type = other DNA

SEQ ID NO: 83 - DNA, 117 bp, synthetic construct; identical to SEQ ID NO: 17
  misc_feature 1..117: note = Non-cognate trigger (X')
  source 1..117: mol_type = other DNA

SEQ ID NO: 84 - DNA, 117 bp, synthetic construct; identical to SEQ ID NO: 19
  misc_feature 1..117: note = Cognate-trigger (X)
  source 1..117: mol_type = other DNA

SEQ ID NO: 85 - DNA, 96 bp, synthetic construct; identical to SEQ ID NO: 41
  misc_feature 1..96: note = cgRNA
  source 1..96: mol_type = other DNA

SEQ ID NO: 86 - DNA, 102 bp, synthetic construct; identical to SEQ ID NO: 43
  misc_feature 1..102: note = Non-cognate trigger (X')
  source 1..102: mol_type = other DNA

SEQ ID NO: 87 - DNA, 102 bp, synthetic construct; identical to SEQ ID NO: 44
  misc_feature 1..102: note = Cognate trigger (X)
  source 1..102: mol_type = other DNA

SEQ ID NO: 88 - DNA, 72 bp, synthetic construct
  misc_feature 1..72: note = cgRNA M
  source 1..72: mol_type = other DNA
catctaattcaacaagaattgttttagagctagaaatagcaagttaaaataaggctagtccgttatcagcac

SEQ ID NO: 89 - DNA, 72 bp, synthetic construct
  misc_feature 1..72: note = cgRNA N
  source 1..72: mol_type = other DNA
catctaattcaacaagaattgttttagagctagaaatagcaagttaaaataaggctagtccgttatcaggcc

SEQ ID NO: 90 - DNA, 72 bp, synthetic construct
  misc_feature 1..72: note = cgRNA O
  source 1..72: mol_type = other DNA
catctaattcaacaagaattgttttagagctagaaatagcaagttaaaataaggctagtccgttatcaccca

SEQ ID NO: 91 - DNA, 19 bp, synthetic construct
  misc_feature 1..19: note = Trigger XM
  source 1..19: mol_type = other DNA
gtgcggcaccgagtcggtg

SEQ ID NO: 92 - DNA, 19 bp, synthetic construct
  misc_feature 1..19: note = Trigger XN
  source 1..19: mol_type = other DNA
ggccggcaccgagtcggtg

SEQ ID NO: 93 - DNA, 19 bp, synthetic construct
  misc_feature 1..19: note = Trigger XO
  source 1..19: mol_type = other DNA
tgggggcaccgagtcggtg

SEQ ID NO: 94 - DNA, 96 bp, synthetic construct
  misc_feature 1..96: note = cg5
  source 1..96: mol_type = other DNA
gagtcgcgtgtagcgaagcagttttagagctagaaatagcaagttaaaataaggctagtccgtcccatcgtccgtcccatcaatttcccttttttt

SEQ ID NO: 95 - DNA, 43 bp, synthetic construct
  misc_feature 1..43: note = cg3
  source 1..43: mol_type = other DNA
aatataatacgggacggacgggcaccgagtcggtgcttttttt

SEQ ID NO: 96 - DNA, 97 bp, synthetic construct
  misc_feature 1..97: note = Trigger
  source 1..97: mol_type = other DNA
agtcaggccacttgtgccacggtttgagcaaaccgtgctgcctgtagctccgccaataatgggaggcgtcgtccgtcccgtattatattcttttttt

SEQ ID NO: 97 - DNA, 106 bp, synthetic construct
  misc_feature 1..106: note = cg5
  source 1..106: mol_type = other DNA
gagtcgcgtgtagcgaagcagttttagagctagaaatagcaagttaaaataaggctagtccgatatatacaaacacaacacacacacaacaacaaacacttttttt

SEQ ID NO: 98 - DNA, 132 bp, synthetic construct
  misc_feature 1..132: note = cg3
  source 1..132: mol_type = other DNA
agtcaggccacttgtgccacggtttgagcaaaccgtgctgcctgtagctccgccaataatgggaggcgttgtctagaattgtttgttgttgtgtgtgtgttgtgtttgtggcaccgagtcggtgcttttttt

SEQ ID NO: 99 - DNA, 117 bp, synthetic construct
  misc_feature 1..117: note = Trigger
  source 1..117: mol_type = other DNA
agtcaggccacttgtgccacggtttgagcaaaccgtgctgcctgtagctccgccaataatgggaggcgtacaaacacaacacacacacaacaacaaacaattctagacacttttttt

SEQ ID NO: 100 - DNA, 146 bp, synthetic construct
  misc_feature 1..146: note = cg5
  source 1..146: mol_type = other DNA
aactttcagtttagcggtctgttttagagctagaaatagcaagttaaaataaggctagtccgatatatacaaacacaacacacacacaacaacaaacacaacccaaccagagcgcaaaaaaccccgcttcggcggggttttttcgc

SEQ ID NO: 101 - DNA, 67 bp, synthetic construct
  misc_feature 1..67: note = cg3
  source 1..67: mol_type = other DNA
tgtctagaattgtttgttgttgtgtgtgtgttgtgtttgtggcaccgagtcggtgctttttttcgcc

SEQ ID NO: 102 - DNA, 34 bp, synthetic construct
  misc_feature 1..34: note = No-trigger control
  source 1..34: mol_type = other DNA
cgcaaaaaaccccgcttcggcggggttttttcgc

SEQ ID NO: 103 - DNA, 74 bp, synthetic construct
  misc_feature 1..74: note = Trigger X (Mechanism 4B)
  source 1..74: mol_type = other DNA
ggttgggttgtgtttgttgttgtgtgtgtgttgtgtttgtcgcaaaaaaccccgcttcggcggggttttttcgc

SEQ ID NO: 104 - DNA, 74 bp, synthetic construct
  misc_feature 1..74: note = Trigger X (Mechanism 4C)
  source 1..74: mol_type = other DNA
acaaacacaacacacacacaacaacaaacaattctagacacgcaaaaaaccccgcttcggcggggttttttcgc

SEQ ID NO: 105 - DNA, 146 bp, synthetic construct
  misc_feature 1..146: note = cg5
  source 1..146: mol_type = other DNA
ttgtttaaggctatggtgagaactttcagtttagcggtctgttttagagctagaaatagcaagttaaaataaggctagtccgtaaataaaagcccaccctcaccatagagagcgcaaaaaaccccgcttcggcggggttttttcgc

SEQ ID NO: 106 - DNA, 95 bp, synthetic construct
  misc_feature 1..95: note = cg3
  source 1..95: mol_type = other DNA
ccaccctcaccataggtgctatggtgagggtgggctttggcaccgagtcggtgctttttttcgcaaaaaaccccgcttcggcggggttttttcgc

SEQ ID NO: 107 - DNA, 34 bp, synthetic construct; identical to SEQ ID NO: 102
  misc_feature 1..34: note = No-trigger control
  source 1..34: mol_type = other DNA

SEQ ID NO: 108 - DNA, 54 bp, synthetic construct
  misc_feature 1..54: note = Trigger X
  source 1..54: mol_type = other DNA
ctcaccatagccttgaacaacgcaaaaaaccccgcttcggcggggttttttcgc

(19) United States
(12) Patent Application Publication
(10) Pub. No.: US 2025/0346889 A1
(43) Pub. Date: Nov. 13, 2025

(54) Allosteric Conditional Guide RNAs for Cell-Selective Regulation of CRISPR/Cas

(71) Applicant: California Institute of Technology, Pasadena, CA (US)

(72) Inventors: Lisa Hochrein, Pasadena, CA (US); Mikhail H. Hanewich-Hollatz, Pasadena, CA (US); Zhewei Chen, Pasadena, CA (US); Heyun Li, Pasadena, CA (US); Shashank Gandhi, Pasadena, CA (US); Marianne Bronner, Pasadena, CA (US); Niles A. Pierce, Pasadena, CA (US)

(21) Appl. No.: 19/275,540

(22) Filed: Jul. 21, 2025

Related U.S. Application Data
(63) Continuation of application No. 18/537,662, filed on Dec. 12, 2023, now Pat. No. 12,385,040.
Application No. 18/537,662 is a division of application No. 17/584,237, filed on Jan. 25, 2022, now Pat. No. 11,873,485.
(60) Provisional application No. 63/141,865, filed on Jan. 26, 2021.
(60) Provisional application No. 63/181,808, filed on Apr. 29, 2021.

(51) Int. Cl. C12N 15/11; C12N 9/22

(52) U.S. Cl. CPC C12N 15/11; C12N 9/22; C12N 2310/15; C12N 2310/20; C12N 2310/344; C12N 2310/531; C12N 2310/532; C12N 2800/80

(57) ABSTRACT

Programmable guide RNAs (gRNAs) play a central role in the CRISPR revolution sweeping biology and medicine by directing the function of a Cas protein effector to a target gene of choice. To achieve programmable control over regulatory scope, the activity of a conditional guide RNA (cgRNA) depends on the presence or absence of an RNA trigger, allowing for cell-selective regulation of CRISPR/Cas function. Unlike a standard gRNA, a cgRNA is programmable at multiple levels, with the target-binding sequence controlling the target of Cas activity (edit, silence, induce, or bind a gene of choice) and the triggerbinding sequence controlling the scope of Cas activity. cgRNA mechanisms that are allosteric allow for independent design of the target and trigger sequences, providing the flexibility to select the regulatory target and scope independently. Disclosed herein are allosteric cgRNA mechanisms for both ON→OFF logic (conditional inactivation by an RNA trigger) and OFF→ON logic (conditional activation by an RNA trigger). Allosteric cgRNAs enable restriction of CRISPR/Cas function to a desired cell type, tissue, organ, or disease state. Allosteric cgRNAs provide a versatile platform for cell-selective and tissue-selective research tools, biotechnologies, diagnostics, and therapeutics.

### STATEMENT REGARDING FEDERALLY SPONSORED R&D

This invention was made with government support under Grant No. HR0011-17-2-0008 awarded by DARPA, under Grant No. 7000000323 and Grant No. NNX16AO69A awarded by NASA. The government has certain rights in the invention.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### REFERENCE TO SEQUENCE LISTING, TABLE, OR COMPUTER PROGRAM LISTING

The present application is being filed along with a Sequence Listing in electronic format. The Sequence Listing g is provided as a file entitled CALTE.157C1_ST.26.XML created on Jul. 21, 2025 and is 151,552 bytes in size. The information in the electronic format of the Sequence Listing is incorporated herein by reference in its entirety.

### BACKGROUND

Programmable guide RNAs (gRNAs) play a central role in the CRISPR revolution sweeping biology and medicine by directing the function of Cas protein effectors to a target gene of choice, providing a versatile programmable platform for engineering diverse modes of synthetic regulation in organisms ranging from bacteria to humans. Wildtype Cas9 and Cas12a allow genome editing¹⁻⁴ while mutated catalytically dead Cas9 (dCas9) and nickase variants allow gene editing, silencing, induction,⁵⁻⁸ binding, epigenome editing,⁹ chromatin interaction mapping¹⁰ and regulation,¹¹ and imaging.¹² Hence, gRNA-mediated CRISPR/Cas combines the rich functional vocabulary of different Cas effectors (edit, silence, induce, bind, etc) and the programmability of the gRNA. To target a new gene of choice, all that is needed is to change the sequence of the gRNA.

However, it can be challenging to confine gRNA activity to a desired location and time within an organism. Strategies for achieving temporal control include modulation of gRNA activity using antisense RNAs¹³ and small-molecule induction of gRNAs^{14,15} or Cas9.¹⁶ Spatiotemporal control can be achieved in photoaccessible tissues using light to uncage gRNAs,^{17,18} cleave antisense DNAs,¹⁹ or regulate Cas9.²⁰ Alternatively, Cas9 can be regulated using tissue-specific promoters^{21,22} or microRNAs.²³ Cas9 tolerates, to varying degrees, a variety of modifications to the standard gRNA structure,²⁴⁻²⁶ allowing for introduction of auxiliary domains to provide hooks for regulation by small-molecules,²⁷⁻²⁹ protein-bound RNAs,³⁰ nucleases,³¹ or nuclease-recruiting DNAs or miRNAs.^{31,32}

### SUMMARY OF THE INVENTION

In accordance with some implementations, there is an allosteric conditional guide RNA (cgRNA) comprising a target-binding region and a trigger-binding region, wherein the target-binding region is non-overlapping with the trigger-binding region, wherein the cgRNA is active in the absence of a cognate RNA trigger, wherein the cgRNA is configured to mediate the function of a Cas protein effector on a target gene that binds the target-binding region, and wherein upon hybridization to the cognate RNA trigger, the cgRNA is inactivated, inhibiting further mediation of Cas function on the target gene. In accordance with certain implementations, the allosteric cgRNA may further comprise a Cas handle wherein the target-binding region is 5′ of the Cas handle and the trigger-binding region is 3′ of the Cas handle. In accordance with certain implementations, the allosteric cgRNA may further comprise a first terminator hairpin with an extended loop comprising 5 or more nucleotides wherein the trigger-binding region comprises zero, one, or more nucleotides of a linker 5′-adjacent to the first terminator hairpin, zero, one, or more nucleotides in a 5′ portion of a stem of the first terminator hairpin, and one or more nucleotides in the extended loop of the first terminator hairpin, wherein the cgRNA is inactivated upon hybridization of the cognate RNA trigger to the cgRNA. In accordance with some implementations, the allosteric cgRNA may further comprise a Cas handle with an extended loop wherein the target-binding region is 5′ of the Cas handle and the trigger-binding region comprises a portion of the extended loop of the Cas handle and no nucleotides 5′ of the Cas handle. In accordance with some implementations, the allosteric cgRNA may further comprise a first terminator hairpin with an extended loop comprising 5 or more nucleotides such that the trigger-binding region comprises one or more nucleotides in the extended loop of the Cas handle, and one or more nucleotides in the extended loop of the first terminator hairpin, wherein upon hybridization of the cognate RNA trigger to the cgRNA, the cgRNA is inactivated. In accordance with some implementations, the allosteric cgRNA may further comprise a first terminator hairpin with an extended loop comprising 5 or more nucleotides wherein the trigger-binding region further comprises zero, one, or more nucleotides of a linker 5′-adjacent to the first terminator hairpin, zero, one, or more nucleotides in a 5′ portion of a stem of the first terminator hairpin, and one or more nucleotides in the extended loop of the first terminator hairpin, wherein the cgRNA is inactivated upon hybridization of the cognate RNA trigger to the cgRNA.

In accordance with some implementations, there is an allosteric conditional guide RNA (cgRNA) and an RNA inhibitor strand, wherein the cgRNA comprises a target-binding region and an inhibitor-binding region, and the RNA inhibitor strand comprises a trigger-binding region, wherein the cgRNA is configured to bind to a portion of the trigger-binding region to form a cgRNA:inhibitor complex: wherein the target-binding region is not base-paired to the trigger-binding region in the cgRNA:inhibitor complex; wherein the cgRNA:inhibitor complex is inactive in the absence of a cognate RNA trigger; and wherein upon hybridization of a cognate RNA trigger to the inhibitor, the cgRNA is activated, mediating the function of a Cas protein effector on a target gene that binds the target-binding region. In accordance with some implementations, the cgRNA further comprises a Cas handle wherein the target-binding region is 5′ of the Cas handle and the inhibitor-binding region is 3′ of the Cas handle. In accordance with some implementations, the inhibitor further comprises a toehold of one or more unpaired nucleotides at one or both ends, the cgRNA further comprising a first terminator hairpin with an extended loop comprising 5 or more nucleotides, the cgRNA further comprising an inhibitor-binding region comprising: zero, one, or more nucleotides of a linker 5′-adjacent to the first terminator hairpin; zero, one, or more nucleotides in a 5′ portion of a stem of the first terminator hairpin; and one or more nucleotides in the extended loop of the first terminator hairpin; wherein the cgRNA is inactive in the cgRNA:inhibitor complex, and wherein hybridization of the cognate RNA trigger to the inhibitor displaces the cgRNA from the inhibitor, thereby activating the cgRNA. In accordance with some implementations, the cgRNA further comprises a Cas handle with an extended loop wherein the target-binding region is 5′ of the Cas handle, and wherein the inhibitor-binding region comprises a portion of the extended loop of the Cas handle and no nucleotides 5′ of the Cas handle. In accordance with some implementations, the inhibitor further comprises a toehold of one or more unpaired nucleotides at one or both ends, the cgRNA further comprising a first terminator hairpin with an extended loop comprising 5 or more nucleotides, and the inhibitor-binding region comprising: one or more nucleotides in the extended loop of the Cas handle; and one or more nucleotides in the extended loop of the first terminator hairpin; wherein the cgRNA is inactive in the cgRNA:inhibitor complex, and wherein hybridization of the cognate RNA trigger to the inhibitor displaces the cgRNA from the inhibitor, thereby activating the cgRNA. In accordance with some implementations, the inhibitor comprises a toehold of one or more unpaired nucleotides at one or both ends, the cgRNA further comprising a first terminator hairpin with an extended loop comprising 5 or more nucleotides, and the inhibitor-binding region comprising: one or more nucleotides in the extended loop of the Cas handle; zero, one, or more nucleotides of a linker 5′-adjacent to the first terminator hairpin; zero, one, or more nucleotides of a 5′ portion of a stem of the first terminator hairpin; and one or more nucleotides in the extended loop of the first terminator hairpin; wherein the cgRNA is inactive in the cgRNA:inhibitor complex, and wherein hybridization of the cognate trigger to the inhibitor displaces the cgRNA from the inhibitor, thereby activating the cgRNA.

In accordance with some implementations, there is an allosteric conditional guide RNA (cgRNA) comprising a 5′ fragment (cg5) and a 3′ fragment (cg3), cg5 comprising a target-binding region and a trigger-binding region, and cg3 comprising a cognate RNA trigger: wherein the target-binding region is non-overlapping with the trigger-binding region; wherein cg5 and cg3 are inactive when not bound to each other; and wherein upon hybridization of cg3 to cg5 to form a cg5:cg3 complex, the cgRNA is activated, mediating the function of a Cas protein effector on a target gene that binds the target-binding region. In accordance with some implementations, the fragment cg5 further comprises a Cas handle wherein the target-binding region is 5′ of the Cas handle and the trigger-binding region is 3′ of the Cas handle. In accordance with some implementations, the fragment cg5 further comprises a trigger-binding region comprising a 5′ portion of a stem of a terminator duplex and the fragment cg3 further comprises a 3′ portion of a stem of the terminator duplex, wherein hybridization of cg5 to cg3 forms the terminator duplex, activating the cgRNA.

In accordance with some implementations, there is an allosteric conditional guide RNA (cgRNA) comprising a 5′ fragment (cg5) and a 3′ fragment (cg3), cg5 comprising a target-binding region and a trigger-binding region, and cg3 configured to bind to a portion of the trigger-binding region to form a cg5:cg3 complex: wherein the target-binding region is non-overlapping with the trigger-binding region; wherein the cg5:cg3 complex is active in the absence of a cognate RNA trigger, mediating the function of a Cas protein effector on a target gene that binds the target-binding region; wherein cg5 and cg3 are inactive when not bound to each other; and wherein hybridization of the cognate RNA trigger to cg5 displaces cg3 from cg5, thereby inhibiting further mediation of Cas function on the target gene. In accordance with some implementations, the fragment cg5 further comprises a Cas handle wherein the target-binding region is 5′ of the Cas handle and the trigger-binding region is 3′ of the Cas handle. In accordance with some implementations, the fragment cg5 further comprises a trigger-binding region comprising: a 5′ portion of a stem of a terminator duplex; zero, one, or more nucleotides of a linker 5′-adjacent to the 5′ portion of the stem of the terminator duplex, and a toehold comprising zero, one, or more nucleotides 3′-adjacent to 5′ portion of the stem of the terminator duplex, wherein the fragment cg3 further comprises a 3′ portion of the stem of the terminator duplex, wherein hybridization of cg5 to cg3 forms the terminator duplex within the cg5:cg3 complex, and wherein hybridization of the trigger to cg5 displaces cg3 from cg5, thereby breaking the terminator duplex and inactivating the cgRNA.

In accordance with some implementations, there is an allosteric conditional guide RNA (cgRNA) comprising: a 5′ fragment (cg5) and a 3′ fragment (cg3), cg5 comprising a target-binding region; and cg3 comprising a trigger-binding region, wherein cg5 is configured to bind to a portion of the trigger-binding region to form a cg5:cg3 complex: wherein the target-binding region is not base-paired to the trigger-binding region in the cg5:cg3 complex; wherein the cg5:cg3 complex is active in the absence of a cognate RNA trigger, mediating the function of a Cas protein effector on a target gene that binds the target-binding region; wherein cg5 and cg3 are inactive when not bound to each other; and wherein hybridization of the cognate RNA trigger to cg3 displaces cg5 from cg3, thereby inhibiting further mediation of Cas function on the target gene. In accordance with some implementations, the fragment cg5 further comprises a Cas handle wherein the target-binding region is 5′ of the Cas handle, and wherein the fragment cg3 binds to cg5 3′ of the Cas handle. In accordance with some implementations, the fragment cg3 further comprises a trigger-binding region comprising a 3′ portion of a stem of a terminator duplex, and a toehold comprising zero, one, or more nucleotides 5′-adjacent to the 3′ portion of the stem of the terminator duplex; the fragment cg5 further comprises a 5′ portion of the stem of the terminator duplex; and wherein hybridization of cg5 to cg3 forms the terminator duplex within the cg5:cg3 complex, and wherein hybridization of the cognate RNA trigger to cg3 displaces cg5 from cg3, thereby breaking the terminator duplex and inactivating the cgRNA.

In accordance with some implementations, there is an allosteric conditional guide RNA (cgRNA) comprising a 5′ fragment (cg5) and a 3′ fragment (cg3): wherein cg5 comprises a Cas handle, a target-binding region 5′ of the Cas handle, and a cg3-binding region 3′ of the Cas handle, wherein cg3 comprises a cg5-binding region, and wherein either cg5 or cg3 comprises a trigger-binding region: wherein the target-binding region is non-overlapping with the trigger-binding region and is configured not to bind to the trigger-binding region; wherein cg5 and cg3 are inactive when not bound to each other, wherein in the absence of a cognate RNA trigger, cg5 and cg3 are configured to be inhibited from binding to each other, and wherein upon hybridization of the cognate RNA trigger to either cg5 or cg3, cg5 and cg3 hybridize to form a trigger:cg5:cg3 complex that activates the cgRNA, thereby mediating the function of a Cas protein effector on a target gene that binds the target-binding region. In accordance with some implementations, the fragment cg5 further comprises: a trigger-binding region comprising a first inhibitor region, and a second inhibitor region, wherein in the absence of the cognate RNA trigger, the first inhibitor region is configured to bind to the second inhibitor region, thereby inhibiting binding between cg5 and cg3. In accordance with some implementations, the cognate RNA trigger comprises a cg5-binding region; the trigger-binding region further comprises a toehold of one or more unpaired nucleotides at one or both ends; the cg3-binding region of cg5 comprises a 5′ portion of a stem of a terminator duplex; and the cg5-binding region of cg3 comprises a 3′ portion of the stem of the terminator duplex; wherein upon hybridization of the cognate RNA trigger to cg5, cg5 hybridizes to cg3 to form the terminator duplex, thereby activating the cgRNA. In accordance with some implementations, cg3 further comprises: a trigger-binding region comprising a first inhibitor region, and a second inhibitor region, wherein in the absence of the cognate RNA trigger, the first inhibitor region is configured to bind the second inhibitor region, thereby inhibiting binding between cg5 and cg3. In accordance with some implementations, the cognate RNA trigger comprises a cg3-binding region; the trigger-binding region further comprises a toehold of one or more unpaired nucleotides at one or both ends; the cg5-binding region of cg3 comprises a 3′ portion of a stem of a terminator duplex; and the cg3-binding region of cg5 comprises a 5′ portion of the stem of the terminator duplex, wherein upon hybridization of the cognate RNA trigger to cg3, cg3 hybridizes to cg5 to form the terminator duplex, thereby activating the cgRNA. In accordance with some implementations, cg5 further comprises: a trigger-binding region comprising a first inhibitor region and a second inhibitor region; and wherein cg3 further comprises a third inhibitor region and a fourth inhibitor region, wherein in the absence of a cognate RNA trigger, the first inhibitor region is configured to bind the second inhibitor region and the third inhibitor region is configured to bind to the fourth inhibitor region, thereby inhibiting binding between cg5 and cg3. In accordance with some implementations, the cognate RNA trigger comprises a cg5-binding region; the trigger-binding region of cg5 further comprises a toehold of one or more unpaired nucleotides at one or both ends; the cg3-binding region of cg5 comprises a 5′ portion of a stem of a terminator duplex; and the cg5-binding region of cg3 comprises a 3′ portion of the stem of the terminator duplex comprising a toehold of one or more unpaired nucleotides at one or both ends, wherein upon hybridization of the cognate RNA trigger to cg5, cg5 hybridizes to cg3 to form the terminator duplex, thereby activating the cgRNA. In accordance with some implementations, the trigger-binding region of cg5 is 5′ of the target-binding region. In accordance with some implementations, cg3 comprises a trigger mimic region having a sequence identical to that of the cg5-binding region of the cognate RNA trigger, wherein upon activation of the cgRNA by the cognate RNA trigger, the trigger mimic region is exposed and capable of serving as the cognate RNA trigger for a new copy of the cgRNA comprising a new copy of the 5′ fragment cg5 and a new copy of the 3′ fragment cg3. In accordance with some implementations, the trigger-binding region of cg5 is 3′ of the Cas handle. In accordance with some implementations, the cgRNA additionally comprises a splint as a third fragment wherein the splint comprises a cg3-binding region comprising a fifth inhibitor region and further comprising a toehold of one or more unpaired nucleotides at one or both ends; and a cg5-binding region comprising a sixth inhibitor region; wherein in the absence of a cognate RNA trigger, the fifth inhibitor region is configured to bind the sixth inhibitor region, inhibiting binding of the splint to cg5 and cg5, and wherein upon activation of the cgRNA by the cognate RNA trigger, cg3 hybridizes to the cg3-binding region of the splint and the cg5-binding region of the splint hybridizes to cg5, displacing the trigger from cg5, and catalytically regenerating the trigger which then serves as the cognate RNA trigger for a new copy of the cgRNA comprising a new copy of the 5′ fragment cg5 and a new copy of the 3′ fragment cg3. In accordance with some implementations, the catalytically regenerated trigger serves as the cognate RNA trigger for a new copy of the cgRNA which further comprises a new copy of the splint fragment. In accordance with some implementations, cg3 comprises a trigger mimic region having a sequence identical to that of the cg5-binding region of the cognate RNA trigger, wherein upon activation of the cgRNA by the cognate RNA trigger, the trigger mimic region hybridizes to the trigger-binding region of cg5, displacing the trigger from cg5, and catalytically regenerating the trigger which can then serve as the cognate RNA trigger for a new copy of the cgRNA comprising a new copy of the 5′ fragment cg5 and a new copy of the 3′ fragment cg3.

In accordance with some implementations, there is a method of conditionally inhibiting mediation of Cas function on a target gene, comprising: providing an allosteric conditional guide RNA (cgRNA); and combining the cgRNA with a system comprising a target gene and a Cas protein effector; wherein the cgRNA is active in mediating the function of the Cas protein effector on the target gene in the absence of a cognate RNA trigger, and wherein upon hybridization to the cognate RNA trigger, the cgRNA is inactivated, inhibiting further mediation of Cas function on the target gene.

In accordance with some implementations, there is a method of conditionally mediating the function of a Cas protein effector on a target gene, comprising: providing an allosteric conditional guide RNA (cgRNA) and an RNA inhibitor strand; and combining the cgRNA and RNA inhibitor strand with a system comprising a target gene and a Cas protein effector; wherein in the absence of a cognate RNA trigger, the inhibitor is bound to the cgRNA and the cgRNA is inactive; and wherein upon hybridization of a cognate RNA trigger to the inhibitor, the cgRNA is activated, mediating the function of a Cas protein effector on the target gene.

In accordance with some implementations, there is a method of conditionally mediating the function of a Cas protein effector on a target gene, comprising: providing an allosteric conditional guide RNA (cgRNA) comprising a 5′ fragment (cg5) and a 3′ fragment (cg3); and combining the cgRNA with a system comprising a target gene and a Cas protein effector; wherein cg5 and cg3 are inactive when not bound to each other; and wherein upon hybridization of cg3 to cg5, the cgRNA is activated, mediating the function of a Cas protein effector on the target gene.

In accordance with some implementations, there is a method of conditionally inhibiting mediation of Cas function on a target gene, comprising: providing an allosteric conditional guide RNA (cgRNA) comprising a 5′ fragment (cg5) and a 3′ fragment (cg3); and combining the cgRNA with a system comprising a target gene and a Cas protein effector; wherein in the absence of a cognate RNA trigger cg5 is bound to cg3 and the cgRNA is active; and wherein hybridization of the cognate RNA trigger to cg5 displaces cg3 from cg5, thereby inhibiting further mediation of Cas function on the target gene.

In accordance with some implementations, there is a method of conditionally inhibiting mediation of Cas function on a target gene, comprising: providing an allosteric conditional guide RNA (cgRNA) comprising a 5′ fragment (cg5) and a 3′ fragment (cg3); and combining the cgRNA with a system comprising a target gene and a Cas protein effector; wherein in the absence of a cognate RNA trigger cg5 is bound to cg3 and the cgRNA is active; and wherein hybridization of the cognate RNA trigger to cg3 displaces cg5 from cg3, thereby inhibiting further mediation of Cas function on the target gene.

In accordance with some implementations, there is a method of conditionally mediating the function of a Cas protein effector on a target gene, comprising: providing an allosteric conditional guide RNA (cgRNA) comprising a 5′ fragment (cg5) and a 3′ fragment (cg3); and combining the cgRNA with a system comprising a target gene and a Cas protein effector; wherein in the absence of a cognate RNA trigger, cg5 and cg3 are inhibited from binding to each other and the cgRNA is inactive; and wherein upon hybridization of the cognate RNA trigger to either cg5 or cg3, cg5 and cg3 hybridize to form a trigger:cg5:cg3 complex that activates the cgRNA, thereby mediating the function of a Cas protein effector on the target gene.

In accordance with some implementations of any of the foregoing, one or more of the following may also be present: the trigger is an RNA; the trigger is or is a subsequence of an mRNA, an rRNA, a lncRNA, a miRNA, or a tRNA; the cgRNA is expressed in a cell; the cgRNA is chemically synthesized; the cgRNA, cgRNA fragment, RNA inhibitor strand, and/or trigger further comprises one or more additional regions at the 5′ and/or the 3′ end; the cgRNA, cgRNA fragment, RNA inhibitor strand, and/or trigger further comprises one or more chemical modifications that alter one or more of degradation properties, affinity, biological activity, and/or delivery properties of the cgRNA; the cgRNA, cgRNA fragment, RNA inhibitor strand, and/or trigger comprises one or more chemical modifications selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′0-Methyl (20-Me) modification, and 2′0-(2-methoxyethyl) (20-MOE) modification; and the cgRNA works in conjunction with Cas to mediate cell-selective induction, silencing, editing, or binding of a target gene.

In accordance with some implementations of any of the foregoing, the allosteric cgRNA wherein an RNA trigger, RNA helper, and/or RNA inhibitor further comprises a protective element (PEL), wherein none, some, or all of the PEL sequence is derived from a component of a viral xrRNA sequence, and wherein the PEL reduces degradation of the RNA trigger, RNA helper, and/or RNA inhibitor in a prokaryotic or eukaryotic cell. In accordance with some implementations of any of the foregoing, the allosteric cgRNA further wherein the cgRNA and/or one or more cgRNA fragments further comprise a protective element (PEL), wherein none, some, or all of the PEL sequence is derived from a component of a viral xrRNA, and wherein the PEL reduces degradation of the cgRNA in a prokaryotic or eukaryotic cell.

### BRIEF DESCRIPTION OF THE DRAWINGS

FIGS. 1A-1B depict the logic, function, structure, and interactions of a standard guide RNA (gRNA).

FIGS. 2A-2B depict the logic and function of a conditional guide RNA (cgRNA).

FIGS. 3A-3E depict applications of cell-selection spatiotemporal control of CRISPR/Cas regulation.

FIGS. 4A-4C depict the logic, function, and mechanism of allosteric ON→OFF terminator switch cgRNAs (Mechanism 1A).

FIGS. 5A-5C depict the logic, function, and mechanism of allosteric OFF→ON terminator switch cgRNAs (Mechanism 1B).

FIGS. 6A-6C depict the logic, function, and mechanism of allosteric ON→OFF splinted switch cgRNAs (Mechanism 2A).

FIGS. 7A-7C depict the logic, function, and mechanism of allosteric OFF→ON splinted switch cgRNAs (Mechanism 2B).

FIGS. 8A-8C depict the logic, function, and mechanism of allosteric OFF→ON split-terminator switch cgRNAs (Mechanism 4A).

FIGS. 9A-9E depict the logic, function, and mechanism of allosteric ON→OFF split-terminator switch cgRNAs (Mechanisms 4B and 4C).

FIGS. 10A-10C depict the interactions between allosteric cgRNAs, triggers, and Cas.

FIGS. 11A-11F demonstrate ON→OFF conditional logic in bacteria using allosteric terminator switch cgRNAs.

FIG. 12A-12F demonstrate ON→OFF conditional logic in bacteria using allosteric splinted switch cgRNAs.

FIG. 13A-13J demonstrate ON→OFF conditional logic in human cells using allosteric terminator switch cgRNAs.

FIG. 14A-14H demonstrate OFF→ON conditional logic in human cells using allosteric split-terminator switch cgRNAs with a 4-bp terminator duplex.

FIG. 15A-15H demonstrate OFF→ON conditional logic in human cells using allosteric split-terminator switch cgRNAs with a 10-bp terminator duplex.

FIG. 16A-16E demonstrate OFF→ON conditional logic in human cells using allosteric split-terminator switch cgRNAs with terminator duplexes of different lengths (40, 30, 20, 10, 8, 6, 4 bp).

FIG. 17A-17C demonstrate that 3′ truncation of a standard gRNA can lead to complete inactivation, providing the basis for engineering cgRNAs with a clean OFF state.

FIGS. 18A-18F demonstrate ON→OFF conditional logic in bacteria using allosteric terminator switch cgRNAs and mRNA triggers.

FIGS. 19A-19B depict the mechanism and target test tubes for computational sequence design of a library of orthogonal ON→OFF terminator switch cgRNAs/triggers.

FIGS. 20A-20B depict the mechanism and target test tubes for computational sequence design of a library of orthogonal OFF→ON split-terminator switch cgRNAs/triggers.

FIGS. 21A-21E demonstrate an allosteric ON→OFF cgRNA functioning in a multicellular organism

FIGS. 22A-22E demonstrate an allosteric OFF→ON cgRNA functioning in a multicellular organism

FIGS. 23A-23C depict the logic, function, and mechanism of allosteric ON→OFF tandem switch cgRNAs (Mechanism 3A).

FIGS. 24A-24C depict the logic, function, and mechanism of allosteric OFF→ON tandem switch cgRNAs (Mechanism 3B).

FIGS. 25A-25C depict the logic, function, and mechanism of allosteric OFF→ON 5′-inhibited split-terminator switch cgRNAs (Mechanism 5).

FIGS. 26A-26C depict the logic, function, and mechanism of allosteric OFF→ON 3′-inhibited split-terminator switch cgRNAs (Mechanism 6).

FIGS. 27A-27C depict the logic, function, and mechanism of allosteric OFF→ON 5′- and 3′-inhibited split-terminator switch cgRNAs (Mechanism 7).

FIGS. 28A-28D depict the logic, function, and mechanism of allosteric OFF→ON 5′- and 3′-inhibited split-terminator switch cgRNAs (Mechanism 8).

FIGS. 29A-29D depict the logic, function, and mechanism of allosteric OFF→ON 5′- and 3′-inhibited split-terminator switch cgRNAs (Mechanism 9).

FIGS. 30A-30D demonstrate allosteric OFF→ON split-terminator switch cgRNAs functioning in bacteria.

FIGS. 31A-31E demonstrate allosteric ON→OFF split-terminator switch cgRNAs functioning in human cells.

FIGS. 32A-32F demonstrate allosteric ON→OFF split-terminator switch cgRNAs functioning in bacteria.

FIGS. 33A-33D demonstrate allosteric OFF→ON 5′- and 3′-inhibited split-terminator switch cgRNAs functioning in bacteria.

### DETAILED DESCRIPTION OF THE PREFERRED EMBODIMENT

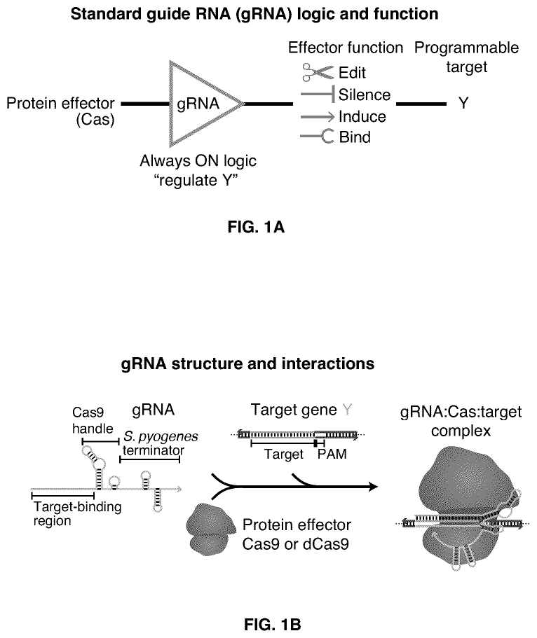

Programmable guide RNAs (gRNAs) play a central role in the CRISPR revolution sweeping biology and medicine by directing the function of Cas protein effectors to a target gene of choice (FIG. 1A), providing a versatile programmable platform for engineering diverse modes of synthetic regulation in organisms ranging from bacteria to humans. Wildtype Cas9 and Cas12a allow for genome editing¹⁻⁴ while mutated catalytically dead Cas9 (dCas9) and nickase variants allow for gene editing, silencing, induction,⁵⁻⁸ binding, epigenome editing,⁹ chromatin interaction mapping¹⁰ and regulation,¹¹ and imaging.¹² Hence, gRNA-mediated CRISPR/Cas combines the rich functional vocabulary of different Cas effectors (edit, silence, induce, bind, etc) and the programmability of the gRNA. To target a new gene of choice, all that is needed is to change the sequence of the gRNA.

However, the fact that gRNAs are constitutively active is a significant limitation, making it challenging to confine gRNA activity to a desired location and time within an organism. Strategies for achieving temporal control include modulation of gRNA activity using antisense RNAs¹³ and small-molecule induction of gRNAs^{14,15} or Cas9.¹⁶ Spatiotemporal control can be achieved in photoaccessible tissues using light to uncage gRNAs, 17,18 cleave antisense DNAs,¹⁹ or regulate Cas9.²⁰ Alternatively, Cas9 can be regulated using tissue-specific promoters^{21,22} or microRNAs.²³ Cas9 tolerates, to varying degrees, a variety of modifications to the standard gRNA structure (FIG. 1B),²⁴⁻²⁶ allowing for introduction of auxiliary domains to provide hooks for regulation by small-molecules,²⁷⁻²⁹ protein-bound RNAs,³⁰ nucleases,³¹ or nuclease-recruiting DNAs or miRNAs.^{31,32} As appreciated herein, for generality, it can be desirable to control gRNA regulatory scope in a manner that is both conditional and programmable, and for simplicity, to leverage dynamic RNA nanotechnology without relying on the functionality of additional pathways.

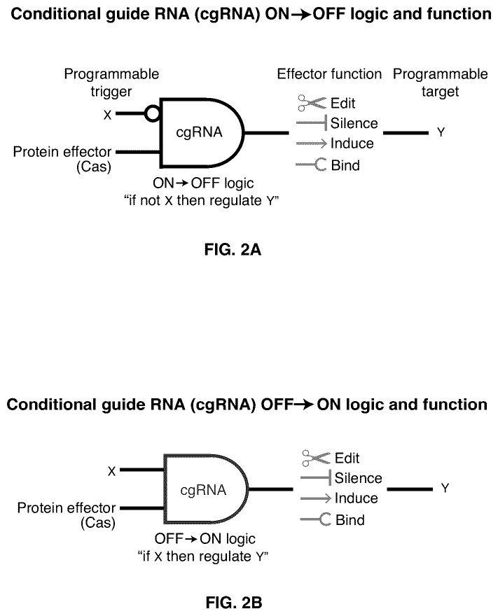

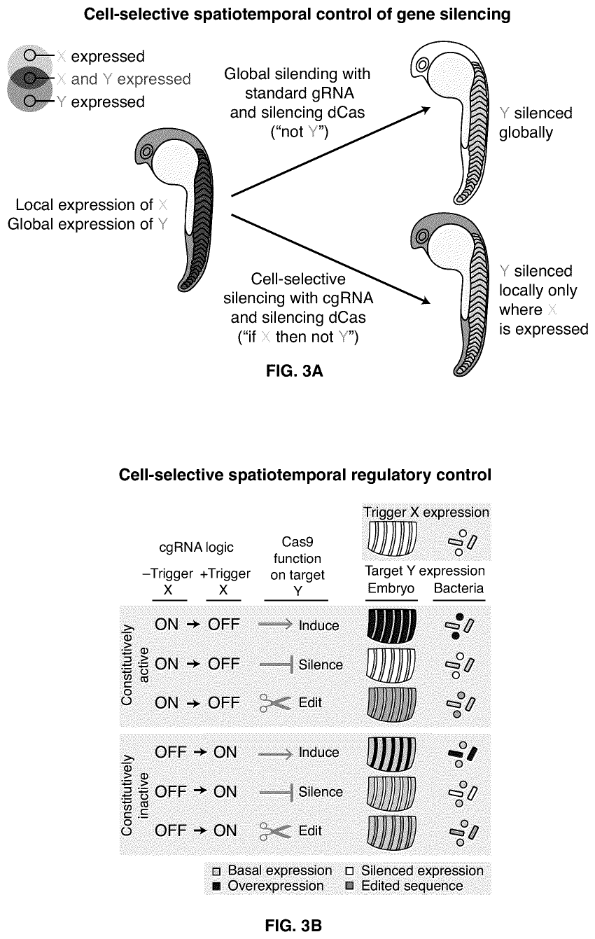

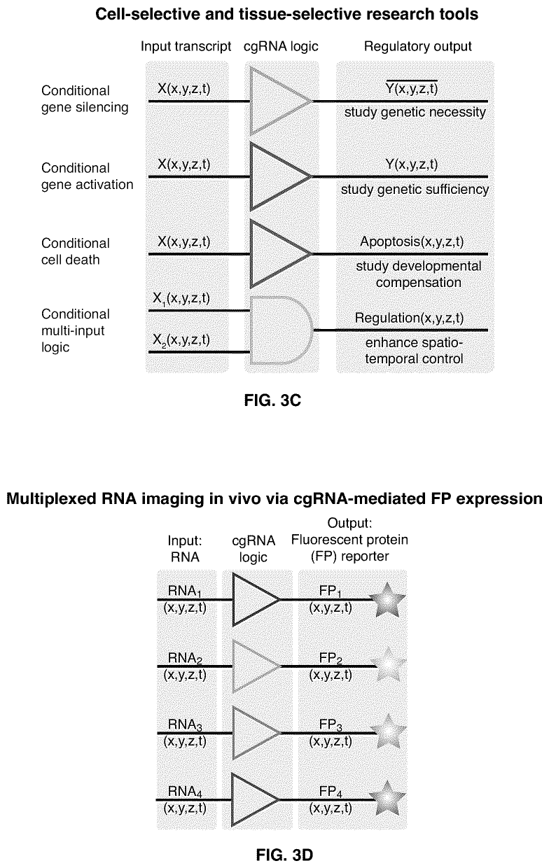

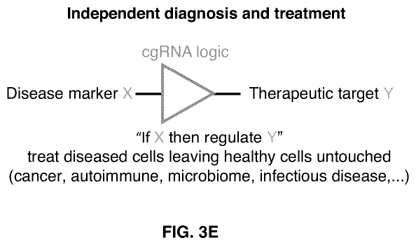

To exert programmable control over the scope of gRNA activity, conditional guide RNAs (cgRNAs) change conformation in response to an RNA trigger X, conditionally directing the function of Cas to a target gene Y (FIG. 2).^{26,33,34} Unlike a standard gRNA, a cgRNA is programmable at two levels, with the trigger-binding sequence controlling the scope of cgRNA activity and the target-binding sequence determining the subject of Cas activity. Hybridizing to the trigger changes the cgRNA conformation to perform sequence transduction between X and Y and shape transduction between active/inactive states. The disclosure herein relates to cgRNAs that are allosteric so that the sequence of the target gene Y places no restriction on the sequence of the RNA trigger X, allowing for independent control over the regulatory scope (using X) and the regulatory target (using Y). In some embodiments, cgRNA mechanisms implement ON→OFF logic (conditional inactivation by trigger X; FIG. 2A). In some embodiments, cgRNA mechanisms implement OFF→ON logic (conditional activation by trigger X; FIG. 2B). In some embodiments, cgRNAs work in concert with Cas variants that either edit, silence, induce, or bind the target Y (FIG. 2), creating opportunities for diverse modes of tissue-selective spatiotemporal control over regulation (see for example FIG. 3). In some embodiments, cgRNAs work in concert with Cas variants that mediate induction, silencing, editing, binding, epigenome editing, chromatin interaction mapping and regulation, or imaging of a target gene.

In some embodiments, cgRNAs open the possibility of restricting synthetic regulation to a desired cell type, tissue, or organ. This can be achieved by selecting an endogenous RNA trigger X with the desired spatial and temporal expression profile, allowing for spatiotemporal control over regulation (FIG. 3A). In some embodiments, cgRNAs open the possibility of restricting synthetic regulation to a desired cell type, tissue, or organ without engineering the organism. FIG. 3B illustrates a variety of modes of cell-selective spatiotemporal regulatory control that can be implemented by combining the conditionality of cgRNA logic (ON→OFF and OFF→ON) and the functionality of Cas variants (edit, silence, induce, bind, etc). In some embodiments, cgRNAs can be used as cell-selective and tissue-selective research tools (FIG. 3C): conditional gene silencing would probe genetic necessity, conditional gene induction would probe genetic sufficiency, conditional cell death would probe developmental compensation. In some embodiments, to shift conditional regulation to a different tissue or developmental stage, the sequence of a cgRNA is simply redesigned to recognize a different input X with the desired spatial and temporal expression profile. In some embodiments, cgRNAs can be used to mediate in vivo imaging of a target RNA using the cgRNA to recognize the RNA of interest and mediate expression of a fluorescent protein reporter. In some embodiments, multiple cgRNAs recognizing different target RNAs and inducing spectrally distinct different fluorescent protein reporters can be used for multiplexed in vivo RNA imaging. In some embodiments, in a model organism with N fluorescent proteins integrated into the genome, a set of N target RNAs can be imaged in vivo using a set of N cgRNAs to induce the fluorescent proteins upon detection of the corresponding target RNAs. In some embodiments, to switch to imaging a new set of N target RNAs, no genome engineering is required as this can be achieved simply by using a new set of N cgRNAs. In some embodiments, cgRNAs also provide a framework for conditional chemotherapies (“if X then regulate Y”) with X as a programmable disease marker and Y as an independent programmable therapeutic pathway, allowing for selective treatment or killing of diseased cells leaving healthy cells untouched (FIG. 3E).

The repurposing of RNA-guided CRISPR effectors through development of modified guide RNAs (gRNAs) and CRISPR-associated (Cas) proteins has yielded a suite of powerful tools for biological research, synthetic biology, and medicine. Precision genome editing has been achieved in a variety of organisms using gRNAs to direct the nuclease activity of Cas9 and Cas12a (Cpf1) to a target gene of choice.¹⁻⁴ Mutation of the nuclease domains to produce a catalytically dead Cas9 (dCas9) has allowed for silencing of genetic expression via inhibition of transcriptional elongation,^{5,6} or induction (or silencing) of genetic expression using dCas9 fusions that incorporate transcriptional regulatory domains.⁷ Other dCas9 fusions have mediated target-binding to allow for visualization of genomic loci,^{35,36} epigenetic modification,³⁷ and single-base editing at a specific genomic locus,^{4,38} chromatin interaction mapping¹⁰ and regulation,¹¹ and imaging.¹² Hence, gRNA:effector complexes combine the benefits of the rich functional vocabulary of the protein effector (edit, silence, induce, bind) and the programmability of the gRNA in targeting effector activity to a gene of choice.

Because gRNAs are constitutively active, additional measures are needed to restrict effector activity to a desired location and time. Temporal control can be achieved by small-molecule induction of gRNAs^{14,15} or Cas9,¹⁶ but this comes with limitations in terms of multiplexing and spatial control. In some settings, spatiotemporal control can be achieved by regulation of Cas9 via photoactivation²⁰ or via tissue-specific promoters^{21,22} or microRNAs,²³ which comes with the unwelcome restriction that all gRNAs are subject to the same regulatory scope. Cas9 activity is tolerant to significant modifications to the standard gRNA structure.²⁴⁻²⁶ The introduction of auxiliary domains can allow for conditional control of gRNA activity via structural changes induced by small-molecules,²⁷⁻²⁹ protein-bound RNAs,³⁰ nucleases,³¹ or nuclease-recruiting DNAs.³¹ Alternatively, the activity of standard gRNAs can be modulated by antisense RNAs¹³ or by photolysis of antisense DNAs incorporating photocleavable groups.¹⁹

For generality, it is useful to control the regulatory scope of a gRNA in a manner that is both conditional and programmable. Conditional guide RNAs (cgRNAs) achieve this goal by changing conformation in response to an RNA trigger X to conditionally direct the function of a Cas effector to a target gene Y.^{26,33,34,39} Unlike a standard gRNA, a cgRNA is programmable at two levels, with the trigger-binding sequence controlling the scope of cgRNA activity and the target-binding sequence determining the subject of effector activity. Functionally, the cgRNA performs sequence transduction between X and Y as well as shape transduction between active/inactive conformations. cgRNA activity can be engineered to toggle either ON→OFF or OFF→ON in response to a cognate RNA trigger X; this conditional control can be exerted over Cas (for example, Cas9 or dCas9) variants that either, for example, edit, silence, induce, or bind the target Y (FIG. 2). For example, by selecting an endogenous transcript X with a desired spatiotemporal expression profile during development, the downstream regulatory effect on target Y could be restricted to a desired tissue and developmental stage within a model organism (FIGS. 3A and 3B). Alternatively, in a therapeutic context, X can be a disease marker and Y an independent therapeutic target, allowing for selective treatment or killing of diseased cells leaving healthy cells untouched (FIG. 3E).

FIG. 1A depicts the logic and function of a standard guide RNA (gRNA). A standard gRNA is always ON, unconditionally directing the activity of a protein effector to a target Y; different Cas9, dCas9, and/or Cas variants implement different functions (for example, edit, silence, induce, bind). FIG. 1B depicts structure and interactions of a standard gRNA. From 5′ to 3′, a standard gRNA comprises: a target-binding region, a Cas handle recognized by the protein effector, and a terminator region.

For some embodiments, FIG. 2 depicts the logic and function of a conditional guide RNA (cgRNA). For some embodiments, a cgRNA changes conformation in response to a programmable trigger X to conditionally direct the activity of a protein effector to a programmable target Y. For some embodiments, FIG. 2A depicts ON→OFF logic with a constitutively active cgRNA that is conditionally inactivated by X. For some embodiments, FIG. 2B depicts OFF→ON logic with a constitutively inactive cgRNA that is conditionally activated by X.

For some embodiments, FIG. 3 illustrates applications of cell-selective regulation of CRISPR/Cas function using cgRNAs. FIG. 3A contrasts global silencing (top arrow) of target gene Y using silencing dCas9 and a standard gRNA that implements the unconditional logic “silence Y” to cell-selective silencing (bottom arrow) of target gene Y using silencing dCas9 and a conditional cgRNA, such that Y is silenced locally only where X is expressed. For some embodiments, FIG. 3B illustrates diverse modes of cell-selective spatiotemporal regulatory control using cgRNA conditional logic (ON→OFF or OFF→ON) and different Cas9 functional variants (induce, silence, edit, bind, etc). ON→OFF and OFF→ON cgRNAs produce inverted regulatory patterns on target Y in response to a given pattern for trigger X. For some embodiments, FIG. 3C illustrates cell-selective and tissue-selective tools. For example, conditional gene silencing (“if gene X is transcribed, silence independent gene Y”) can be used to probe genetic necessity, conditional gene activation (“if gene X is transcribed, activate independent gene Y”) can be used to probe genetic sufficiency, and conditional cell death (“if gene X is transcribed, induce apoptosis”) can be used to probe developmental compensation. In each case, conditional regulation is mediated by a cgRNA whose activity is toggled by a programmable trigger X. For some embodiments, by selecting a trigger X with the desired spatial and temporal expression profiles, the regulatory function is restricted to a desired cell type, tissue, or organ within an organism, mixture of cells, or ecosystem. For some embodiments, to shift conditional regulation to a different tissue type or time point, the cgRNAs can be programmed to recognize a different trigger X. For some embodiments, to enhance cell-selective spatiotemporal control in multi-cellular settings (e.g., within embryos or bacterial mixtures), multi-input conditional logic (operating on two or more inputs X₁, X₂, . . . ) using AND gates can be used to narrow the scope of regulation on Y; alternatively, OR gates can be used to broaden the scope of regulation on Y. In some embodiments, AND logic is implemented using split-cgRNAs that are functional only in the presence of both X₁ and X₂. In some embodiments, OR logic is executed using multiple cgRNA variants that accept different inputs (X₁, X₂, . . . ) but target the same output Y. FIG. 3D illustrates cgRNA-mediated cell-selective reporter regulation for multiplexed in vivo RNA imaging. In some embodiments, 4 cgRNAs each detect a different mRNA input (mRNA₁, mRNA₂, mRNA₃, mRNA₄) that serves as an RNA trigger, activating the corresponding cgRNA to induce the corresponding spectrally distinct FP reporter (FP₁, FP₂, FP₃, FP₄). In some embodiments, after once optimizing a plasmid-based reporter system expressing inducing dCas9, the 4 FP reporters, and the 4 cgRNAs, imaging a new set of mRNAs requires only updating the sequences of the cgRNAs to accept new mRNAs as triggers. In some embodiments, this cgRNA approach offers important conceptual advantages relative to FP fusion methods, which have revolutionized the study of genetic expression,⁴³⁻⁴⁵ but have the well-known drawbacks that a new fusion must be engineered for each gene of interest, that it is difficult to determine whether fusions affect the expression or function of target proteins, and that fusion methods are not applicable to imaging non-protein gene products such as coding and non-coding RNAs. In some embodiments, cgRNAs eliminate these issues by replacing the conventional physical link of FP fusion approaches with a logical link executed by cgRNAs that execute conditional gene induction, allowing for spatiotemporal monitoring of gene expression levels in living chick embryos without the need to modify the imaged molecules (mRNA₁, mRNA₂, mRNA₃, mRNA₄) in any way. FIG. 3E depicts the conditional logic using cgRNAs as conditional chemotherapies: “if disease marker X then regulate therapeutic target Y”. In some embodiments, X is a programmable disease marker and Y is an independent therapeutic target, allowing for selective treatment or killing of diseased cells (the subset of cells containing X) while leaving healthy cells untouched (the subset of cells lacking X). In some embodiments, cgRNAs allow for independent diagnosis (detection of disease marker X) and treatment (regulation or editing of independent therapeutic target Y).

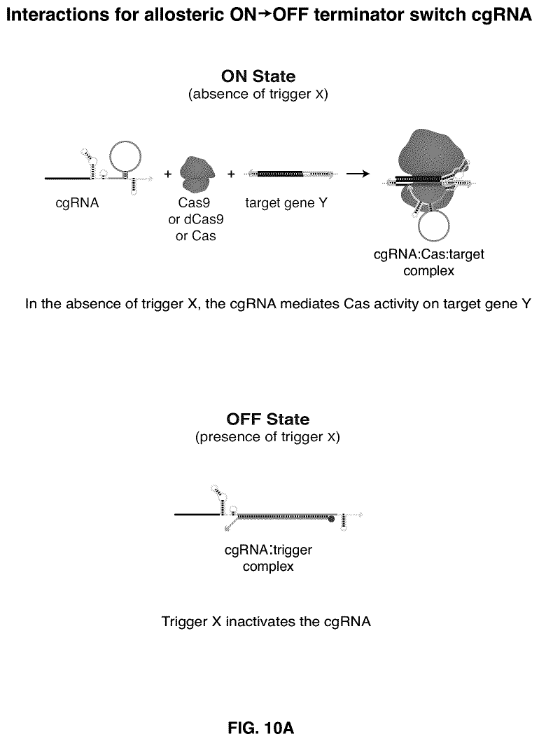

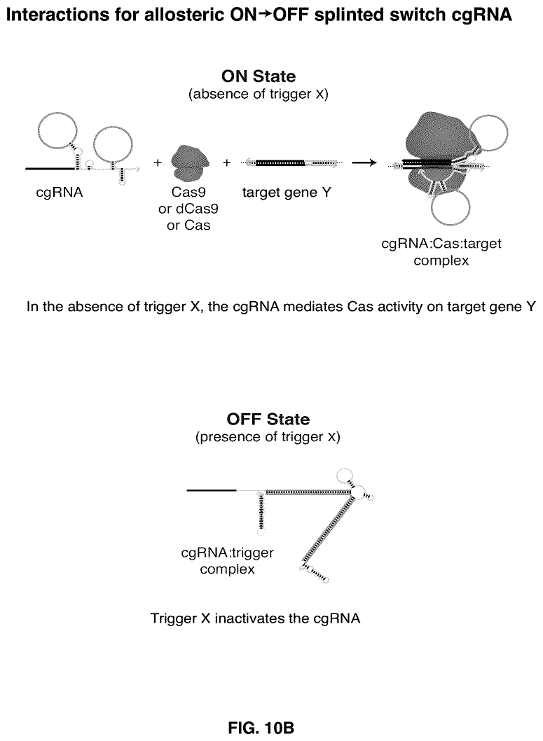

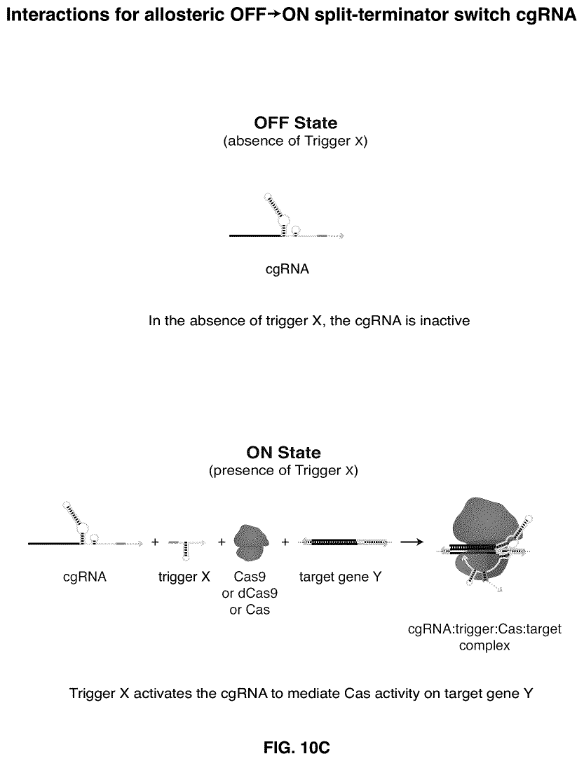

For some embodiments, FIG. 10 depicts interactions between allosteric cgRNAs, RNA triggers, and Cas9, dCas9 or Cas. For some embodiments, FIG. 10A depicts interactions for an allosteric ON→OFF terminator switch cgRNA. In the ON state, the terminator switch cgRNA is constitutively active, directing the function of protein effector Cas9, dCas9, or Cas to a target gene Y in the absence of trigger. The extended loop and modified sequence domains in the terminator region are intended not to interfere with the activity of the cgRNA:Cas complex. In the OFF state, in the presence of RNA trigger X, hybridization of the trigger forms a structure incompatible with cgRNA mediation of Cas9, dCas9, and/or Cas function. For some embodiments, FIG. 10B depicts interactions for an allosteric ON→OFF splinted switch cgRNA. In the ON state, the splinted switch cgRNA is constitutively active, directing the function of protein effector Cas9, dCas9, or Cas to a target gene Y in the absence of trigger. The extended loops in the Cas9 handle and terminator region are intended not to interfere with the activity of the cgRNA:Cas complex. In the OFF state, in the presence of RNA trigger X, hybridization of the trigger forms a splint that is structurally incompatible with cgRNA mediation of Cas9, dCas9, and/or Cas function. For some embodiments, FIG. 10C depicts interactions for an allosteric OFF→ON split-terminator switch cgRNA. In the OFF state, the split-terminator switch cgRNA is constitutively inactive. In the absence of RNA trigger X, the cgRNA is incapable of directing the function of the protein effector Cas9, dCas9, and/or Cas. In the ON state, the complex of cgRNA and trigger X mediates the function of the protein effector Cas9, dCas9, or Cas on the target gene Y. The modified sequence domains in the terminator duplex do not to interfere with the activity of the cgRNA:trigger:Cas complex.

### Definitions

“Nucleic acids” as used herein includes oligomers of RNA, DNA, 2′OMe-RNA, LNA, PNA, XNA, chemically modifications thereof, synthetic analogs of RNA or DNA, any other material capable of base-pairing, one or more chemical linkers not capable of base-pairing, or any combination thereof. Nucleic acids may include analogs of DNA or RNA having modifications to either the bases or the backbone. For example, nucleic acid, as used herein, includes the use of peptide nucleic acids (PNA). The term “nucleic acids” also includes chimeric molecules. The phrase includes artificial constructs as well as derivatives etc. The phrase includes, for example, any one or more of DNA, RNA, 2′OMe-RNA, LNA, XNA, synthetic nucleic acid analogs, and PNA. The phrase also includes oligomers of RNA, DNA, 2′OMe-RNA, LNA, PNA, XNA and/or other nucleic acid analogs with or without chemical linkers between nucleic acid segments.

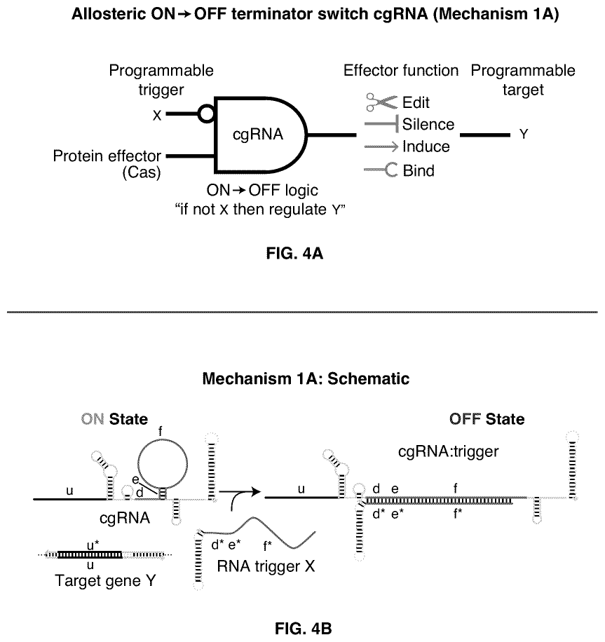

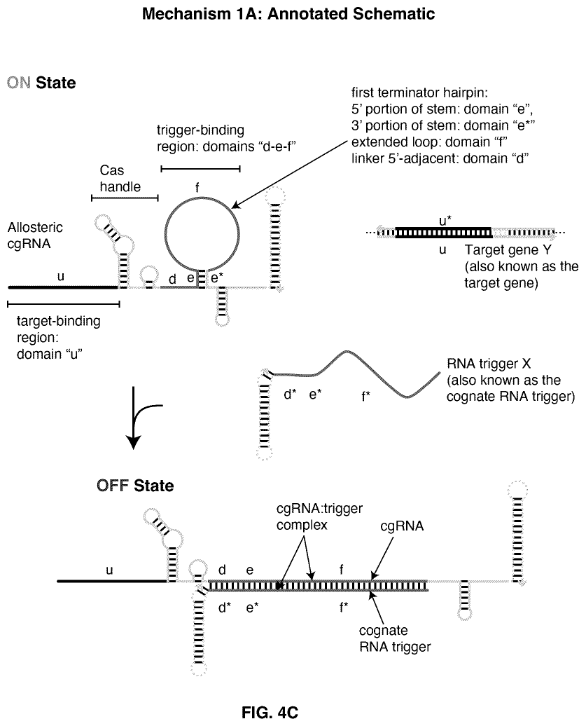

A “nucleic acid strand” refers to an oligomer of nucleotides (typically listed from 5′ to 3′). In diagrams, a nucleic acid strand is depicted with an arrowhead at the 3′ end. A nucleic acid strand may comprise one or more “regions” and/or “sequence domains” (equivalently “domains). For example, FIG. 4C depicts a nucleic acid strand (labeled “Allosteric cgRNA”) containing a “target-binding region” comprising domain “u”, a “Cas handle” region, a “trigger binding region” comprising domains “d”, “e”, and “f”, and other regions and domains. A “secondary structure” of a nucleic acid strand is defined by a set of base pairs (for example, Watson-Crick base pairs [A-U or C-G] or wobble base pairs [G-U] for RNA).

Two “complementary” sequence domains can base-pair to each other (i.e., hybridize) to form a “duplex” or “stem”, representing one or more consecutive base pairs between two regions (or equivalently, one or more consecutive base pairs between two sequence domains). For example, in FIG. 4C, domain “e*” is complementary to sequence domain “e”, allowing for hybridization to form a “duplex” or “stem”. In some settings it is convenient to designate complementary sequence domains using matching domain names with and without an asterisk (for example, domain “e*” complementary to domain “e”). Complementarity may also be specified independent of the sequence domain names. For example, domain “b” may be specified as complementary to domain “c”. The complementarity between two complementary sequence domains may be partial, such that when they base-pair to each other to form a duplex (or stem), the base pairs within the duplex (or stem) may have one or more mismatches interspersed between them (i.e., one or more unpaired bases interspersed between the base pairs within the duplex). In some embodiments, a duplex (or stem) comprises, consists, or consists essentially of 1, 2, 3, 4, 5, 6, 7, 8, 9, 10, 11, 12, 13, 14, 15, 16, 17, 18, 19, 20, or more consecutive base pairs between two segments. In some embodiments a duplex (or stem) comprises, consists, or consists essentially of 5, 10, 15, 20, 25, 30, 35, 40, 45, or 50 consecutive base pairs (or any integer number of consecutive base pairs in between any of these values) between two segments. In some embodiments a duplex (or stem) comprises, consists, or consists essentially of 10, 20, 30, 40, 50, 60, 70, 80, 90, or 100 consecutive base pairs (or any integer number of consecutive base pairs in between any of these values) between two segments). In some embodiments a duplex (or stem) comprises, consists, or consists essentially of 100, 200, 300, 400, or 500 consecutive base pairs (or any integer number of consecutive base pairs in between any of these values) between two segments. In some embodiments, a duplex (or stem) comprises, consists, or consists essentially of 2, 3, 4, 5, 6, 7, 8, 9, 10, 11, 12, 13, 14, 15, 16, 17, 18, 19, 20, or more base pairs between two segments wherein 1, 2, 3, 4, 5, 6, 7, 8, 9, 10, 11, 12, 13, 14, 15, 16, 17, 18, 19, 20, or more unpaired bases are interspersed at one or more locations between the base pairs. In some embodiments a duplex (or stem) comprises, consists, or consists essentially of 5, 10, 15, 20, 25, 30, 35, 40, 45, or 50 base pairs (or any integer number of base pairs in between any of these values) between two segments wherein 1, 5, 10, 15, 20, 25, 30, 35, 40, 45, or 40 unpaired bases (or any integer number of unpaired bases between any of these values) are interspersed at one or more locations between the base pairs. In some embodiments a duplex (or stem) comprises, consists, or consists essentially of 10, 20, 30, 40, 50, 60, 70, 80, 90, or 100 base pairs (or any integer number of base pairs in between any of these values) between two segments wherein 1, 10, 20, 30, 40, 50, 60, 70, 80, 90, or 100 unpaired bases (or any integer number of unpaired bases between any of these values) are interspersed at one or more locations between the base pairs. In some embodiments a duplex (or stem) comprises, consists, or consists essentially of 100, 200, 300, 400, or 500 base pairs (or any integer number of base pairs in between any of these values) between two segments wherein 1, 100, 200, 300, 400, or 500 unpaired bases (or any integer number of unpaired bases between any of these values) are interspersed at one or more locations between the base pairs. In some embodiments, a duplex (or stem) comprising N base pairs between 2 segments further comprises 1, 2, 3, 4, 5, 6, 7, 8, 9, 10 or more mismatches corresponding to bases that are unpaired. In some embodiments, a duplex (or stem) comprising N base pairs between 2 segments further comprises 0% N, 1% N, 2% N, 5% N, 10% N, 20% N, 50% N, 100% N, or 200% N or more mismatches (or any percentage of N mismatches intermediate to the stated values) corresponding to bases that are unpaired.

A “hairpin” is a nucleic acid secondary structure comprising from 5′ to 3′: a 5′ portion of a stem, an unpaired (single-stranded) loop, and a 3′ portion of the stem, wherein the 5′ portion of the stem is base-paired to the 3′ portion of the stem.

Within a nucleic acid secondary structure, a “toehold” is a region or domain comprising one or more unpaired nucleotides, wherein the toehold serves as a nucleation site for binding another nucleic acid strand.

A “Cas handle” is a binding site for a Cas protein effector.

A “conditional guide RNA (cgRNA)” conditionally mediates the function of a Cas protein effector on a target gene depending on the presence/absence of a cognate RNA trigger. In some embodiments, cgRNAs implement ON→OFF logic (conditional inactivation by a cognate RNA trigger; for example FIG. 2A). In some embodiments, cgRNAs implement OFF→ON logic (conditional activation by a cognate RNA trigger; for example FIG. 2B). In some embodiments, cgRNAs work in concert with Cas variants that either edit, silence, induce, or bind the target gene (for example, FIG. 2).

A cgRNA is termed “allosteric” if the cognate RNA trigger toggles the activity of the cgRNA without interacting with the target-binding site within the cgRNA, allowing for the sequence of the cognate RNA trigger to be selected independently of the sequence of the target gene.

As used herein, “combining” encompasses any act or situation where at least two elements are able to interact, including, for example, adding one to the other, allowing the two elements to interact, exposing the two elements to each other, placing or having arranged the elements in a situation where they can interact, etc.

As used herein, the term “providing” encompasses any way to provide the denoted material, including for example, having, obtaining, creating, causing to be created, suppling, etc. the denoted material. This can be done directly (such as the provision of an RNA molecule itself) or indirectly (such as the provision of an DNA molecule that is to be transcribed into the RNA molecule). In some embodiments, this process can be an independent process (such as by obtaining an RNA segment), or it can be part of another process in the method (such as by providing an DNA sequence that is then transcribed into an RNA sequence).

As used in some embodiments herein, the term “mediating” can include one or more of facilitating, directing, or enabling.

In some embodiments, an “inactive” cgRNA is said to be “activated” by a cognate RNA trigger if the trigger increases the cgRNA-mediated function of a Cas protein effector on a target gene by 20%, 50%, 90%, 100%, 200%, 500%, 1000%, or more, or any percentage intermediate to the stated values. In some embodiments, an “inactive” cgRNA is said to be “activated” by a cognate RNA trigger if the trigger increases the cgRNA-mediated function of a Cas protein effector on a target gene by 1.2-fold, 1.5-fold, 2-fold, 5-fold, 10-fold, 20-fold, 50-fold, 100-fold, 200-fold, 500-fold, 1000-fold, 2000-fold, 5000-fold, 10,000-fold, 100,000-fold or more, or any fold change intermediate to these values.

In some embodiments, an “active” cgRNA is said to be “inactivated” by a cognate RNA trigger if the trigger decreases the cgRNA-mediated function of a Cas protein effector on a target gene by 20%, 50%, 90%, 95%, 99%, 99.9%, 99.99%, 99.999%, or 100%, or any percentage intermediate to the stated values. In some embodiments, an “active” cgRNA is said to be “inactivated” by a cognate RNA trigger if the trigger decreases the cgRNA-mediated function of a Cas protein effector on a target gene by 1.5-fold, 2-fold, 5-fold, 10-fold, 20-fold, 50-fold, 100-fold, 200-fold, 500-fold, 1000-fold, 2000-fold, 5000-fold, 10,000-fold, 100,000-fold or more, or any fold change intermediate to these values.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive. Unless defined otherwise, technical and scientific terms used herein have the same meaning as commonly understood by one of ordinary skill in the art to which this invention belongs. See, for example Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). It is to be understood that both the general description and the detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety. Also, the term “embodiment” as used herein refers to an aspect or an implementation of what is disclosed herein, and embodiments may be combined with one another.

Allosteric ON→OFF Terminator Switch cgRNAs (Mechanism 1a)

In some embodiments, allosteric ON→OFF cgRNA logic (FIG. 4A) is implemented using an allosteric ON→OFF terminator switch cgRNA mechanism (FIGS. 4B and 4C). The ON→OFF terminator switch cgRNA of FIGS. 4B and 4C is conditionally inactivated by RNA trigger X (the cognate RNA trigger). Compared to a standard gRNA (FIG. 1B), in some embodiments the ON→OFF terminator switch cgRNA has a modified terminator region with an extended loop and rationally designed sequence domains “d-e-f”. In some embodiments, the cgRNA comprises a target-binding region (domain “u”), a Cas handle, a trigger-binding region (domains “d-e-f′), a first terminator hairpin, and a linker 5′-adjacent to the first terminator hairpin (domain “d”); wherein the first terminator hairpin comprises a 5′ portion of the stem (domain “e”), a 3′ portion of the stem (domain “e*”), and an extended loop (domain “f”). In some embodiments, to toggle to the OFF state, hybridization of the RNA trigger X (the cognate RNA trigger) to the trigger-binding region of the cgRNA (forming the cgRNA:trigger complex) disrupts the structure of the first terminator hairpin to form a structure incompatible with cgRNA mediation of Cas9, dCas9, and/or Cas function (FIG. 10A). In some embodiments, the mechanism is allosteric because the trigger down-regulates cgRNA:Cas function not by sequestering the target-binding region (domain “u” in FIGS. 4B and 4C), but by hybridizing to the distal trigger-binding region (domains “d-e-f′ in FIGS. 4B and 4C). Hence, the sequences of the RNA trigger X and the regulatory target Y (the target gene) are fully independent. In some embodiments, domain “d” in the cgRNA is constrained to a wild-type subsequence of the standard gRNA. In some embodiments, domain “e” in the cgRNA is constrained to a wild-type subsequence of the standard gRNA. In some embodiments, a partial subsequence of domain “f” in the cgRNA is constrained to a wild-type subsequence of the standard gRNA. In some embodiments, domain “d*” in the trigger is optional. In some embodiments, domain “e*” in the trigger is optional. In some embodiments, the extended terminator loop comprises or comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values.

Allosteric OFF→ON Terminator Switch cgRNAs (Mechanism 1B)

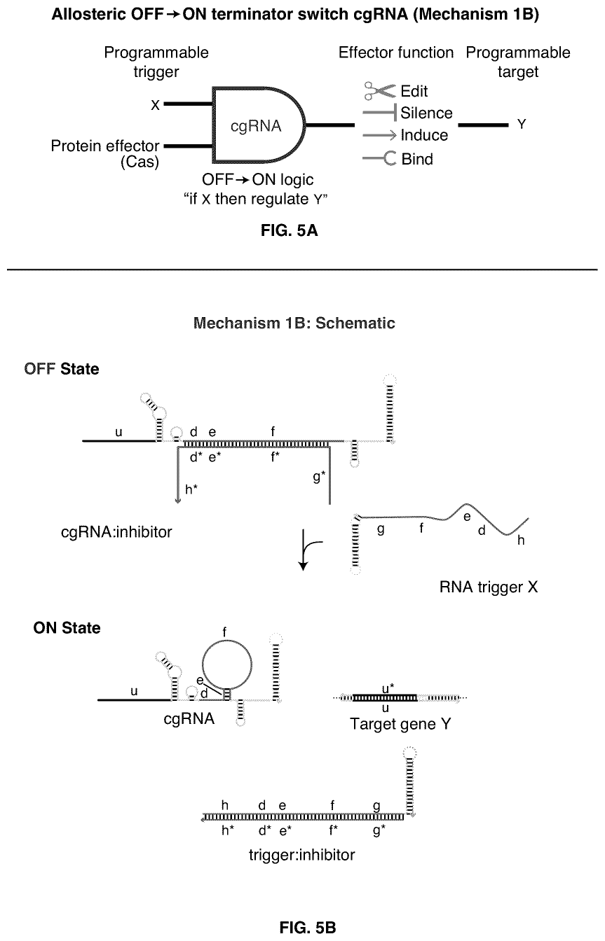

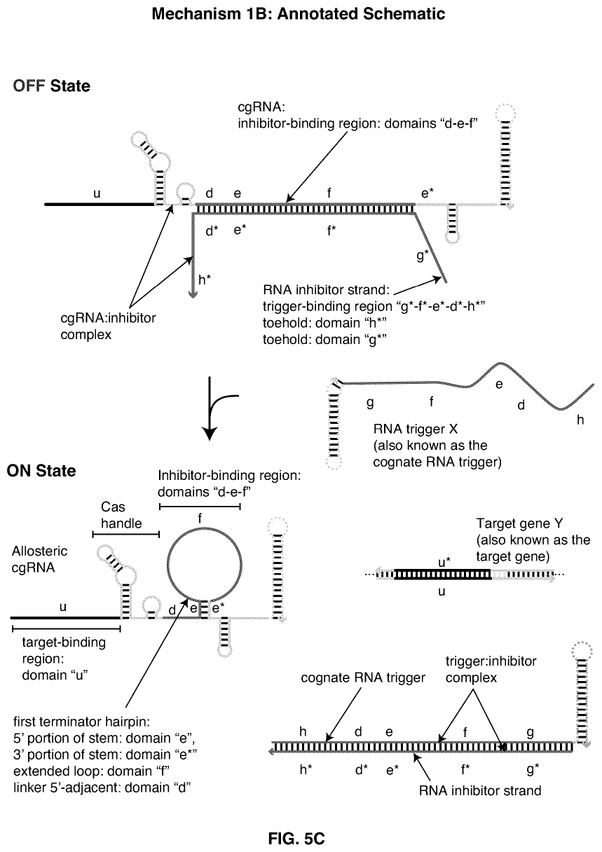

In some embodiments, allosteric OFF→ON cgRNA logic (FIG. 5A) is implemented using an allosteric OFF→ON terminator switch cgRNA mechanism (FIGS. 5B and 5C). The OFF→ON terminator switch cgRNA of FIGS. 5B and 5C is conditionally activated by RNA trigger X (the cognate RNA trigger), which binds to the inhibitor to remove the inhibitor from the cgRNA. Compared to a standard gRNA (FIG. 1B), in some embodiments the OFF→ON terminator switch cgRNA has a modified terminator region with an extended loop and rationally designed sequence domains “d-e-f”. In some embodiments, the cgRNA comprises a target-binding region (domain “u”), a Cas handle, an inhibitor-binding region (domains “d-e-f”), a first terminator hairpin, and a linker 5′-adjacent to the first terminator hairpin (domain “d”); wherein the first terminator hairpin comprises a 5′ portion of the stem (domain “e”), a 3′ portion of the stem (domain “e*”), and an extended loop (domain “f”). In some embodiments, the RNA inhibitor strand comprises a trigger-binding region (domains “g*-f*-e*-d*-h*”) and a toehold at one or both ends (domains “g*” and/or “h*”). In some embodiments, in the OFF state, the inhibitor is hybridized to the inhibitor-binding region of the cgRNA (forming the cgRNA:inhibitor complex) to disrupt the structure of the first terminator hairpin and form a structure incompatible with cgRNA mediation of Cas9, dCas9, and/or Cas function. In some embodiments, to toggle the cgRNA to the ON state, the RNA trigger X (the cognate RNA trigger) displaces the inhibitor from the cgRNA via toehold-mediated strand displacement in which the trigger first nucleates with the inhibitor by binding to the exposed toehold domain “g*” on the inhibitor, and then hybridizes to domains “f*-e*-d*-h*” to displace the inhibitor from the cgRNA (forming the trigger:inhibitor complex). In some embodiments, to toggle the cgRNA to the ON state, the RNA trigger X (the cognate RNA trigger) displaces the inhibitor from the cgRNA via toehold-mediated strand displacement in which the trigger first nucleates with the inhibitor by binding to the exposed toehold domain “h*” on the inhibitor, and then hybridizes to domains “d*”, “e*”, “f*”, and “g*” to displace the inhibitor from the cgRNA (forming the trigger:inhibitor complex). In some embodiments, domain “g” in the trigger is optional. In some embodiments, domain “g*” in the inhibitor is optional. In some embodiments, domain “h” in the trigger is optional. In some embodiments, domain “h*” in the inhibitor is optional. In some embodiments, the mechanism is allosteric because the inhibitor down-regulates cgRNA:Cas function not by sequestering the target-binding region (domain “u” in FIGS. 5B and 5C), but by hybridizing to the distal terminator region comprising domains “d-e-f” in FIGS. 5B and 5C. As a result, the sequence of the RNA trigger X (which binds to the inhibitor to up-regulate cgRNA:Cas function) is independent of domain “u”, yielding full sequence independence between trigger X and regulatory target Y (the target gene). In some embodiments, the extended terminator loop comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values. In some embodiments, a toehold domain comprises, consists, or consists essentially of 4 nt, or 10 nt, or 20 nt, or 30 nt, or 40 nt, or 50 nt, or 100 nt, or 200 nt, or more, or any number of nucleotides intermediate to any of the preceding values.

Allosteric ON→OFF Splinted Switch cgRNAs (Mechanism 2a)

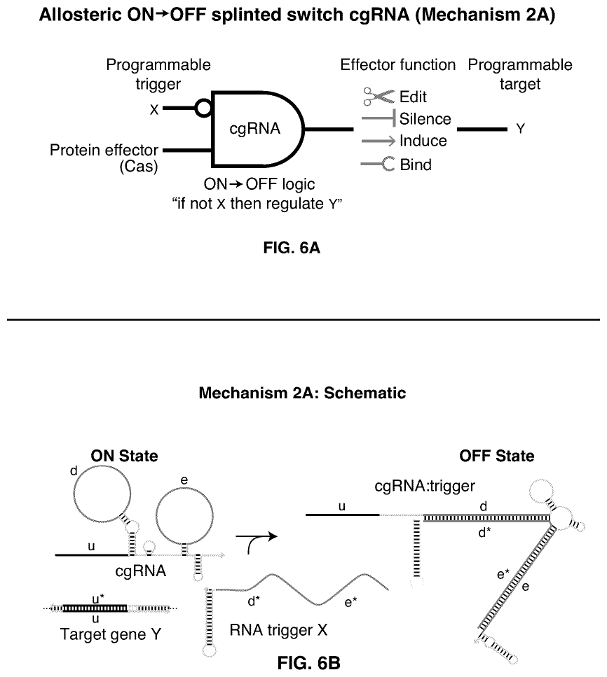

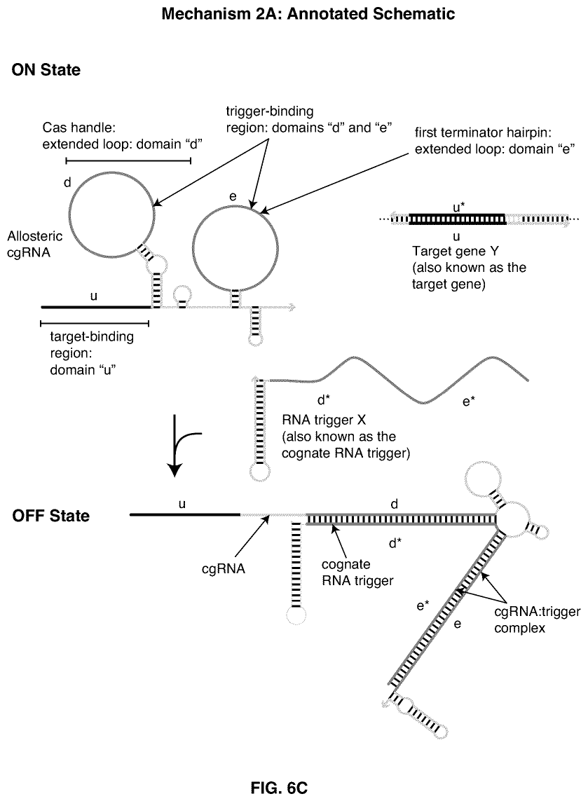

In some embodiments, allosteric ON→OFF cgRNA logic (FIG. 6A) is implemented using an allosteric ON→OFF splinted switch cgRNA mechanism (FIGS. 6B and 6C). The ON→OFF splinted switch cgRNA of FIGS. 6B and 6C is conditionally inactivated by RNA trigger X (the cognate RNA trigger). Compared to a standard gRNA (FIG. 1B), in some embodiments the ON→OFF splinted switch cgRNA has extended loops in both the Cas9 handle (domain “d”) and terminator (domain “e”). In some embodiments, the cgRNA comprises a target-binding region (domain “u”), a Cas handle with an extended loop (domain “d”), a trigger binding region (domains “d” and “e”), and a first terminator hairpin with an extended loop (domain “e”). In some embodiments, to toggle to the OFF state, hybridization of RNA trigger X (the cognate RNA trigger) to the trigger-binding region of the cgRNA (forming the cgRNA:trigger complex) disrupts the structure of the Cas handle and the first terminator hairpin to form a structure incompatible with cgRNA mediation of Cas9, dCas9, and/or Cas function (FIG. 10B). In some embodiments, the mechanism is allosteric because the trigger down-regulates cgRNA:Cas function by hybridizing to extended loops (domains “d” and “e” in FIGS. 6B and 6C) distal to the target-binding region (domain “u” in FIGS. 6B and 6C). The resulting full sequence independence between RNA trigger X (the cognate RNA trigger) and target gene Y (the target gene) provides the flexibility for X to control regulatory scope (also known as the scope of activity) independent of the choice of Y. In some embodiments, the extended Cas handle comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values. In some embodiments, the extended terminator loop comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values.

Allosteric OFF→ON Splinted Switch cgRNAs (Mechanism 2B)

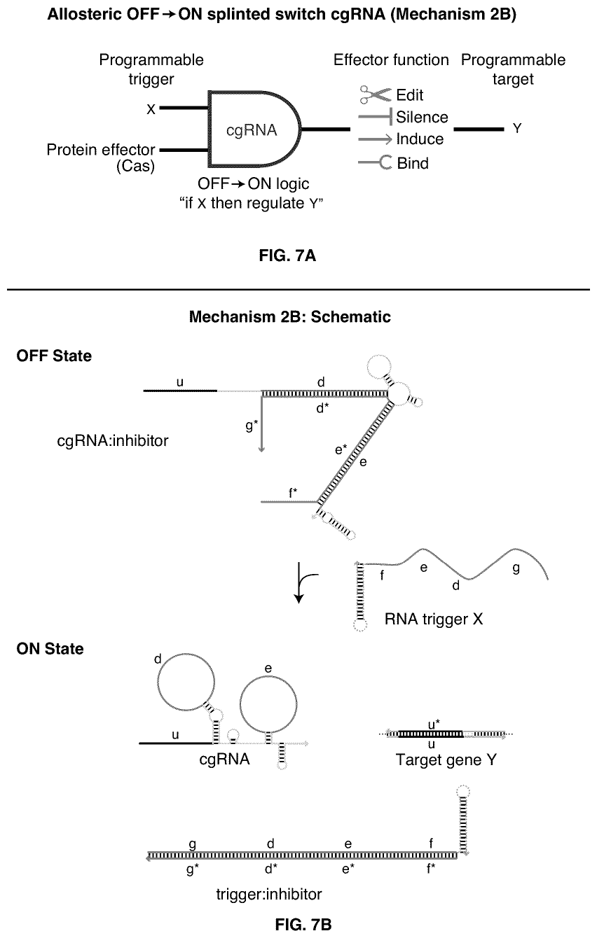

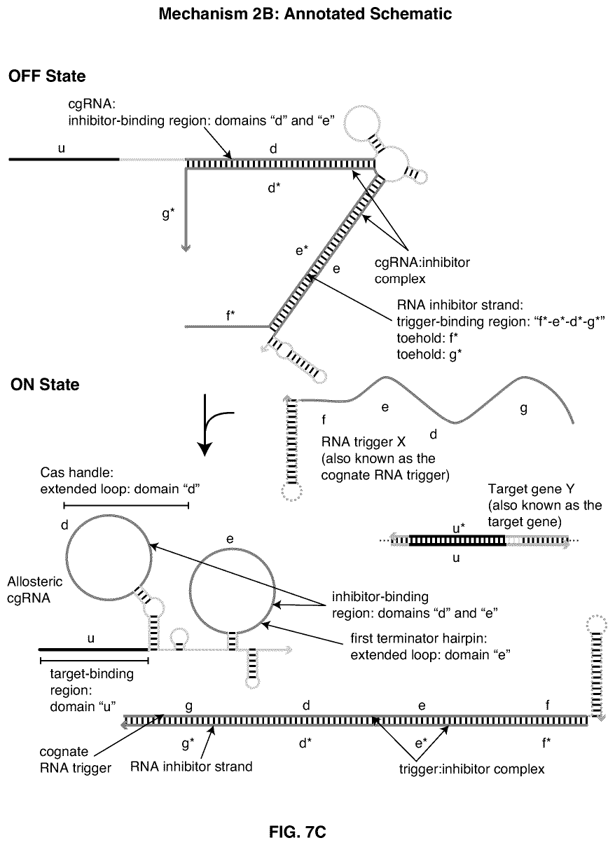

In some embodiments, allosteric OFF→ON cgRNA logic (FIG. 7A) is implemented using an allosteric OFF→ON splinted switch cgRNA mechanism (FIGS. 7B and 7C). The OFF→ON splinted switch cgRNA of FIGS. 7B and 7C is conditionally activated by RNA trigger X (the cognate RNA trigger), which binds to the inhibitor to remove the inhibitor from the cgRNA. Compared to a standard gRNA (FIG. 1B), in some embodiments, the OFF→ON splinted switch cgRNA has extended loops in both the Cas9 handle (domain “d”) and terminator (domain “e”). In some embodiments, the cgRNA comprises a target-binding region (domain “u”), a Cas handle with an extended loop (domain “d”), an inhibitor-binding region (domains “d” and “e”), and a first terminator hairpin with an extended loop (domain “e”). In some embodiments, the RNA inhibitor strand comprises a trigger-binding region (domains “f*-e*-d*-g*”) and a toehold at one or both ends (domains “f*” and/or “g*”). In some embodiments, in the OFF state, the inhibitor is hybridized to the inhibitor-binding region of the cgRNA (forming the cgRNA:inhibitor complex) to disrupt the structure of the Cas handle and the first terminator hairpin and form a structure incompatible with cgRNA mediation of Cas9, dCas9, and/or Cas function. In some embodiments, to toggle the cgRNA to the ON state, the RNA trigger X (the cognate RNA trigger) displaces the inhibitor from the cgRNA via toehold-mediated strand displacement in which the trigger first nucleates with the inhibitor by binding to the exposed toehold domain “f*” on the inhibitor, and then hybridizes to domains “e*-d*-g*” to displace the inhibitor from the cgRNA (forming the trigger:inhibitor complex). In some embodiments, to toggle the cgRNA to the ON state, the RNA trigger X (the cognate RNA trigger) displaces the inhibitor from the cgRNA via toehold-mediated strand displacement in which the trigger first nucleates with the inhibitor by binding to the exposed toehold domain “g*” on the inhibitor, and then hybridizes to domains “d*”, “e*”, and “f*” to displace the inhibitor from the cgRNA (forming the trigger:inhibitor complex). In some embodiments, domain “f” in the trigger is optional. In some embodiments, domain “f*” in the inhibitor is optional. In some embodiments, domain “g” in the trigger is optional. In some embodiments, domain “g*” in the inhibitor is optional. In some embodiments, the mechanism is allosteric because the inhibitor down-regulates cgRNA:Cas function by hybridizing to extended loops (domains “d” and “e” in FIGS. 7B and 7C) distal to the target-binding region (domain “u” in FIGS. 7B and 7C). As a result, the sequence of the RNA trigger X (which binds to the inhibitor to up-regulate cgRNA:Cas function) is independent of domain “u”, yielding full sequence independence between trigger X (the cognate RNA trigger) and regulatory target Y (the target gene). In some embodiments, the extended Cas handle comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values. In some embodiments, the extended terminator loop comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values. In some embodiments, a toehold domain comprises, consists, or consists essentially of 4 nt, or 10 nt, or 20 nt, or 30 nt, or 40 nt, or 50 nt, or 100 nt, or 200 nt, or more, or any number of nucleotides intermediate to any of the preceding values.

Allosteric ON→OFF Tandem Switch cgRNAs (Mechanism 3a)

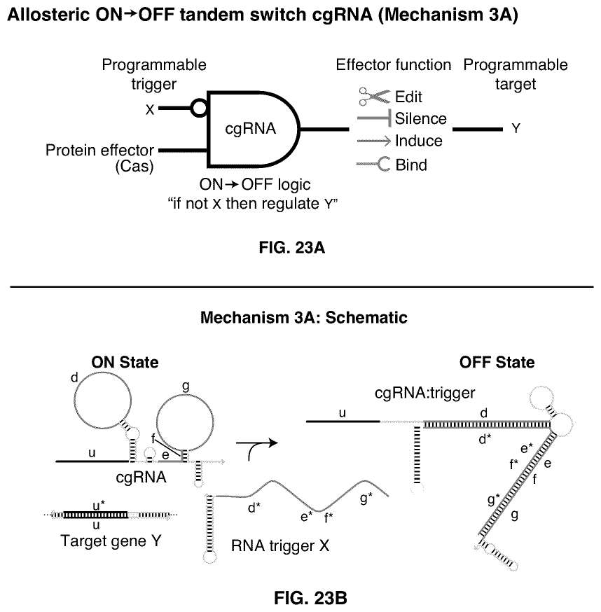

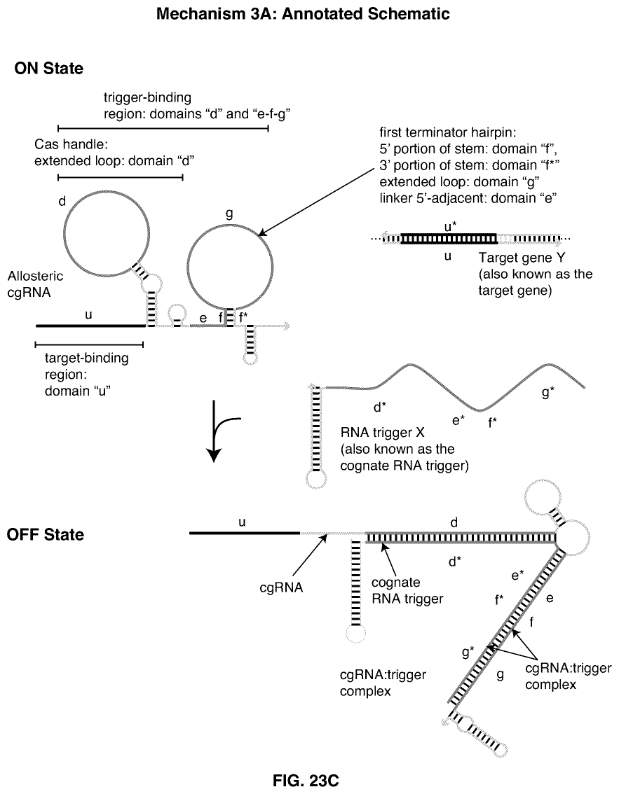

In some embodiments, allosteric ON→OFF cgRNA logic (FIG. 23A) is implemented using an allosteric ON→OFF tandem switch cgRNA mechanism (FIGS. 23B and 23C). The ON→OFF terminator switch cgRNA of FIGS. 23B and 23C is conditionally inactivated by RNA trigger X (the cognate RNA trigger). Compared to a standard gRNA (FIG. 1B), in some embodiments the ON→OFF tandem switch cgRNA has an extended loop in the Cas9 handle (domain “d”) and a modified terminator region with an extended loop and rationally designed sequence domains “e-f-g”. In some embodiments, the cgRNA comprises a target-binding region (domain “u”), a Cas handle with an extended loop (domain “d”), a trigger-binding region (domain “d” and “e-f-g”), a first terminator hairpin, and a linker 5′-adjacent to the first terminator hairpin (domain “e”); wherein the first terminator hairpin comprises a 5′ portion of the stem (domain “f”), a 3′ portion of the stem (domain “f*”), and an extended loop (domain “g”). In some embodiments, to toggle to the OFF state, hybridization of the RNA trigger X (the cognate RNA trigger) to the trigger-binding region of the cgRNA (forming the cgRNA:trigger complex) disrupts the structure of the Cas handle and the first terminator hairpin to form a structure incompatible with cgRNA mediation of Cas9, dCas9, and/or Cas function. In some embodiments, the mechanism is allosteric because the trigger down-regulates cgRNA:Cas function not by sequestering the target-binding region (domain “u” in FIGS. 23B and 23C), but by hybridizing to the distal trigger-binding region (domain “d” and domains “e-f-g” in FIGS. 23B and 23C). Hence, the sequences of the RNA trigger X (the cognate RNA trigger) and the regulatory target Y (the target gene) are fully independent. In some embodiments, domain “e” in the cgRNA is constrained to a wild-type subsequence of the standard gRNA. In some embodiments, domain “f′ in the cgRNA is constrained to a wild-type subsequence of the standard gRNA. In some embodiments, a partial subsequence of domain “g” in the cgRNA is constrained to a wild-type subsequence of the standard gRNA. In some embodiments, a partial subsequence of domain “d” in the cgRNA is constrained to a wild-type subsequence of the standard gRNA. In some embodiments, domain “e*” in the trigger is optional. In some embodiments, domain “f*” in the trigger is optional. In some embodiments, the extended Cas handle comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values. In some embodiments, the extended terminator loop comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values.

Allosteric OFF→ON Tandem Switch cgRNAs (Mechanism 3B)

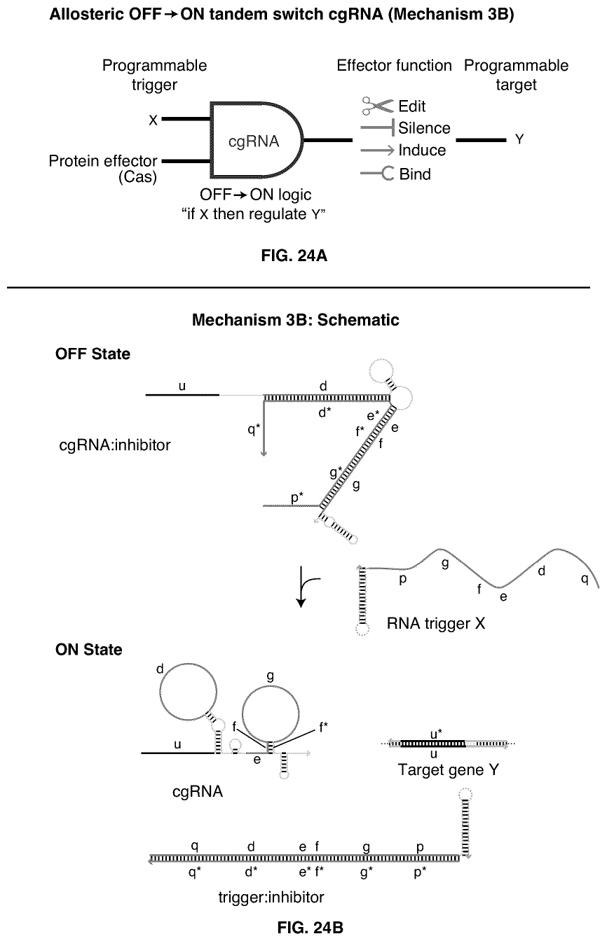

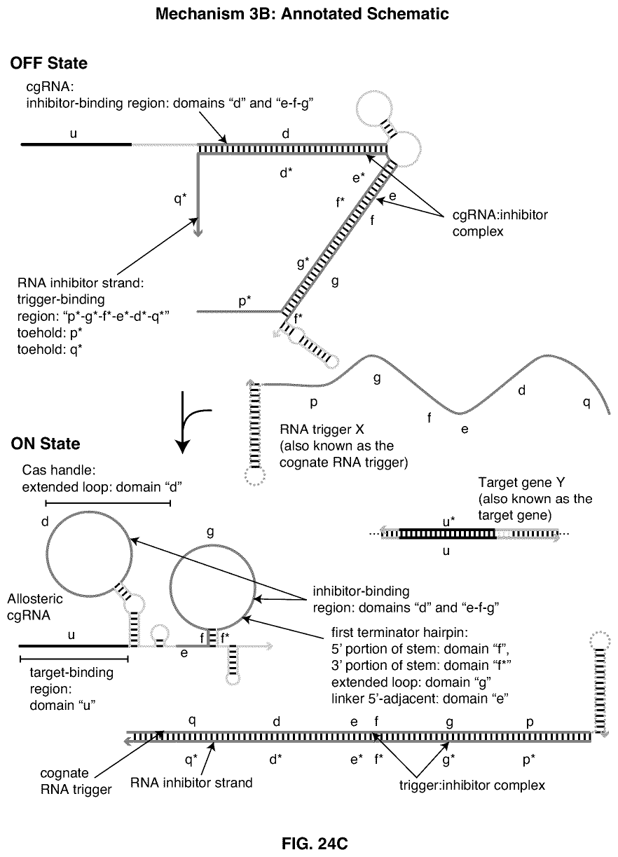

In some embodiments, allosteric OFF→ON cgRNA logic (FIG. 24A) is implemented using an allosteric OFF→ON tandem switch cgRNA mechanism (FIGS. 24B and 24C). The OFF→ON tandem switch cgRNA of FIGS. 24B and 24C is conditionally activated by RNA trigger X (the cognate RNA trigger), which binds to the inhibitor to remove the inhibitor from the cgRNA. Compared to a standard gRNA (FIG. 1B), in some embodiments the OFF→ON tandem switch cgRNA has an extended loop in the Cas9 handle (domain “d”) and a modified terminator region with an extended loop and rationally designed sequence domains “e-f-g”. In some embodiments, the cgRNA comprises a target-binding region (domain “u”), a Cas handle with an extended loop (domain “d”), an inhibitor-binding region (domains “d” and “e-f-g”), a first terminator hairpin, and a linker 5′-adjacent to the first terminator hairpin (domain “e”); wherein the first terminator hairpin comprises a 5′ portion of the stem (domain “f”), a 3′ portion of the stem (domain “f*”), and an extended loop (domain “g”). In some embodiments, the RNA inhibitor strand comprises a trigger-binding region (domains “p*-g*-f*-e*-d*-q*”) with a toehold at one or both ends (domains “p*” and/or “q*”). In the OFF state, the inhibitor is hybridized to the inhibitor-binding region of the cgRNA (forming the cgRNA:inhibitor complex) to disrupt the structure of the Cas handle and the first terminator hairpin and form a structure incompatible with cgRNA mediation of Cas9, dCas9, and/or Cas function. In some embodiments, to toggle the cgRNA to the ON state, the RNA trigger X (the cognate RNA trigger) displaces the inhibitor from the cgRNA via toehold-mediated strand displacement in which the trigger first nucleates with the inhibitor by binding to the exposed toehold domain “p*” on the inhibitor, and then hybridizes to domains “g*-f*-e*-d*-q*” to displace the inhibitor from the cgRNA (forming the trigger:inhibitor complex). In some embodiments, to toggle the cgRNA to the ON state, the RNA trigger X (the cognate RNA trigger) displaces the inhibitor from the cgRNA via toehold-mediated strand displacement in which the trigger first nucleates with the inhibitor by binding to the exposed toehold domain “q*” on the inhibitor, and then hybridizes to domains “d*”, “e*”, “f*”, “g*”, and “p*” to displace the inhibitor from the cgRNA (forming the trigger:inhibitor complex). In some embodiments, domain “p” in the trigger is optional. In some embodiments, domain “p*” in the inhibitor is optional. In some embodiments, domain “q” in the trigger is optional. In some embodiments, domain “q*” in the inhibitor is optional. In some embodiments, the mechanism is allosteric because the inhibitor down-regulates cgRNA:Cas function not by sequestering the target-binding region (domain “u” in FIGS. 24B and 24C), but by hybridizing to the distal terminator region comprising domains “d” and “e-f-g” in FIGS. 24B and 24C. As a result, the sequence of the RNA trigger X (which binds to the inhibitor to up-regulate cgRNA:Cas function) is independent of domain “u”, yielding full sequence independence between trigger X (the cognate RNA trigger) and regulatory target Y (the target gene). In some embodiments, the extended Cas handle comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values. In some embodiments, the extended terminator loop comprises, consists, or consists essentially of 4 nt, or 5 nt, or 6 nt, or 8 nt, or 10 nt, or 20 nt, or 30 nt, or 40 nt, or 50 nt, or 100 nt, or 200 nt, or more, or any number of nucleotides intermediate to any of the preceding values. In some embodiments, a toehold domain comprises, consists, or consists essentially of 4 nt, or 10 nt, or 20 nt, or 30 nt, or 40 nt, or 50 nt, or 100 nt, or 200 nt, or more, or any number of nucleotides intermediate to any of the preceding values.

Allosteric OFF→ON Split-Terminator Switch cgRNAs (Mechanism 4a)

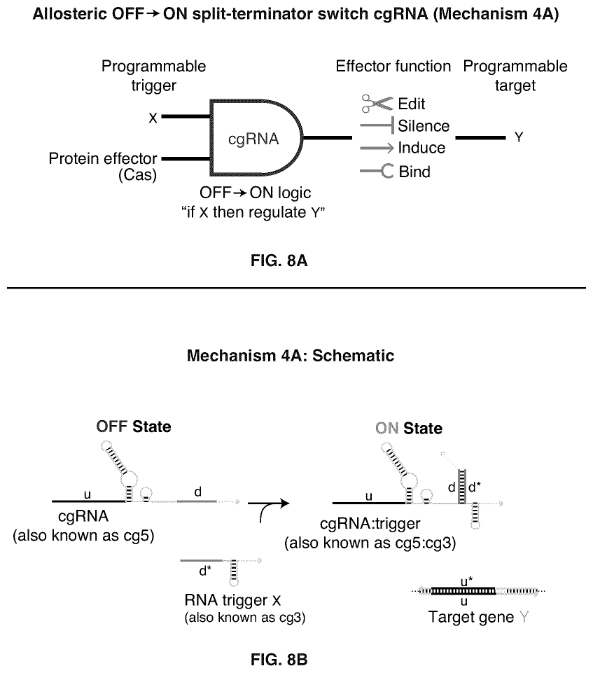

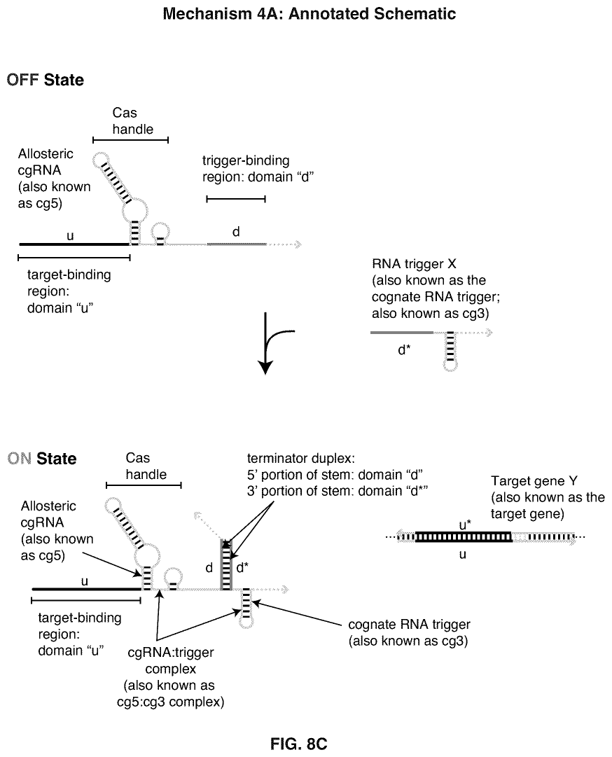

In some embodiments, allosteric OFF→ON cgRNA logic (FIG. 8A) is implemented using an allosteric OFF→ON split-terminator switch cgRNA mechanism (FIGS. 8B and 8C). The OFF→ON split-terminator switch cgRNA of FIGS. 8B and 8C is conditionally activated by RNA trigger X (the cognate RNA trigger). Equivalently, the cgRNA may be interpreted as a 5′ fragment (cg5) and the trigger may be interpreted as a 3′ fragment (cg3), such that cg5 and cg3 are inactive when not bound to each other, but such that upon binding to each other to form the complex cg5:cg3, this complex constitutes an activated conditional guide RNA capable of mediating Cas9, dCas9, and/or Cas function. Compared to a standard gRNA (FIG. 1B), in some embodiments the OFF→ON split-terminator switch cgRNA (also known as cg5) is incomplete, containing only one half of the stem region of the 5′ terminator hairpin (sequence domain “d”). In some embodiments, the cgRNA (also known as cg5) comprises a target-binding region (domain “u”), a Cas handle, and a trigger-binding region (domain “d”). In some embodiments, to toggle to the ON state, hybridization of the RNA trigger X (the cognate RNA trigger; also known as cg3) to the trigger-binding region of the cgRNA (also known as cg5) to form the cgRNA:trigger complex (also known as the cg5:cg3 complex) yields a terminator duplex that activates the cgRNA (or equivalently yields a terminator duplex that activates the complex cg5:cg3), allowing for mediation of Cas function (FIG. 10C). In some embodiments, the terminator duplex comprises a 5′ portion of the stem (domain “d”) and a 3′ portion of the stem (domain “d*”). In some embodiments, the mechanism is allosteric because the trigger (also known as cg3) and cgRNA (also known as cg5) interact via a terminator duplex (domains “d” and “d*” in FIGS. 8B and 8C) distal to the target-binding region (domain “u” in FIGS. 8B and 8C). The resulting full sequence independence between RNA trigger X (the cognate RNA trigger; also known as cg3) and target gene Y (the target gene) provides the flexibility for X to control regulatory scope (also known as the scope of activity) independent of the choice of Y. In some embodiments, the terminator duplex comprises, consists, or consists essentially of 2 bp, or 4 bp, or 6 bp, or 8 bp, or 10 bp, or 20 bp, or 30 bp, or 40 bp, or 50 bp, or 100 bp, or 200 bp, or more, or any number of base pairs intermediate to any of the preceding values.

In some embodiments, the cgRNA (also known as cg5) comprises one or more substrate domains 5′-adjacent to domain “d” and the cognate RNA trigger (also known as cg3) comprises one or more substrate domains 5′-adjacent to domain “d*”. In some embodiments, an additional bridge strand hybridizes to a substrate on the cgRNA (also known as cg5) and to a substrate on the helper strand (also known as cg3) for the purpose of holding the cgRNA (also known as cg5) and helper strand (also known as cg3) together in a complex (cg5:cg3: bridge). In some embodiments, an inhibitor strand hybridizes to one or more substrates on the cgRNA (also known as cg5) and one or more substrates on the cognate RNA trigger (also known as cg3) so as to form a junction that disrupts the structure of the terminator duplex, wherein the structure of the cgRNA:trigger:inhibitor complex (also known as the cg5:cg3:inhibitor complex) is incompatible with mediation of Cas9, dCas9, and/or Cas function. In some embodiments, the cgRNA (also known as cg5) can be activated (OFF→ON logic) by the trigger (also known as cg3) and then later inactivated by the inhibitor (ON→OFF logic) corresponding overall to (OFF→ON→OFF) logic. In some embodiments, a cgRNA can be activated and then subsequently inactivated. In some embodiments, the activity of a cgRNA can be repeatedly toggled between the OFF and ON states by successive interactions with a first trigger, a first inhibitor, a second trigger, a second inhibitor, and so on.

Allosteric ON→OFF Split-Terminator Switch cgRNAs (Mechanism 4B)

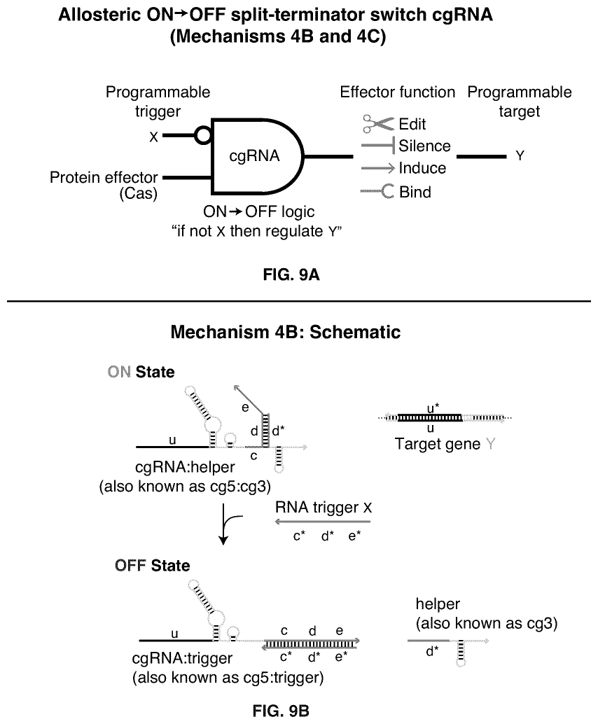

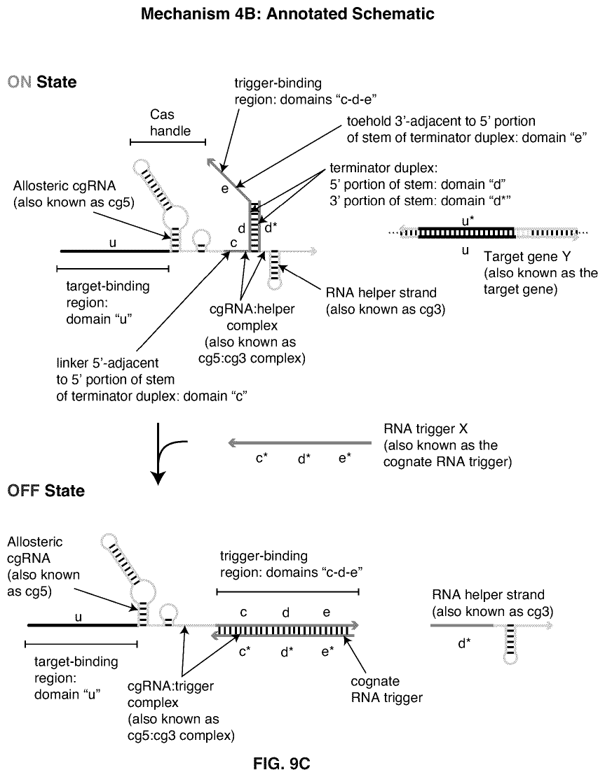

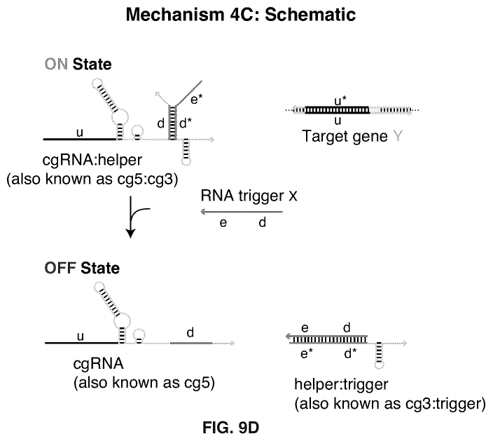

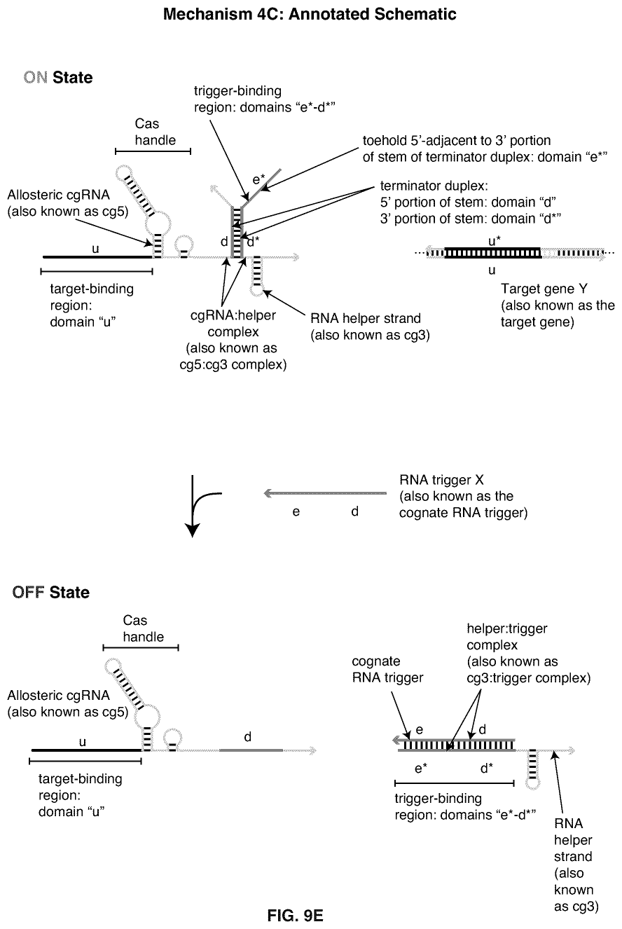

In some embodiments, allosteric ON→OFF cgRNA logic (FIG. 9A) is implemented using an allosteric ON→OFF split-terminator switch cgRNA mechanism (FIGS. 9B and 9C). In some embodiments, the ON→OFF split-terminator switch cgRNA (also known as cg5) of FIGS. 9B and 9C is conditionally inactivated by RNA trigger X (cognate RNA trigger), which binds to the cgRNA to displace the RNA helper strand (also known as cg3) and form a structure incompatible with cgRNA mediation of Cas9, dCas9, and/or Cas function. Equivalently, in some embodiments, the functional conditional guide RNA can be interpreted as a complex of two fragments (complex cg5:cg3 comprising 5′-fragment cg5 and 3′-fragment cg3; FIGS. 9B and 9C) that is conditionally inactivated by RNA trigger X (cognate RNA trigger), which binds to the complex to displace cg3 and form complex cg5:trigger, wherein both cg5:trigger and cg3 are structurally incompatible with mediation of Cas9, dCas9, and/or Cas function. Compared to a standard gRNA (FIG. 1B), in some embodiments the ON→OFF split-terminator switch cgRNA (also known as cg5) is incomplete, containing only one half of the stem region of the 5′ terminator hairpin (sequence domain “d”). In some embodiments, in the ON state, hybridization of the RNA helper strand (also known as cg3) to domain “d” forms a terminator duplex (domain “d” base-paired to domain “d*”) such that the cgRNA: helper complex (also known as the cg5:cg3 complex) is active. In some embodiments, the cgRNA (also known as cg5) comprises a target-binding region (domain “u”), a Cas handle, and a trigger-binding region (domains “c-d-e”); wherein the trigger-binding region comprises a 5′ portion of the stem of the terminator duplex (domain “d”), a linker 5′-adjacent to the 5′ portion of the stem of the terminator duplex (domain “c”), and a toehold 3′-adjacent to the 5′ portion of the stem of the terminator duplex (domain “e”). In some embodiments, the RNA helper strand (also known as cg3) comprises a 3′ portion of the stem of the terminator duplex (domain “d*”). In some embodiments, to toggle the cgRNA (also known as cg5) to the OFF state, the RNA trigger X (cognate RNA trigger) displaces the helper (also known as cg3) from the cgRNA (also known as cg5) via toehold-mediated strand displacement in which the trigger first nucleates with the cgRNA (also known as cg5) by binding to the exposed toehold domain “e” on the cgRNA, and then hybridizes to domains “d” and “c” to displace the helper (also known as cg3) from the cgRNA (forming the cgRNA:trigger complex; also known as the cg5:trigger complex), yielding a structure with a disrupted linker (domain “c”) and lacking the remainder of the terminator region present in the RNA helper strand (also known as cg3), wherein the structure is incompatible with cgRNA mediation of Cas9, dCas9, and/or Cas function. In some embodiments, to toggle the cgRNA to the OFF state, the RNA trigger X (cognate RNA trigger) displaces the helper (also known as cg3) from the cgRNA (also known as cg5) via toehold-mediated strand displacement in which the trigger first nucleates with the cgRNA (also known as cg5) by binding to the exposed toehold domain “c” (also known as the linker domain “c”) on the cgRNA (also known as cg5), and then hybridizes to domains “d” and “e” to displace the helper (also known as cg3) from the cgRNA, yielding a cgRNA:trigger complex (also known as the cg5:trigger complex) that is structurally incompatible with cgRNA mediation of Cas9, dCas9, and/or Cas function. In some embodiments, the mechanism is allosteric because the cgRNA and helper interact via a terminator duplex (domains “d” and “d*” in FIGS. 9B and 9C) distal to the target-binding region (domain “u” in FIGS. 9B and 9C). As a result, the sequence of the RNA trigger X (which binds to the cgRNA domains “c-d-e” to down-regulate Cas function) is also independent of domain “u”, yielding full sequence independence between trigger X (the cognate RNA trigger) and regulatory target Y (the target gene). In some embodiments, domain “c*” in the trigger is optional. In some embodiments, domain “e*” in the trigger is optional. In some embodiments, domain “e” in the cgRNA is optional. In some embodiments, domain “c” in the cgRNA is constrained to a wild-type subsequence of the standard gRNA. In some embodiments, the terminator duplex comprises, consists, or consists essentially of 2 bp, or 4 bp, 6 bp, or 8 bp, or 10 bp, or 20 bp, or 30 bp, or 40 bp, or 50 bp, or 100 bp, or 200 bp, or more, or any number of base pairs intermediate to any of the preceding values. In some embodiments, a toehold domain comprises, consists, or consists essentially of 4 nt, or 10 nt, or 20 nt, or 30 nt, or 40 nt, or 50 nt, or 100 nt, or 200 nt, or more, or any number of nucleotides intermediate to any of the preceding values.

Allosteric ON→OFF Split-Terminator Switch cgRNAs (Mechanism 4C)

In some embodiments, allosteric ON→OFF cgRNA logic (FIG. 9A) is implemented using an allosteric ON→OFF split-terminator switch cgRNA mechanism (FIGS. 9D and 9E). In some embodiments, the ON→OFF split-terminator switch cgRNA (also known as cg5) of FIGS. 9D and 9E is conditionally inactivated by RNA trigger X (the cognate RNA trigger), which binds to the RNA helper strand (also known as cg3) to remove it from the cgRNA (also known as cg5), which then lacks the terminator duplex and the remainder of the terminator region, yielding a structure incompatible with mediation of Cas9, dCas9, and/or Cas function. Equivalently, in some embodiments, the functional conditional guide RNA can be interpreted as a complex of two fragments (complex cg5:cg3 comprising 5′-fragment cg5 and 3′-fragment cg3; FIGS. 9D and 9E) that is conditionally inactivated by RNA trigger X (cognate RNA trigger), which binds to the complex to displace cg5 and form the cg3:trigger complex, wherein both cg3:trigger and cg5 are structurally incompatible with mediation of Cas9, dCas9, and/or Cas function. Compared to a standard gRNA (FIG. 1B), in some embodiments the ON→OFF split-terminator switch cgRNA (also known as cg5) is incomplete, containing only one half of the stem region of the 5′ terminator hairpin (sequence domain “d”). In the ON state, hybridization of the RNA helper strand (also known as cg3) to domain “d” forms a terminator duplex (domain “d” base-paired to domain “d*”) such that the cgRNA: helper complex (also known as the cg5:cg3 complex) is active. In some embodiments, the cgRNA (also known as cg5) comprises a target-binding region (domain “u”), a Cas handle, and a 5′ portion of the stem of the terminator duplex (domain “d”). In some embodiments, the RNA helper strand (also known as cg3) comprises a trigger-binding region (domains “e*-d*”); wherein the trigger-binding region comprises a 3′ portion of the stem of the terminator duplex (domain “d*”) and a toehold 5′-adjacent to the 3′ portion of the stem of the terminator duplex (domain “e*”). In some embodiments, to toggle the cgRNA (also known as cg5) to the OFF state, the RNA trigger X (the cognate RNA trigger) displaces the RNA helper strand (also known as cg3) from the cgRNA (also known as cg5) via toehold-mediated strand displacement in which the trigger first nucleates with the helper (also known as cg3) by binding to the exposed toehold domain “e*” on the helper (also known as cg3), and then hybridizes to domain “d*” to displace the helper (also known as cg3) from the cgRNA (also known as cg5), forming the helper:trigger complex (also known as the cg3:trigger complex). This yields an incomplete cgRNA (also known as cg5) lacking the terminator duplex, wherein the cgRNA (also known as cg5) is structurally incompatible with mediation of Cas9, dCas9, and or Cas function. In some embodiments, the mechanism is allosteric because the cgRNA (also known as cg5) and helper (also known as cg3) interact via a terminator duplex (domains “d” and “d*” in FIGS. 9D and 9E) distal to the target-binding region (domain “u” in FIGS. 9D and 9E). As a result, the sequence of the RNA trigger X (which binds to the helper domains “e*-d*” to down-regulate Cas function) is independent of domain “u”, yielding full sequence independence between trigger X (the cognate RNA trigger) and regulatory target Y (the target gene). In some embodiments, the terminator duplex comprises, consists, or consists essentially of 2 bp, or 4 bp, 6 bp, or 8 bp, or 10 bp, or 20 bp, or 30 bp, or 40 bp, or 50 bp, or 100 bp, or 200 bp, or more, or any number of base pairs intermediate to any of the preceding values. In some embodiments, a toehold domain comprises, consists, or consists essentially of 4 nt, or 10 nt, or 20 nt, or 30 nt, or 40 nt, or 50 nt, or 100 nt, or 200 nt, or more, or any number of nucleotides intermediate to any of the preceding values.

In some embodiments, the RNA helper strand (also known as cg3) comprises one or more additional domains 5′-adjacent to domain “d*”. In some embodiments, one or more of these additional domains functions as a toehold to nucleate interactions with an auxiliary trigger that hybridizes to the helper strand (also known as cg3) to remove the helper strand (also known as cg3) from the cgRNA (also known as cg5). In some embodiments, one or more of these additional domains functions as a substrate to provide a binding site for an additional strand that hybridizes to both the substrate and to the cgRNA (also known as cg5) for the purpose of holding the cgRNA (also known as cg5) and helper strand (also known as cg3) together in complex (cg5:cg3: additional-strand). In some embodiments, the RNA helper strand (also known as cg3) comprises one or more additional domains 3′-adjacent to domain “d*”. In some embodiments, one or more of these additional domains functions as a toehold to nucleate interactions with an auxiliary trigger that hybridizes to the helper strand (also known as cg3) to remove the helper strand (also known as cg3) from the cgRNA (also known as cg5). In some embodiments, the cgRNA (also known as cg5) comprises one or more substrate domains 3′-adjacent to domain “d” and the RNA helper strand (also known as cg3) comprises one or more substrate domains 5′-adjacent to domain “d*”. In some embodiments, the trigger hybridizes to one or more substrates on the cgRNA (also known as cg5) and one or more substrates on the helper strand (also known as cg3) so as to form a 3-way junction that disrupts the structure of the terminator duplex, wherein the structure of the cgRNA: helper: trigger complex (also known as the cg5:cg3: trigger complex) is incompatible with mediation of Cas9, dCas9, and/or Cas function.

Allosteric OFF→ON 5′-Inhibited Split-Terminator Switch cgRNAs (Mechanism 5)

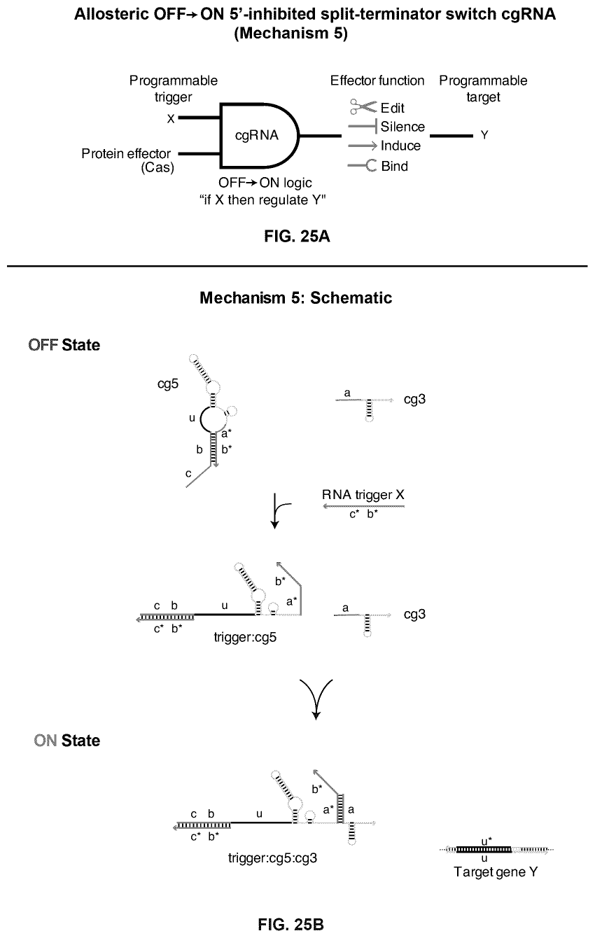

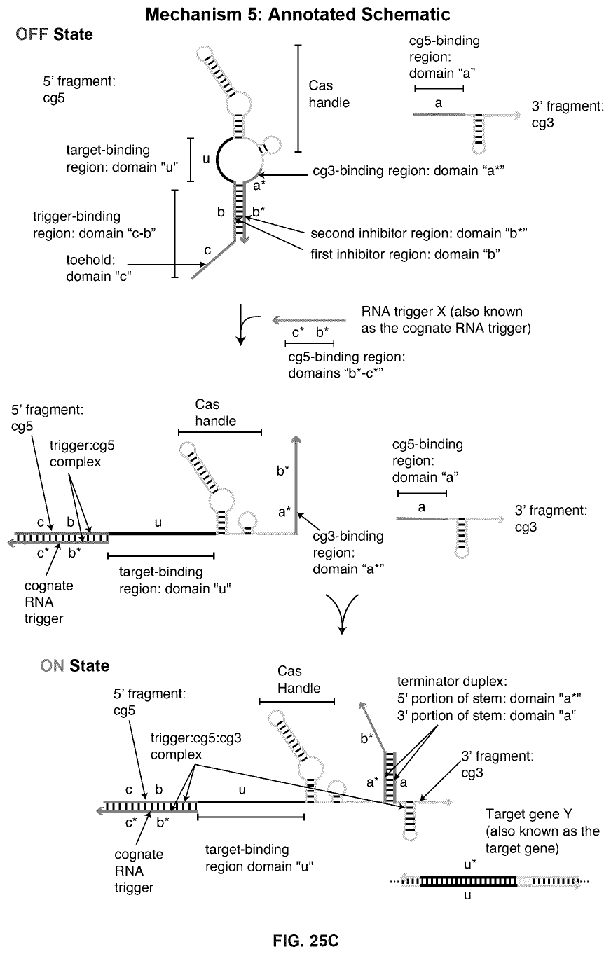

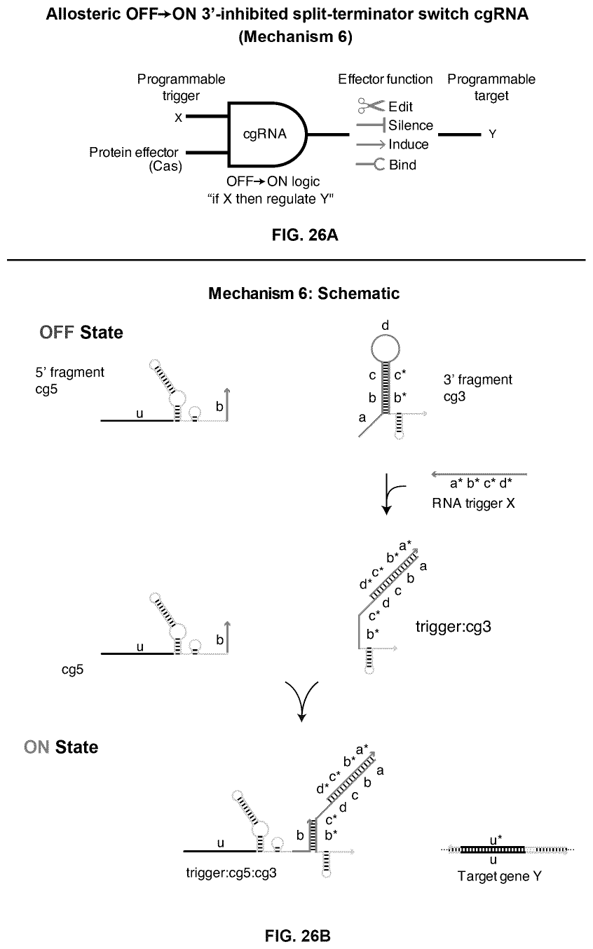

In some embodiments, allosteric OFF→ON cgRNA logic (FIG. 25A) is implemented using an allosteric OFF→ON 5′-inhibited split-terminator switch cgRNA mechanism (FIGS. 25B and 25C). In some embodiments, the OFF→ON 5′-inhibited split-terminator switch cgRNA (FIGS. 25B and 25C) comprises two inactive cgRNA fragments (cg5 and cg3) that are conditionally activated by RNA trigger X (the cognate RNA trigger), which binds to cg5 to allow for formation of the trigger:cg5:cg3 complex that activates the cgRNA, mediating the function of a Cas protein effector on a target gene (Y). In some embodiments, the OFF→ON 5′-sequestered split-terminator switch cgRNA comprises two fragments (FIGS. 25B and 25C), a 5′-fragment (cg5) and a 3′-fragment (cg3), that are inactive when not bound to each other (OFF state). In some embodiments, cg5 comprises a Cas handle, a target-binding region (sequence domain “u”) 5′ of the Cas handle, and a cg3-binding region (sequence domain “a*”) 3′ of the Cas handle. In some embodiments, cg3 comprises a cg5-binding region (sequence domain “a”). In some embodiments, cg5 further comprises a trigger-binding region (sequence domains “c-b”) comprising a first inhibitor region (sequence domain “b”), and a second inhibitor region (sequence domain “b*”), such that in the absence of the cognate RNA trigger, the first inhibitor region (sequence domain “b”) is configured to bind to the second inhibitor region (sequence domain “b*”), inhibiting binding between cg5 and cg3 by sequestering the cg3-binding region of cg5 (sequence domain “a*”) in a loop. In some embodiments, the cognate RNA trigger comprises a cg5-binding region (sequence domains “b*-c*”), the trigger-binding region further comprises a toehold of one or more unpaired nucleotides at one or both ends (for example, domain “c”), the cg3-binding region of cg5 comprises the 5′ portion of the stem of a terminator duplex (sequence domain “d”), and the cg5-binding region of cg3 comprises the 3′ portion of the stem of the terminator duplex (sequence domain “d*”), such that upon hybridization of the cognate RNA trigger to cg5, cg5 hybridizes to cg3 to form the terminator duplex, activating the cgRNA (ON state). In some embodiments, the target-binding region (sequence domain “u”) is non-overlapping with the trigger-binding region (sequence domains “c-b”) and is configured not to bind to the trigger-binding region, allowing for allosteric signal transduction, in which the sequence of the cognate RNA trigger (X) can be selected independently of the sequence of the target gene (Y). In some embodiments, the cognate RNA trigger comprises additional flanking nucleotides 5′ and/or 3′ of the cg5-binding region. In some embodiments, to toggle the cgRNA from the OFF state to the ON state, the RNA trigger X (cognate RNA trigger) opens up the sequestering loop in cg5 via toehold-mediated strand displacement in which the trigger first nucleates with cg5 by hybridizing to the exposed toehold “c” on cg5 and then hybridizes to domain “b” to open the loop containing domain “a*”, facilitating hybridization between domain “a*” in cg5 and domain “a” in cg3 to form the terminator duplex and activate the cgRNA, allowing for mediation of Cas function on the target gene (FIG. 25C). In some embodiments, the mechanism is allosteric because the trigger and cg5 interact via domains “c-b” in cg5 and “b*-c*” in the trigger (see FIGS. 25B and 25C) that are independent of the target-binding region (domain “u” in FIGS. 25B and 25C). The resulting full sequence independence between RNA trigger X (the cognate RNA trigger) and target gene Y (the target gene) provides the flexibility for X to control regulatory scope (also known as the scope of activity) independent of the choice of Y. In some embodiments, the terminator duplex comprises, consists, or consists essentially of 2 bp, or 4 bp, 6 bp, or 8 bp, or 10 bp, or 20 bp, or 30 bp, or 40 bp, or 50 bp, or 100 bp, or 200 bp, or more, or any number of base pairs intermediate to any of the preceding values.

Allosteric OFF→ON 3′-Inhibited Split-Terminator Switch cgRNAs (Mechanism 6)

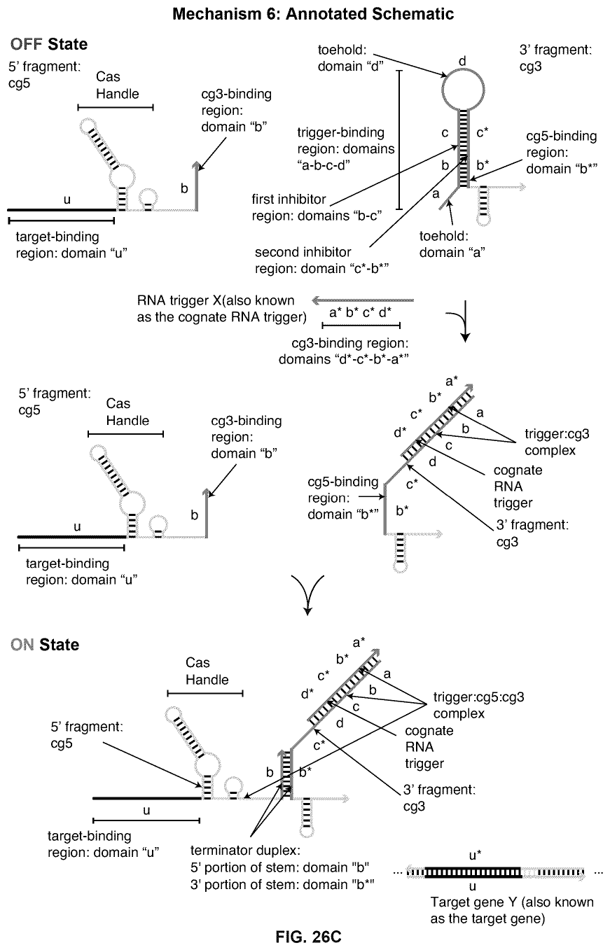

In some embodiments, allosteric OFF→ON cgRNA logic (FIG. 26A) is implemented using an allosteric OFF→ON 3′-inhibited split-terminator switch cgRNA mechanism (FIGS. 26B and 26C). In some embodiments, the OFF→ON 3′-inhibited split-terminator switch cgRNA (FIGS. 26B and 26C) comprises two inactive cgRNA fragments (cg5 and cg3) that are conditionally activated by RNA trigger X (the cognate RNA trigger), which binds to cg3 to allow for formation of the trigger:cg5:cg3 complex that activates the cgRNA, mediating the function of a Cas protein effector on a target gene (Y). In some embodiments, the OFF→ON 3′-sequestered split-terminator switch cgRNA comprises two fragments (FIGS. 26B and 26C), a 5′-fragment (cg5) and a 3′-fragment (cg3), that are inactive when not bound to each other (OFF state). In some embodiments, cg5 comprises a Cas handle, a target-binding region (sequence domain “u”) 5′ of the Cas handle, and a cg3-binding region (sequence domain “b”) 3′ of the Cas handle. In some embodiments, cg3 comprises a cg5-binding region (sequence domain “b*”). In some embodiments, cg3 further comprises a trigger-binding region (sequence domains “a-b-c-d”) comprising a first inhibitor region (sequence domains “b-c”), and a second inhibitor region (sequence domain “c*-b*”), such that in the absence of the cognate RNA trigger, the first inhibitor region (sequence domains “b-c”) is configured to bind to the second inhibitor region (sequence domain “c*-b*”), inhibiting binding between cg3 and cg5 by sequestering the cg5-binding region of cg3 (sequence domain “b*”) in a duplex (i.e., the duplex formed by base-pairing between domains “b” and “b*” within cg3). In some embodiments, the cognate RNA trigger comprises a cg3-binding region (sequence domains “d*-c*-b*-a*”), the trigger-binding region further comprises a toehold of one or more unpaired nucleotides at one or both ends (for example, domain “a” and/or domain “d”), the cg3-binding region of cg5 comprises the 5′ portion of the stem of a terminator duplex (sequence domain “b”), and the cg5-binding region of cg3 comprises the 3′ portion of the stem of the terminator duplex (sequence domain “b*”), such that upon hybridization of the cognate RNA trigger to cg3, cg3 hybridizes to cg5 to form the terminator duplex, activating the cgRNA (ON state). In some embodiments, the target-binding region (sequence domain “u”) is non-overlapping with the trigger-binding region (sequence domains “a-b-c-d”) and is configured not to bind to the trigger-binding region, allowing for allosteric signal transduction, in which the sequence of the cognate RNA trigger (X) can be selected independently of the sequence of the target gene (Y). In some embodiments, the cognate RNA trigger comprises additional flanking nucleotides 5′ and/or 3′ of the cg3-binding region. In some embodiments, to toggle the cgRNA from the OFF state to the ON state, the RNA trigger X (cognate RNA trigger) opens up the duplex in cg3 via toehold-mediated strand displacement in which the trigger first nucleates with cg3 by hybridizing to the exposed toehold “a” on cg3 (and/or the exposed toehold “d”) and then hybridizes to domains “b-c” to open the duplex sequestering domain “b*”, facilitating hybridization between domain “b*” in cg3 and domain “b” in cg5 to form the terminator duplex and activate the cgRNA, allowing for mediation of Cas function on the target gene (FIG. 26C). In some embodiments, the mechanism is allosteric because the trigger and cg3 interact via domains “a-b-c-d” in cg3 and “d*-c*-b*-a*” in the trigger (see FIGS. 26B and 26C) that are independent of the target-binding region (domain “u” in cg5; FIGS. 26B and 26C). The resulting full sequence independence between RNA trigger X (the cognate RNA trigger) and target gene Y (the target gene) provides the flexibility for X to control regulatory scope (also known as the scope of activity) independent of the choice of Y. In some embodiments, the terminator duplex comprises, consists, or consists essentially of 2 bp, or 4 bp, 6 bp, or 8 bp, or 10 bp, or 20 bp, or 30 bp, or 40 bp, or 50 bp, or 100 bp, or 200 bp, or more, or any number of base pairs intermediate to any of the preceding values.

Allosteric OFF→ON 5′- and 3′-Inhibited Split-Terminator Switch cgRNAs (Mechanism 7)

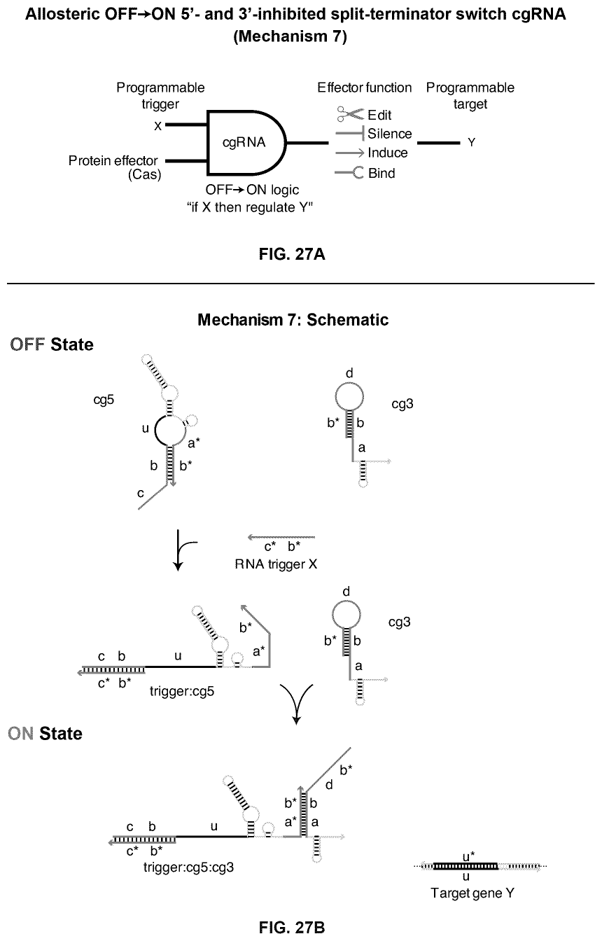

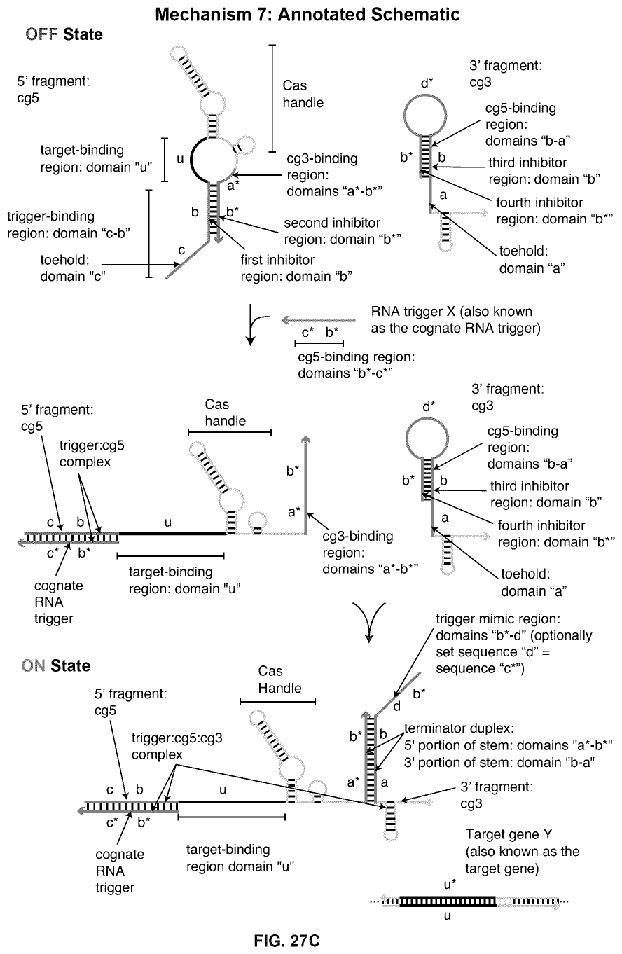

In some embodiments, allosteric OFF→ON cgRNA logic (FIG. 27A) is implemented using an allosteric OFF→ON 5′- and 3′-inhibited split-terminator switch cgRNA mechanism (Mechanism 7; FIGS. 27B and 27C). In some embodiments, the OFF→ON 5′- and 3′-inhibited split-terminator switch cgRNA (Mechanism 7; FIGS. 27B and 27C) comprises two inactive cgRNA fragments (cg5 and cg3) that are conditionally activated by RNA trigger X (the cognate RNA trigger), which binds to cg5 to allow for formation of the trigger:cg5:cg3 complex that activates the cgRNA, mediating the function of a Cas protein effector on a target gene (Y). In some embodiments, the OFF→ON 5′- and 3′-inhibited split-terminator switch cgRNA (Mechanism 7) comprises two fragments (FIGS. 27B and 27C), a 5′-fragment (cg5) and a 3′-fragment (cg3), that are inactive when not bound to each other (OFF state). In some embodiments, cg5 comprises a Cas handle, a target-binding region (sequence domain “u”) 5′ of the Cas handle, and a cg3-binding region (sequence domains “a*-b*”) 3′ of the Cas handle. In some embodiments, cg3 comprises a cg5-binding region (sequence domains “b-a”). In some embodiments, cg5 further comprises: 1) a trigger-binding region (sequence domains “c-b”) comprising a first inhibitor region (sequence domain “b” in cg5), 2) and a second inhibitor region (sequence domain “b*” in cg5); and cg3 further comprises a third inhibitor region (sequence domain “b” in cg3) and a fourth inhibitor region (sequence domain “b*” in cg3); such that in the absence of the cognate RNA trigger, the first inhibitor region (sequence domain “b” in cg5) is configured to bind to the second inhibitor region (sequence domain “b*” in cg5) and the third inhibitor region (sequence domain “b” in cg3) is configured to bind to the fourth inhibitor region (sequence domain “b*” in cg3), inhibiting binding between cg5 and cg3 by sequestering the cg3-binding region of cg5 (sequence domains “a*-b*”) in a loop and duplex (“a*” is sequestered in a loop and “b*” is sequestered in a duplex; FIGS. 27B and 27C) and by sequestering a portion of the cg5-binding region of cg3 (sequence domain “b”) in a duplex. In some embodiments, the cognate RNA trigger comprises a cg5-binding region (sequence domains “b*-c*”), the trigger-binding region of cg5 further comprises a toehold of one or more unpaired nucleotides at one or both ends (for example, domain “c”), the cg3-binding region of cg5 comprises the 5′ portion of the stem of a terminator duplex (sequence domains “a*-b*”), and the cg5-binding region of cg3 comprises the 3′ portion of the stem of the terminator duplex (sequence domain “b-a”), such that upon hybridization of the cognate RNA trigger to cg5, cg5 hybridizes to cg3 to form the terminator duplex, activating the cgRNA (ON state). In some embodiments, the target-binding region (sequence domain “u”) is non-overlapping with the trigger-binding region (sequence domains “c-b”) and is configured not to bind to the trigger-binding region, allowing for allosteric signal transduction, in which the sequence of the cognate RNA trigger (X) can be selected independently of the sequence of the target gene (Y). In some embodiments, the cognate RNA trigger comprises additional flanking nucleotides 5′ and/or 3′ of the cg5-binding region. In some embodiments, to toggle the cgRNA from the OFF state to the ON state, the RNA trigger X (cognate RNA trigger) opens up the sequestering loop and duplex in cg5 via toehold-mediated strand displacement in which the trigger first nucleates with cg5 by hybridizing to the exposed toehold “c” on cg5 and then hybridizes to domain “b” to open the loop containing domain “a*” and the duplex containing domain “b*”, facilitating hybridization between domains “a*-b*” in cg5 and domains “b-a” in cg3 to form the terminator duplex and activate the cgRNA, allowing for mediation of Cas function on the target gene (FIG. 27C). In some embodiments, once the cognate RNA trigger binds to cg5 to form complex trigger:cg5 and expose domains “a*-b*”, cg5 opens up the sequestering duplex in cg3 via toehold-mediated strand displacement in which cg5 first nucleates with cg3 by hybridizing to the exposed toehold “a” on cg3 and then hybridizes to domain “b” to open the duplex containing domain “b*”. In some embodiments, the mechanism is allosteric because the trigger and cg5 interact via domains “c-b” in cg5 and “b*-c*” in the trigger (see FIGS. 27B and 27C) that are independent of the target-binding region (domain “u” in FIGS. 27B and 27C). The resulting full sequence independence between RNA trigger X (the cognate RNA trigger) and target gene Y (the target gene) provides the flexibility for X to control regulatory scope (also known as the scope of activity) independent of the choice of Y. In some embodiments, the terminator duplex comprises, consists, or consists essentially of 2 bp, or 4 bp, 6 bp, or 8 bp, or 10 bp, or 20 bp, or 30 bp, or 40 bp, or 50 bp, or 100 bp, or 200 bp, or more, or any number of base pairs intermediate to any of the preceding values.

In some embodiments (Mechanism 7; FIGS. 27B and 27C), cg3 further comprises a trigger mimic region (sequence domains “b*-d”) with the same sequence as the cg5-binding region of the cognate RNA trigger (sequence domains “b*-c*”) (that is, in some embodiments, the sequence of domain “d” is specified to be the same as the sequence of domain “c*”), such that upon activation of the cgRNA by the cognate RNA trigger, the trigger mimic region (sequence domains “b*-d” with the sequence of domain “d” defined to be the same as the sequence of domain “c*”) is exposed. In some embodiments, this exposed trigger mimic region is then capable of serving as the cognate RNA trigger for a new copy of the cgRNA comprising a new copy of the 5′ fragment cg5 and a new copy of the 3′ fragment cg3. In some embodiments, the original cognate RNA trigger molecule is capable of triggering the ON state of a first cgRNA, which in turn is capable of trigger the ON state of a second cgRNA, which in turn is capable of trigger the ON state of a third cgRNA, and so on; this situation is equivalent to catalytic activation of multiple cgRNAs by a single cognate RNA trigger molecule.

Allosteric OFF→ON 5′- and 3′-Inhibited Split-Terminator Switch cgRNAs (Mechanism 8)

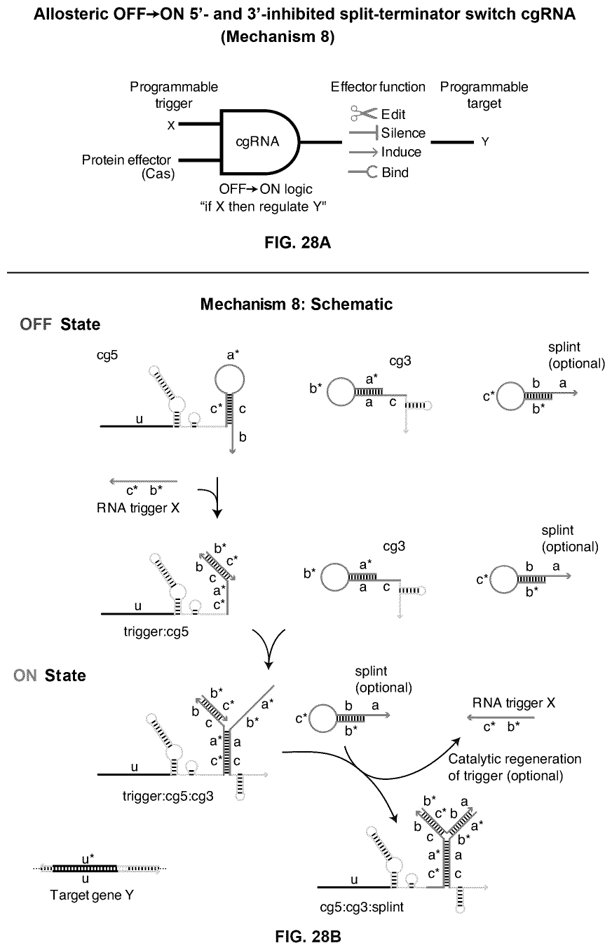

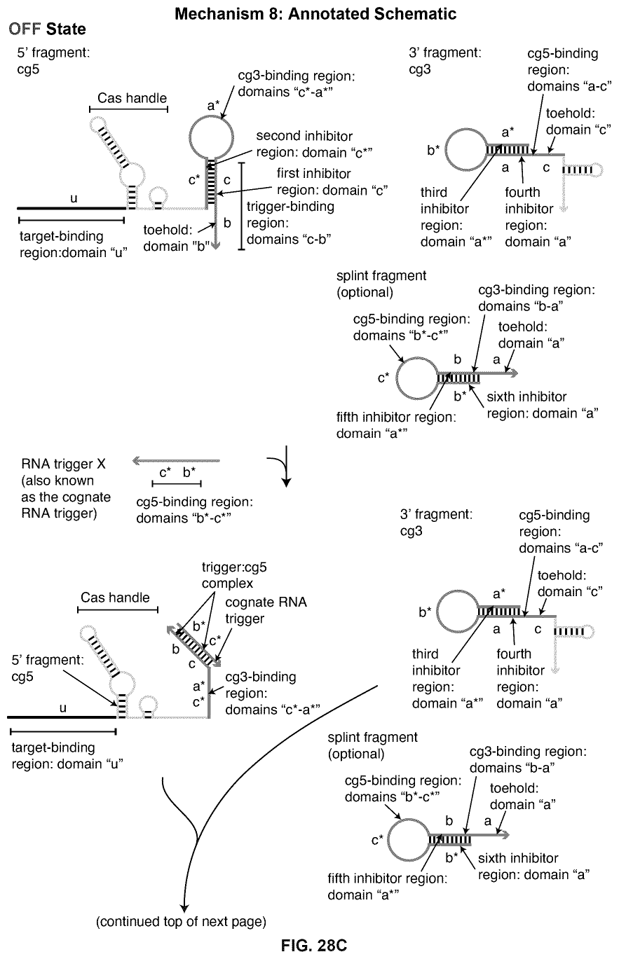

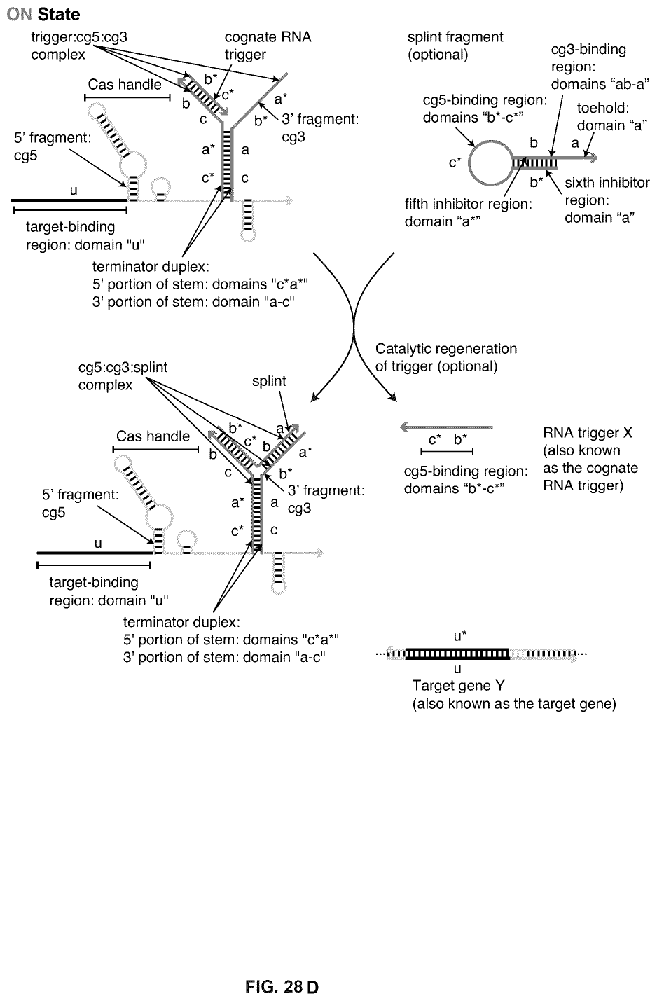

In some embodiments, allosteric OFF→ON cgRNA logic (FIG. 28A) is implemented using an allosteric OFF→ON 5′- and 3′-inhibited split-terminator switch cgRNA mechanism (Mechanism 8; FIGS. 28B and 28C). In some embodiments, the OFF→ON 5′- and 3′-inhibited split-terminator switch cgRNA (Mechanism 8; FIGS. 28B and 28C) comprises two inactive cgRNA fragments (cg5 and cg3) that are conditionally activated by RNA trigger X (the cognate RNA trigger), which binds to cg5 to allow for formation of the trigger:cg5:cg3 complex that activates the cgRNA, mediating the function of a Cas protein effector on a target gene (Y). In some embodiments, the OFF→ON 5′- and 3′-inhibited split-terminator switch cgRNA (Mechanism 8) comprises two fragments (FIGS. 28B and 28C), a 5′-fragment (cg5) and a 3′-fragment (cg3), that are inactive when not bound to each other (OFF state). In some embodiments, cg5 comprises a Cas handle, a target-binding region (sequence domain “u”) 5′ of the Cas handle, and a cg3-binding region (sequence domains “c*-a*”) 3′ of the Cas handle. In some embodiments, cg3 comprises a cg5-binding region (sequence domains “a-c”). In some embodiments, cg5 further comprises: 1) a trigger-binding region (sequence domains “c-b”) comprising a first inhibitor region (sequence domain “c” in cg5), 2) and a second inhibitor region (sequence domain “c*” in cg5); and cg3 further comprises a third inhibitor region (sequence domain “a*” in cg3) and a fourth inhibitor region (sequence domain “a” in cg3); such that in the absence of the cognate RNA trigger, the first inhibitor region (sequence domain “c” in cg5) is configured to bind to the second inhibitor region (sequence domain “c*” in cg5) and the third inhibitor region (sequence domain “a*” in cg3) is configured to bind to the fourth inhibitor region (sequence domain “a” in cg3), inhibiting binding between cg5 and cg3 by sequestering the cg3-binding region of cg5 (sequence domains “c*-a*”) in a loop and duplex (“a*” is sequestered in a loop and “c*” is sequestered in a duplex; FIGS. 28B and 28C) and by sequestering a portion of the cg5-binding region of cg3 in a duplex (the sequestered portion corresponds to domain “a” which is base paired to domain “a*” in a duplex). In some embodiments, the cognate RNA trigger comprises a cg5-binding region (sequence domains “b*-c*”), the trigger-binding region of cg5 further comprises a toehold of one or more unpaired nucleotides at one or both ends (for example, domain “b”), the cg3-binding region of cg5 comprises the 5′ portion of the stem of a terminator duplex (sequence domains “c*-a*”), and the cg5-binding region of cg3 comprises the 3′ portion of the stem of the terminator duplex (sequence domain “a-c”), such that upon hybridization of the cognate RNA trigger to cg5, cg5 hybridizes to cg3 to form the terminator duplex, activating the cgRNA (ON state). In some embodiments, the target-binding region (sequence domain “u”) is non-overlapping with the trigger-binding region (sequence domains “c-b”) and is configured not to bind to the trigger-binding region, allowing for allosteric signal transduction, in which the sequence of the cognate RNA trigger (X) can be selected independently of the sequence of the target gene (Y). In some embodiments, the cognate RNA trigger comprises additional flanking nucleotides 5′ and/or 3′ of the cg5-binding region. In some embodiments, to toggle the cgRNA from the OFF state to the ON state, the RNA trigger X (cognate RNA trigger) opens up the sequestering loop and duplex in cg5 via toehold-mediated strand displacement in which the trigger first nucleates with cg5 by hybridizing to the exposed toehold “b” on cg5 and then hybridizes to domain “c” to open the loop containing domain “a*” and the duplex containing domain “c*”, facilitating hybridization between domains “c*-a*” in cg5 and domains “a-c” in cg3 to form the terminator duplex and activate the cgRNA, allowing for mediation of Cas function on the target gene (FIG. 28C). In some embodiments, once the cognate RNA trigger binds to cg5 to form complex trigger:cg5 and expose domains “c*-a*”, cg5 opens up the sequestering duplex in cg3 via toehold-mediated strand displacement in which cg5 first nucleates with cg3 by hybridizing to the exposed toehold “c” on cg3 and then hybridizes to domain “a” to open the duplex containing domain “a*”. In some embodiments, the mechanism is allosteric because the trigger and cg5 interact via domains “c-b” in cg5 and “b*-c*” in the trigger (see FIGS. 28B and 28C) that are independent of the target-binding region (domain “u” in FIGS. 28B and 28C). The resulting full sequence independence between RNA trigger X (the cognate RNA trigger) and target gene Y (the target gene) provides the flexibility for X to control regulatory scope (also known as the scope of activity) independent of the choice of Y. In some embodiments, the terminator duplex comprises, consists, or consists essentially of 2 bp, or 4 bp, 6 bp, or 8 bp, or 10 bp, or 20 bp, or 30 bp, or 40 bp, or 50 bp, or 100 bp, or 200 bp, or more, or any number of base pairs intermediate to any of the preceding values.

In some embodiments (Mechanism 8; FIGS. 28B and 28C), cg3 additionally comprises a third fragment (known as the splint fragment) comprising a cg3-binding region (sequence domains “b-a”) comprising a fifth inhibitor region (sequence domain “b”) and a cg5-binding region (sequence domains “b*-c”) comprising a sixth inhibitor region (sequence domains “b*”) such that in the absence of the cognate RNA trigger, the fifth inhibitor region is configured to bind the sixth inhibitor region, inhibiting binding of the splint to cg5 and cg3, and such that upon activation of the cgRNA by the cognate RNA trigger, cg3 hybridizes to the cg3-binding region of the splint (sequence domains “b-a”) and cg5 hybridizes to the cg5-binding region of the splint (sequence domains “b*-c*”), displacing the trigger from cg5, corresponding to catalytic regeneration of the trigger, which can then serve as the cognate RNA trigger for a new copy of the cgRNA comprising a new copy of the 5′ fragment cg5, a new copy of the 3′ fragment cg3, and a new copy of the splint. In some embodiments, the original cognate RNA trigger molecule is capable of triggering the ON state of a first cgRNA, which in turn is capable of trigger the ON state of a second cgRNA, which in turn is capable of trigger the ON state of a third cgRNA, and so on; this situation corresponds to catalytic activation of multiple cgRNAs by a single cognate RNA trigger molecule. In some embodiments, after the cognate RNA trigger actives the cgRNA, cg3 opens up the sequestering duplex in the splint via toehold-mediated strand displacement in which cg3 first nucleates with the splint by hybridizing to the exposed toehold “a” on the splint and then hybridizes to domain “b” to open the duplex containing domain “b*”; in some embodiments, the exposed cg5-binding region (sequence domains “b*-c*”) in the splint then hybridizes to cg5 to displace the cognate RNA trigger.

Allosteric OFF→ON 5′- and 3′-Inhibited Split-Terminator Switch cgRNAs (Mechanism 9)

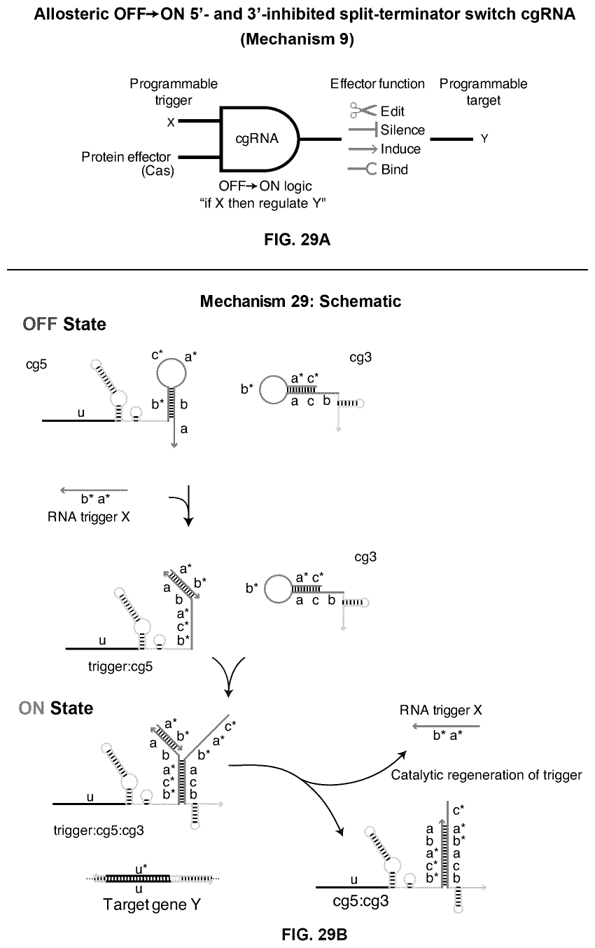

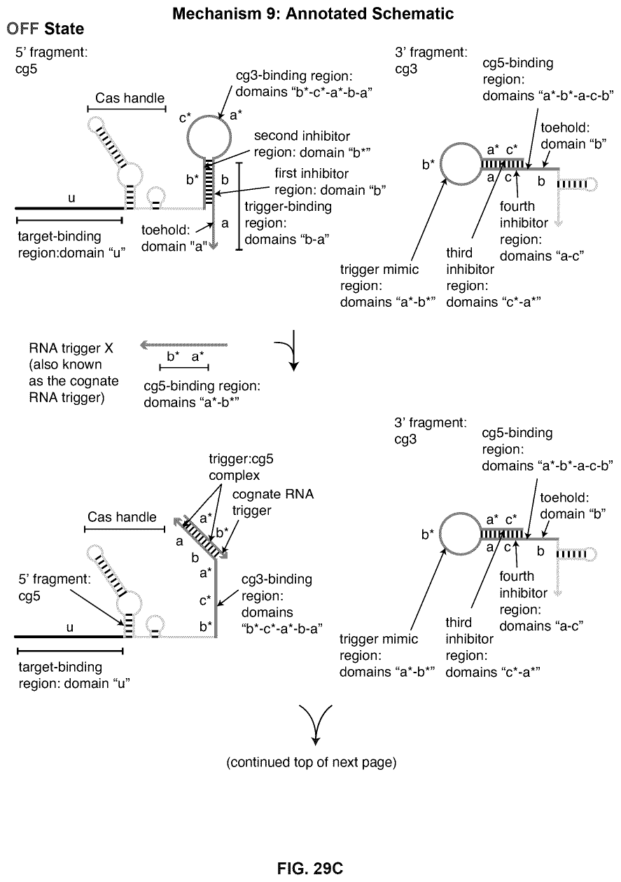

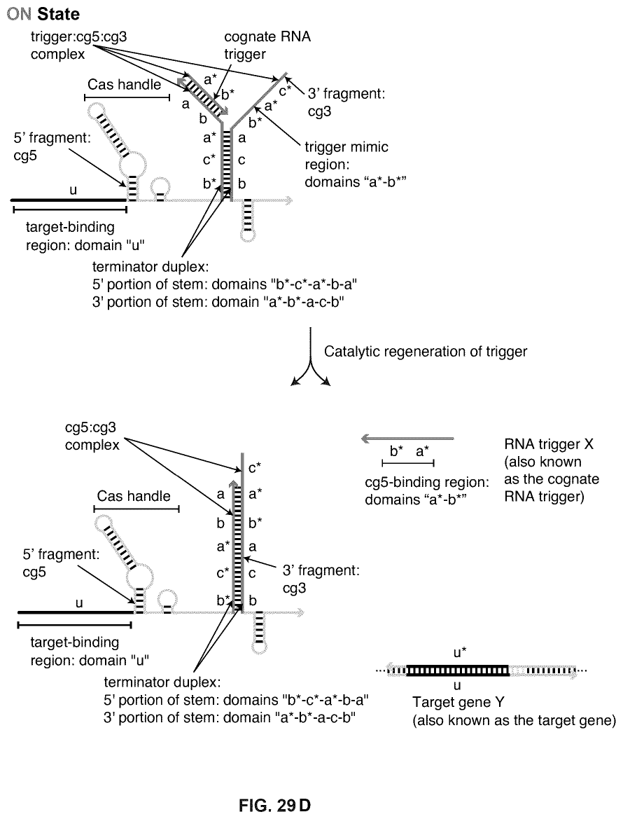

In some embodiments, allosteric OFF→ON cgRNA logic (FIG. 29A) is implemented using an allosteric OFF→ON 5′- and 3′-inhibited split-terminator switch cgRNA mechanism (Mechanism 9; FIGS. 29B and 29C). In some embodiments, the OFF→ON 5′- and 3′-inhibited split-terminator switch cgRNA (Mechanism 9; FIGS. 29B and 29C) comprises two inactive cgRNA fragments (cg5 and cg3) that are conditionally activated by RNA trigger X (the cognate RNA trigger), which binds to cg5 to allow for formation of the trigger:cg5:cg3 complex that activates the cgRNA, mediating the function of a Cas protein effector on a target gene (Y). In some embodiments, the OFF→ON 5′- and 3′-inhibited split-terminator switch cgRNA (Mechanism 9) comprises two fragments (FIGS. 29B and 29C), a 5′-fragment (cg5) and a 3′-fragment (cg3), that are inactive when not bound to each other (OFF state). In some embodiments, cg5 comprises a Cas handle, a target-binding region (sequence domain “u”) 5′ of the Cas handle, and a cg3-binding region (sequence domains “b*-c*-a*”) 3′ of the Cas handle. In some embodiments, cg3 comprises a cg5-binding region (sequence domains “a-c-b”). In some embodiments, cg5 further comprises: 1) a trigger-binding region (sequence domains “b-a”) comprising a first inhibitor region (sequence domain “b” in cg5), 2) and a second inhibitor region (sequence domain “b*” in cg5); and cg3 further comprises a third inhibitor region (sequence domains “c*-a*” in cg3) and a fourth inhibitor region (sequence domains “a-c” in cg3); such that in the absence of the cognate RNA trigger, the first inhibitor region (sequence domain “b” in cg5) is configured to bind to the second inhibitor region (sequence domain “b*” in cg5) and the third inhibitor region (sequence domains “c*-a*” in cg3) is configured to bind to the fourth inhibitor region (sequence domain “a-c” in cg3), inhibiting binding between cg5 and cg3 by sequestering the cg3-binding region of cg5 (sequence domains “b*-c*-a*”) in a loop and duplex (“c*-a*” is sequestered in a loop and “b*” is sequestered in a duplex; FIGS. 29B and 29C) and by sequestering a portion of the cg5-binding region of cg3 in a duplex (the sequestered portion corresponds to domains “a-c” which are base paired to domain “c*-a*” in a duplex). In some embodiments, the cognate RNA trigger comprises a cg5-binding region (sequence domains “a*-b*”), the trigger-binding region in cg5 further comprises a toehold of one or more unpaired nucleotides at one or both ends (for example, domain “a”), the cg3-binding region of cg5 comprises the 5′ portion of the stem of a terminator duplex (sequence domains “b*-c*-a*”), and the cg5-binding region of cg3 comprises the 3′ portion of the stem of the terminator duplex (sequence domain “a-c-b”), such that upon hybridization of the cognate RNA trigger to cg5, cg5 hybridizes to cg3 to form the terminator duplex, activating the cgRNA (ON state). In some embodiments, the target-binding region (sequence domain “u”) is non-overlapping with the trigger-binding region (sequence domains “b-a”) and is configured not to bind to the trigger-binding region, allowing for allosteric signal transduction, in which the sequence of the cognate RNA trigger (X) can be selected independently of the sequence of the target gene (Y). In some embodiments, the cognate RNA trigger comprises additional flanking nucleotides 5′ and/or 3′ of the cg5-binding region. In some embodiments, to toggle the cgRNA from the OFF state to the ON state, the RNA trigger X (cognate RNA trigger) opens up the sequestering loop and duplex in cg5 via toehold-mediated strand displacement in which the trigger first nucleates with cg5 by hybridizing to the exposed toehold “a” on cg5 and then hybridizes to domain “b” to open the loop containing domains “c*-a*” and the duplex containing domain “b*”, facilitating hybridization between domains “b*-c*-a*” in cg5 and domains “a-c-b” in cg3 to form the terminator duplex and activate the cgRNA, allowing for mediation of Cas function on the target gene (FIG. 29C). In some embodiments, once the cognate RNA trigger binds to cg5 to form complex trigger:cg5 and expose domains “b*-c*-a*”, cg5 opens up the sequestering duplex in cg3 via toehold-mediated strand displacement in which cg5 first nucleates with cg3 by hybridizing to the exposed toehold “b” on cg3 and then hybridizes to domains “c-a” to open the duplex containing domains “c*-a*”. In some embodiments, the mechanism is allosteric because the trigger and cg5 interact via domains “b-a” in cg5 and “a*-b*” in the trigger (see FIGS. 29B and 29C) that are independent of the target-binding region (domain “u” in FIGS. 29B and 29C). The resulting full sequence independence between RNA trigger X (the cognate RNA trigger) and target gene Y (the target gene) provides the flexibility for X to control regulatory scope (also known as the scope of activity) independent of the choice of Y. In some embodiments, the terminator duplex comprises, consists, or consists essentially of 2 bp, or 4 bp, 6 bp, or 8 bp, or 10 bp, or 20 bp, or 30 bp, or 40 bp, or 50 bp, or 100 bp, or 200 bp, or more, or any number of base pairs intermediate to any of the preceding values.

In some embodiments, the cg3-binding region of cg5 comprises sequence domains “b*-c*-a*-b-a” and the cg5-binding region of cg3 comprises sequence domains “a*-b*-a-c-b”. In some embodiments, the cg3-binding region of cg5 comprises the 5′ portion of the stem of a terminator duplex (sequence domains “b*-c*-a*-b-a”), and the cg5-binding region of cg3 comprises the 3′ portion of the stem of the terminator duplex (sequence domain “a*-b*-a-c-b”), such that upon hybridization of the cognate RNA trigger to cg5, cg5 hybridizes to cg3 to form the terminator duplex, activating the cgRNA (ON state). In some embodiments (Mechanism 9; FIGS. 29B and 29C), cg3 comprises a trigger mimic region (sequence domains “a*-b”) with the same sequence as the cg5-binding region of the cognate RNA trigger (sequence domains “a*-b*”) such that upon activation of the cgRNA by the cognate RNA trigger, the trigger mimic region (sequence domains “a*-b”) hybridizes to the trigger-binding region of cg5 (sequence domains “b-a”), displacing the trigger from cg5, corresponding to catalytic regeneration of the trigger which can then serve as the cognate RNA trigger for a new copy of the cgRNA comprising a new copy of the 5′ fragment cg5 and a new copy of the 3′ fragment cg3. In some embodiments, the original cognate RNA trigger molecule is capable of triggering the ON state of a first cgRNA, which in turn is capable of trigger the ON state of a second cgRNA, which in turn is capable of trigger the ON state of a third cgRNA, and so on; this situation corresponds to catalytic activation of multiple cgRNAs by a single cognate RNA trigger molecule. In some embodiments, the sequence of domain “b*” in cg3 is modified to be only partially complementary to domain “b” in cg5 (or to be non-complementary to domain “b” in cg5) so that cg3 does not displace the trigger from cg5 and the trigger is not catalytically regenerated.

### Conditional Guide RNAs

In some embodiments, the trigger comprises any of RNA, DNA, PNA, XNA, 2′OMe-RNA, amino acids, artificial amino acids, chemically modified nucleic acids, chemically modified amino acids, synthetic nucleic acid analogs, and/or chemical linkers. In some embodiments, the trigger is a combination of two or more materials. In some embodiments, the trigger molecule contains a trigger domain that is capable of toggling the activity of a cgRNA from ON→OFF or OFF→ON via a binding event. In some embodiments, the trigger molecule is an mRNA and the trigger domain is a subsequence of the mRNA. In some embodiments, the trigger molecule is an mRNA, an rRNA, a lncRNA, a miRNA, a tRNA, or any other type of endogenous or exogenous RNA comprising a subsequence that is a trigger domain capable of toggling the activity of a cgRNA (from ON→OFF or OFF→ON). In some embodiments, the trigger is a DNA molecule comprising a subsequence that is a trigger domain capable of toggling the activity of a cgRNA (from ON→OFF or OFF→ON). In some embodiments, the trigger molecule is a synthetic nucleic acid comprising a subsequence that is a trigger domain capable of toggling the activity of a cgRNA (from ON→OFF or OFF→ON) . . . . In some embodiments, the trigger molecule is expressed and comprises a subsequence that is a trigger domain capable of toggling the activity of a cgRNA (from ON→OFF or OFF→ON). In some embodiments, the trigger molecule is chemically synthesized and comprises a subsequence that is a trigger domain capable of toggling the activity of a cgRNA (from ON→OFF or OFF→ON).

In some embodiments one or more of the nucleotides in the cgRNA and/or trigger sequences is rationally designed. In some embodiments, cgRNA and/or trigger sequences are designed using the reaction pathway designer within the NUPACK the software suite,^{40,41} another piece of software, or a computational algorithm. In some embodiments, sequence design is formulated as a multistate optimization problem using a set of target test tubes to represent elementary steps in the reaction pathway as well as to model global crosstalk.⁴¹ In some embodiments, each elementary step tube contains a set of desired on-target complexes (each with a target secondary structure and target concentration), corresponding to the on-pathway hybridization products for a given step, and a set of undesired off-target complexes (each with vanishing target concentration), corresponding to on-pathway reactants and off-pathway hybridization crosstalk for a given step;⁴¹ in this scenario, these elementary step tubes promote full conversion of cognate reactants into cognate products and against local hybridization crosstalk between these same reactants. In some embodiments, to simultaneously design N orthogonal systems, elementary step tubes are specified for each orthogonal system. In some embodiments, to design against off-pathway interactions between systems, a single global crosstalk tube is also specified.⁴¹ In some embodiments, in the global crosstalk tube, the on-target complexes correspond to all reactive species generated during all elementary steps for all systems (for example, single-stranded domains). In some embodiments, in the global crosstalk tube, the off-target complexes correspond to noncognate interactions between these reactive species. In some embodiments, the global crosstalk tube ensemble omits the cognate products that the reactive species are intended to form (they appear as neither on-targets nor off-targets); in this scenario, all reactive species in the global crosstalk tube can be forced to either perform no reaction (remaining as desired on-targets) or to undergo a crosstalk reaction (forming undesired off-targets), providing the basis for minimization of global crosstalk during sequence optimization. In some embodiments, sequence design is performed subject to complementarity constraints including any combination of: 1) sequence constraints inherent to the reaction pathway (for example in FIG. 4B, domain “d” complementary to domain “d*”, etc), 2) sequence constraints imposed by the trigger sequence X, 3) sequence constraints imposed by the target Y, 4) sequence constraints imposed by the protein effector (for example, Cas, dCas, Cas9, dCas9, etc), 5) sequence constraints imposed by a synthetic terminator,⁴¹ other sequence constraints. In some embodiments, sequences are optimized by reducing the ensemble defect quantifying the average fraction of incorrectly paired nucleotides over the multi-tube ensemble.⁴¹ In some embodiments, defect weights are applied within the ensemble defect to prioritize design effort.⁴¹ In some embodiments, optimization of the ensemble defect implements both a positive design paradigm, explicitly design for on-pathway elementary steps, and a negative design paradigm, explicitly design against off-pathway crosstalk.⁴¹

In some embodiments, the cgRNA and/or trigger sequence is engineered using directed evolution. In some embodiments, the cgRNA and/or trigger sequence is engineered using a combination of rational design and directed evolution. In some embodiments, the cgRNA and/or trigger sequence is engineered using machine learning. In some embodiments, the cgRNA and/or trigger sequence is engineered using machine learning and directed evolution. In some embodiments, the cgRNA and/or trigger sequence is engineered using a combination of rational design, machine learning, and/or directed evolution.

In some embodiments, the cgRNA comprises one or more chemical modifications that alter one or more of degradation properties, affinity, biological activity, and/or delivery properties of the cgRNA, optionally wherein the one or more chemical modifications is selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′0-Methyl (20-Me) modification, and 2′0-(2-methoxyethyl) (20-MOE) modification.

In some embodiments, the trigger comprises one or more chemical modifications that alter one or more of degradation properties, affinity, biological activity, and delivery properties of the trigger, optionally wherein the one or more chemical modifications is selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′0-Methyl (20-Me) modification, and 2′0-(2-methoxyethyl) (20-MOE) modification.

In some embodiments, the sequence of the cgRNA is a subsequence of a longer RNA, DNA, or any polymer capable of base-pairing. In some embodiments, the polymer containing the cgRNA includes chemical linkers that are not capable of base-pairing. In some embodiments, the sequence of the trigger is a subsequence of a longer RNA, DNA, or any polymer capable of base-pairing. In some embodiments, the polymer containing the trigger includes chemical linkers that are not capable of base-pairing.

In some embodiments, one or more duplexes (or stems) formed by complementary or partially complementary domains of the cgRNA, 5′ fragment cg5, 3′ fragment cg3, cgRNA:inhibitor complex, cgRNA: helper complex, cg5:cg3 complex, trigger:cg5:cg3 complex, cg3: trigger complex, inhibitor: trigger complex, and/or cgRNA: trigger complex (or any of the cgRNA molecules, cgRNA fragments, triggers, inhibitors, helpers and/or complexes thereof) may contain zero, one, or more of mismatches, loops, multiloops, bulge loops, interior loops, or nicks between strands.

In some embodiments, the cgRNA, cgRNA fragments, inhibitor, and/or helper is expressed in the cells, living organisms, or artificial settings in which it interacts with effector, input, and/or target. In some embodiments, the trigger is expressed in the cells, living organisms or artificial settings in which it interacts with effector, input, and/or target. In some embodiments, the cgRNA, inhibitor, and/or helper is artificially synthesized or exogenously expressed and delivered to the cells, living organisms or artificial settings in which it interacts with effector, input, and/or target. In some embodiments, the trigger is artificially synthesized or exogenously expressed and delivered to the cells, living organisms or artificial settings in which it interacts with effector, input, and/or target. In some embodiments, the effector is expressed in the cells, living organisms or artificial settings in which it interacts with cgRNA, input, and/or target. In some embodiments, the effector is artificially synthesized or exogenously expressed and delivered to the cells, living organisms or artificial settings in which it interacts with effector, input, and/or target. In some embodiments, any of the cgRNA, cgRNA fragments, inhibitor, helper, and/or effector are delivered as a plasmid and expressed. In some embodiments, any of the cgRNA, cgRNA fragments, inhibitor, helper, and/or effector are delivered by a virus, by electroporation, by transfection, by lateral gene transfer, by microinjection, or by nanoparticle delivery. In some embodiments, any of the cgRNA, cgRNA fragments, inhibitor, helper, and/or effector are delivered as DNA and expressed.

In some embodiments, one or more of the cgRNA, cgRNA fragments, inhibitor, and/or helper strands is expressed with a self-cleaving ribozyme at one or both ends that cleaves the transcript to create the cgRNA, cgRNA fragments, inhibitor, and/or helper strands.

In some embodiments, one or more of the cgRNA, cgRNA fragments, inhibitor, and/or helper strands contains a protective element (PEL) at one or both ends, or internally, to inhibit degradation of the strand by ribonucleases.

In some embodiments, the target nucleic acid is endogenous RNA, DNA, or another polymer capable of base-pairing, whether the polymer is coding or non-coding. In some embodiments, the target nucleic acid is exogenous RNA, DNA, or another polymer capable of base-pairing, whether the polymer is coding or non-coding.

In some embodiments, the RNA guided effector is selected from the group consisting of Cas9, nickase Cas9, dCas9, silencing dCas9, inducing dCas9, catalytically dead dCas9, Cas12a, Cas13d, protein fusions or derivatives thereof, RNA-guided CRISPR effector protein or protein complex, or any protein from a similar pathway.

In some embodiments, the cgRNA conditionally performs a downstream function on a target nucleic acid in a living organism, ecosystem, tissue extract, cell lysate, and/or artificial system of reconstituted biological components. In some embodiments, the downstream effector function is the downregulation of the expression of a target nucleic acid. In some embodiments, the downstream effector function is the upregulation of the expression of a target nucleic acid. In some embodiments, the downstream effector function is the editing of the sequence of a target nucleic acid without template. In some embodiments, the downstream effector function is the editing of the sequence of a target nucleic acid with template. In some embodiments, the expression of a therapeutic target is conditionally downregulated in diseased cells or tissues. In some embodiments, the expression of a therapeutic target is conditionally upregulated in diseased cells or tissues. In some embodiments, the sequence of a therapeutic target is conditionally edited in diseased cells or tissues. In some embodiments, the regulation and/or editing of a target gene mediated by a cgRNA results in the conditional death of diseased cells or tissues. In some embodiments, the expression of a target gene is conditionally downregulated in selected prokaryotes. In some embodiments, the expression of a target gene is conditionally upregulated in selected prokaryotes. In some embodiments, the sequence of a target gene is conditionally edited in selected prokaryotes. In some embodiments, the regulation and/or editing of a target gene mediated by a cgRNA results in the conditional death of selected prokaryotes. In some embodiments, the expression of a target gene is conditionally downregulated in selected cells within a plant. In some embodiments, the expression of a target gene is conditionally upregulated in selected sells within a plant. In some embodiments, the sequence of a target gene is conditionally edited in selected cells within a plant. In some embodiments, the regulation and/or editing of a target gene mediated by a cgRNA results in the conditional death of selected cells within a plant. In some embodiments, the expression of a target gene is conditionally downregulated in a sample. In some embodiments, the expression of a target gene is conditionally upregulated in a sample. In some embodiments, the sequence of a target gene is conditionally edited in a sample. In some embodiments, the regulation and/or editing of a target gene mediated by a cgRNA results in conditional cell death in a sample. In some embodiments, the expression of a target gene is conditionally downregulated in selected prokaryotes on or within a human, pet, livestock animal, or crop plant. In some embodiments, the expression of a target gene is conditionally upregulated in selected prokaryotes on or within a human, pet, livestock animal, or crop plant. In some embodiments, the sequence of a target gene is conditionally edited in selected prokaryotes on or within a human, pet, livestock animal, or crop plant. In some embodiments, the regulation and/or editing of a target gene mediated by a cgRNA results in the conditional death of selected prokaryotes on or within a human, pet, livestock animal, or crop plant. In some embodiments, the expression of a target gene is conditionally downregulated in selected eukaryotic cells. In some embodiments, the expression of a target gene is conditionally upregulated in selected eukaryotic cells. In some embodiments, the sequence of a target gene is conditionally edited in selected eukaryotic cells. In some embodiments, the regulation and/or editing of a target gene mediated by a cgRNA results in the conditional death of selected eukaryiotic cells.

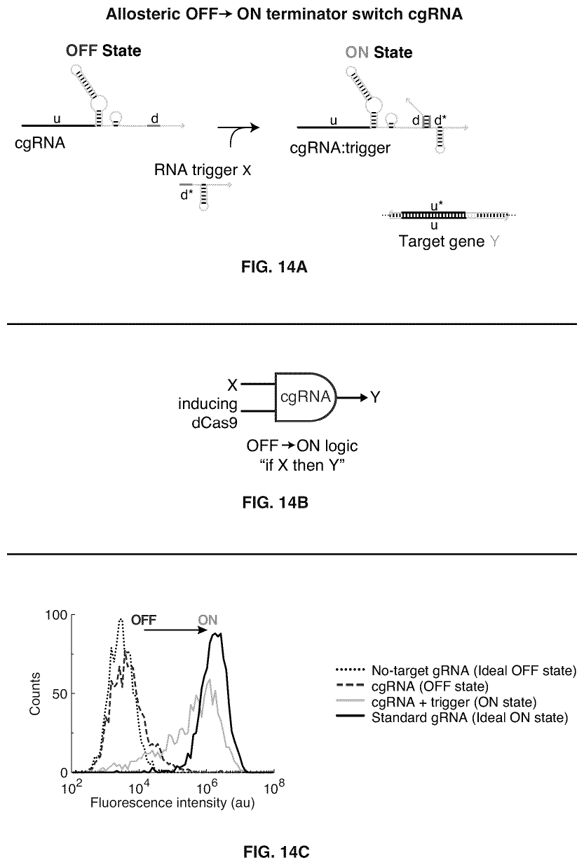

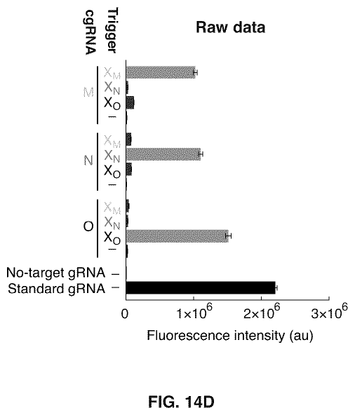

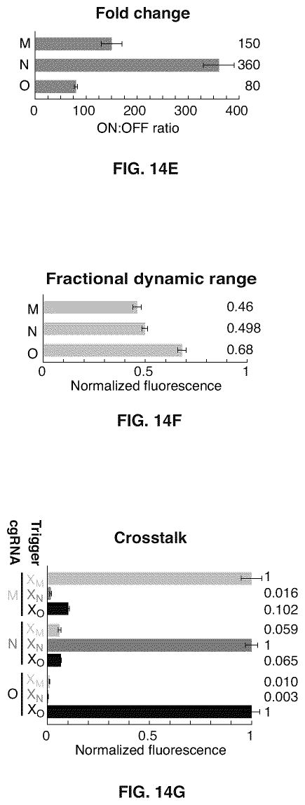

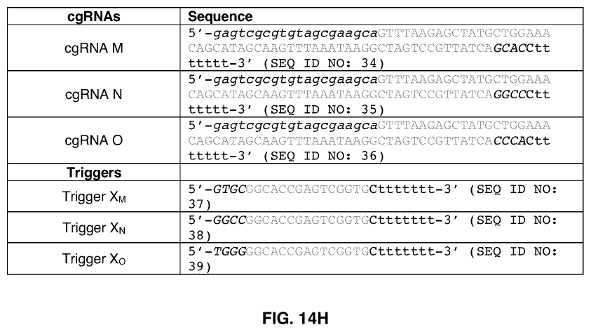

In schematics depicting allosteric cgRNAs, cgRNA fragments, cognate RNA triggers, target genes, RNA inhibitor strands, and/or RNA helper strands, and/or interactions between any of the above (for example, the schematics of FIGS. 4B, 4C, 5B, 5C, 6B, 6C, 7B, 7C, 8B, 8C, 9B, 9C, 9D, 9E, 10A, 10B, 10C, 23B, 23C, 24B, 24C, 25B, 25C, 26B, 26C, 27B, 27C, 28B, 28C, 29B, 29C), the 3′ end of each strand is depicted with an arrowhead. In some embodiments, a base-paired region in a schematic depicting allosteric cgRNAs, cognate RNA triggers, target genes, RNA inhibitor strands, and/or RNA helper strands, and/or interactions between any of the above (for example, the schematics of FIGS. 4B, 4C, 5B, 5C, 6B, 6C, 7B, 7C, 8B, 8C, 9B, 9C, 9D, 9E, 10A, 10B, 10C, 23B, 23C, 24B, 24C, 25B, 25C, 26B, 26C, 27B, 27C, 28B, 28C, 29B, 29C), comprises 1, 2, or more base pairs. In some embodiments, a base-paired region in a schematic depicting allosteric cgRNAs, cgRNA fragments, cognate RNA triggers, target genes, RNA inhibitor strands, and/or RNA helper strands, and/or interactions between any of the above (for example, the schematics of FIGS. 4B, 4C, 5B, 5C, 6B, 6C, 7B, 7C, 8B, 8C, 9B, 9C, 9D, 9E, 10A, 10B, 10C, 23B, 23C, 24B, 24C, 25B, 25C, 26B, 26C, 27B, 27C, 28B, 28C, 29B, 29C), comprises a duplex (or stem) comprising 1, 2, or more base pairs. In some embodiments, a base-paired region in a schematic depicting allosteric cgRNAs, cgRNA fragments, cognate RNA triggers, target genes, RNA inhibitor strands, and/or RNA helper strands, and/or interactions between any of the above (for example, the schematics of FIGS. 4B, 4C, 5B, 5C, 6B, 6C, 7B, 7C, 8B, 8C, 9B, 9C, 9D, 9E, 10A, 10B, 10C, 23B, 23C, 24B, 24C, 25B, 25C, 26B, 26C, 27B, 27C, 28B, 28C, 29B, 29C), comprises a duplex (or stem) comprising 1, 2, or more consecutive base pairs. In some embodiments, a base-paired region in a schematic depicting allosteric cgRNAs, cgRNA fragments, cognate RNA triggers, target genes, RNA inhibitor strands, and/or RNA helper strands, and/or interactions between any of the above (for example, the schematics of FIGS. 4B, 4C, 5B, 5C, 6B, 6C, 7B, 7C, 8B, 8C, 9B, 9C, 9D, 9E, 10A, 10B, 10C, 23B, 23C, 24B, 24C, 25B, 25C, 26B, 26C, 27B, 27C, 28B, 28C, 29B, 29C) is optional. In some embodiments, any of allosteric cgRNAs, cgRNA fragments, cognate RNA triggers, RNA inhibitor strands, and/or RNA helper strands can be extended at either or both of the 5′ or 3′ ends (or internally) by zero, one, or more additional domains, wherein any additional domains may be unstructured, may serve as a protective element (PEL) to reduce RNA degradation, may serve as a toehold, may comprise additional base-pairs, or any combination of the above. In some embodiments, the cognate RNA trigger is a subsequence of an endogenous RNA. In some embodiments, any of allosteric cgRNAs, cgRNA fragments, cognate RNA triggers, RNA inhibitor strands, and/or RNA helper strands may include one or more chemically modified nucleotides or synthetic nucleic acid analogs or other materials capable of base-pairing (including but not limited to DNA, XNA, PNA, 2′OMe-RNA) and/or one or more chemical linkers that are not capable of base-pairing. In some embodiments, the Cas9 handle is a modified handle with a different structure and/or sequence relative to a wildtype Cas9 handle (for example, the “flip+extend” (FE)-modified Cas9 handle used in the experimental studies of FIGS. 14-16) 42. In some embodiments, the structure and/or sequence of the Cas handle is modified relative to a wildtype Cas sequence via rational design, machine learning, directed evolution, or any combination thereof.

In some embodiments, additional regions or sequence domains may be added at either end of any of the depicted regions or sequence domains. In some embodiments, one or more depicted regions (e.g., sequence domains or nucleotides) are optional.

In some embodiments, an inactive cgRNA (for example, Mechanisms 1B, 2B, 3B, 4A, 5, 6, 7, 8, 9) can be activated by a first trigger sequence (OFF→ON logic). In some embodiments, a cgRNA that is activated by a first trigger sequence (OFF→ON logic) can later be inactivated by second trigger sequence (ON→OFF logic). In some embodiments, a cgRNA can be cycled between the ON and OFF states by first and second trigger sequences.

In some embodiments, an active cgRNA (for example, Mechanisms 1A, 2A, 3A, 4B, 4C) can be inactivated by a first trigger sequence (ON→OFF logic). In some embodiments, a cgRNA that is inactivated by a first trigger sequence (OFF→ON logic) can later be activated by second trigger sequence (ON→OFF logic). In some embodiments, a cgRNA can be cycled between the ON and OFF states by first and second trigger sequences.

In some embodiments, a cgRNA can catalytically regenerate the trigger sequence (for example, Mechanisms 7, 8, 9) so that one trigger molecule can toggle the state of multiple copies of the cgRNA.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, any invention herein is not intended to be limited by the recitation of the preferred embodiments, but is instead to be defined by reference to the appended claims. All references cited herein are incorporated by reference in their entirety.

### Arrangements

In addition to the foregoing, some embodiments provide the following arrangements:

Arrangement 1: An allosteric conditional guide RNA (cgRNA) comprising a target-binding region and a trigger-binding region: a, wherein the target-binding region is non-overlapping with the trigger-binding region; b, wherein the cgRNA is active in the absence of a cognate RNA trigger, wherein the cgRNA is configured to mediate the function of a Cas protein effector on a target gene that binds the target-binding region; and c, wherein upon hybridization to the cognate RNA trigger, the cgRNA is inactivated, inhibiting further mediation of Cas function on the target gene.

Arrangement 2: The allosteric cgRNA of Arrangement 1, further comprising a Cas handle wherein the target-binding region is 5′ of the Cas handle and the trigger-binding region is 3′ of the Cas handle.

Arrangement 3: The allosteric cgRNA of Arrangement 1, the cgRNA further comprising a first terminator hairpin with an extended loop comprising 5 or more nucleotides wherein the trigger-binding region comprises: a. zero, one, or more nucleotides of a linker 5′-adjacent to the first terminator hairpin; b. zero, one, or more nucleotides in a 5′ portion of a stem of the first terminator hairpin; and c. one or more nucleotides in the extended loop of the first terminator hairpin; wherein the cgRNA is inactivated upon hybridization of the cognate RNA trigger to the cgRNA.

Arrangement 4: The allosteric cgRNA of Arrangement 1, further comprising a Cas handle with an extended loop wherein the target-binding region is 5′ of the Cas handle and the trigger-binding region comprises a portion of the extended loop of the Cas handle and no nucleotides 5′ of the Cas handle.

Arrangement 5: The allosteric cgRNA of Arrangement 4, further comprising a first terminator hairpin with an extended loop comprising 5 or more nucleotides such that the trigger-binding region comprises: a. one or more nucleotides in the extended loop of the Cas handle; and b. one or more nucleotides in the extended loop of the first terminator hairpin, wherein upon hybridization of the cognate RNA trigger to the cgRNA, the cgRNA is inactivated.

Arrangement 6: The allosteric cgRNA of Arrangement 4, the cgRNA further comprising a first terminator hairpin with an extended loop comprising 5 or more nucleotides wherein the trigger-binding region further comprises: a. zero, one, or more nucleotides of a linker 5′-adjacent to the first terminator hairpin; b. zero, one, or more nucleotides in a 5′ portion of a stem of the first terminator hairpin; and c. one or more nucleotides in the extended loop of the first terminator hairpin, wherein the cgRNA is inactivated upon hybridization of the cognate RNA trigger to the cgRNA.

Arrangement 7: An allosteric conditional guide RNA (cgRNA) and an RNA inhibitor strand, wherein the cgRNA comprises a target-binding region and an inhibitor-binding region, and the RNA inhibitor strand comprises a trigger-binding region, wherein the cgRNA is configured to bind to a portion of the trigger-binding region to form a cgRNA:inhibitor complex: a, wherein the target-binding region is not base-paired to the trigger-binding region in the cgRNA:inhibitor complex; b, wherein the cgRNA:inhibitor complex is inactive in the absence of a cognate RNA trigger; and c, wherein upon hybridization of a cognate RNA trigger to the inhibitor, the cgRNA is activated, mediating the function of a Cas protein effector on a target gene that binds the target-binding region.

Arrangement 8: The allosteric cgRNA and RNA inhibitor strand of Arrangement 7, the cgRNA further comprising a Cas handle wherein the target-binding region is 5′ of the Cas handle and the inhibitor-binding region is 3′ of the Cas handle.

Arrangement 9: The allosteric cgRNA and RNA inhibitor strand of Arrangement 7, the inhibitor further comprising a toehold of one or more unpaired nucleotides at one or both ends, the cgRNA further comprising a first terminator hairpin with an extended loop comprising 5 or more nucleotides, the cgRNA further comprising an inhibitor-binding region comprising: a. zero, one, or more nucleotides of a linker 5′-adjacent to the first terminator hairpin; b. zero, one, or more nucleotides in a 5′ portion of a stem of the first terminator hairpin; and c. one or more nucleotides in the extended loop of the first terminator hairpin, wherein the cgRNA is inactive in the cgRNA:inhibitor complex, and wherein hybridization of the cognate RNA trigger to the inhibitor displaces the cgRNA from the inhibitor, thereby activating the cgRNA.

Arrangement 10: The allosteric cgRNA and RNA inhibitor strand of Arrangement 7, the cgRNA further comprising a Cas handle with an extended loop wherein the target-binding region is 5′ of the Cas handle, and wherein the inhibitor-binding region comprises a portion of the extended loop of the Cas handle and no nucleotides 5′ of the Cas handle.

Arrangement 11: The allosteric cgRNA and RNA inhibitor strand of Arrangement 10, the inhibitor further comprising a toehold of one or more unpaired nucleotides at one or both ends, the cgRNA further comprising a first terminator hairpin with an extended loop comprising 5 or more nucleotides, and the inhibitor-binding region comprising: a. one or more nucleotides in the extended loop of the Cas handle; and b. one or more nucleotides in the extended loop of the first terminator hairpin; wherein the cgRNA is inactive in the cgRNA:inhibitor complex, and wherein hybridization of the cognate RNA trigger to the inhibitor displaces the cgRNA from the inhibitor, thereby activating the cgRNA.

Arrangement 12: The allosteric cgRNA and RNA inhibitor strand of Arrangement 10, the inhibitor further comprising a toehold of one or more unpaired nucleotides at one or both ends, the cgRNA further comprising a first terminator hairpin with an extended loop comprising 5 or more nucleotides, and the inhibitor-binding region comprising: a. one or more nucleotides in the extended loop of the Cas handle; b. zero, one, or more nucleotides of a linker 5′-adjacent to the first terminator hairpin; c. zero, one, or more nucleotides of a 5′ portion of a stem of the first terminator hairpin; and d. one or more nucleotides in the extended loop of the first terminator hairpin; wherein the cgRNA is inactive in the cgRNA:inhibitor complex, and wherein hybridization of the cognate trigger to the inhibitor displaces the cgRNA from the inhibitor, thereby activating the cgRNA.

Arrangement 13: An allosteric conditional guide RNA (cgRNA) comprising a 5′ fragment (cg5) and a 3′ fragment (cg3), cg5 comprising a target-binding region and a trigger-binding region, and cg3 comprising a cognate RNA trigger: a, wherein the target-binding region is non-overlapping with the trigger-binding region; b, wherein cg5 and cg3 are inactive when not bound to each other; and c, wherein upon hybridization of cg3 to cg5 to form a cg5:cg3 complex, the cgRNA is activated, mediating the function of a Cas protein effector on a target gene that binds the target-binding region.

Arrangement 14: The allosteric cgRNA of Arrangement 13 wherein the fragment cg5 further comprises a Cas handle wherein the target-binding region is 5′ of the Cas handle and the trigger-binding region is 3′ of the Cas handle.

Arrangement 15: The allosteric cgRNA of Arrangement 13, wherein fragment cg5 further comprises a trigger-binding region comprising a 5′ portion of a stem of a terminator duplex; and fragment cg3 further comprises a 3′ portion of the stem of the terminator duplex, such that hybridization of cg5 to cg3 forms the terminator duplex, activating the cgRNA.

Arrangement 16: An allosteric conditional guide RNA (cgRNA) comprising a 5′ fragment (cg5) and a 3′ fragment (cg3), cg5 comprising a target-binding region and a trigger-binding region, and cg3 configured to bind to a portion of the trigger-binding region to form a cg5:cg3 complex: a, wherein the target-binding region is non-overlapping with the trigger-binding region; b, wherein the cg5:cg3 complex is active in the absence of a cognate RNA trigger, mediating the function of a Cas protein effector on a target gene that binds the target-binding region; c, wherein cg5 and cg3 are inactive when not bound to each other; and d, wherein hybridization of the cognate RNA trigger to cg5 displaces cg3 from cg5, thereby inhibiting further mediation of Cas function on the target gene.

Arrangement 17: The allosteric cgRNA of Arrangement 16, wherein the fragment cg5 further comprises a Cas handle wherein the target-binding region is 5′ of the Cas handle and the trigger-binding region is 3′ of the Cas handle.

Arrangement 18: The allosteric cgRNA of Arrangement 16, wherein the fragment cg5 further comprises a trigger-binding region comprising: a. a 5′ portion of a stem of a terminator duplex; b. zero, one, or more nucleotides of a linker 5′-adjacent to the 5′ portion of the stem of the terminator duplex; and c. a toehold comprising zero, one, or more nucleotides 3′-adjacent to the 5′ portion of the stem of the terminator duplex; wherein the fragment cg3 further comprises a 3′ portion of the stem of the terminator duplex, wherein hybridization of cg5 to cg3 forms the terminator duplex within the cg5:cg3 complex, and wherein hybridization of the trigger to cg5 displaces cg3 from cg5, thereby breaking the terminator duplex and inactivating the cgRNA.

Arrangement 19: An allosteric conditional guide RNA (cgRNA) comprising: a 5′ fragment (cg5) and a 3′ fragment (cg3), cg5 comprising a target-binding region; and cg3 comprising a trigger-binding region, wherein cg5 is configured to bind to a portion of the trigger-binding region to form a cg5:cg3 complex: a, wherein the target-binding region is not base-paired to the trigger-binding region in the cg5:cg3 complex; b, wherein the cg5:cg3 complex is active in the absence of a cognate RNA trigger, mediating the function of a Cas protein effector on a target gene that binds the target-binding region; c, wherein cg5 and cg3 are inactive when not bound to each other; and d, wherein hybridization of the cognate RNA trigger to cg3 displaces cg5 from cg3, thereby inhibiting further mediation of Cas function on the target gene.

Arrangement 20: The allosteric cgRNA of Arrangement 19, wherein the fragment cg5 further comprises a Cas handle wherein the target-binding region is 5′ of the Cas handle, and wherein the fragment cg3 binds to cg5 3′ of the Cas handle.

Arrangement 21: The allosteric cgRNA of Arrangement 19, wherein: the fragment cg3 further comprises a trigger-binding region comprising: a. a 3′ portion of a stem of a terminator duplex, and b. a toehold comprising zero, one, or more nucleotides 5′-adjacent to the 3′ portion of the stem of the terminator duplex; the fragment cg5 further comprises a 5′ portion of the stem of the terminator duplex; and wherein hybridization of cg5 to cg3 forms the terminator duplex within the cg5:cg3 complex, and wherein hybridization of the cognate RNA trigger to cg3 displaces cg5 from cg3, thereby breaking the terminator duplex and inactivating the cgRNA.

Arrangement 22: An allosteric conditional guide RNA (cgRNA) comprising: a 5′ fragment (cg5) and a 3′ fragment (cg3), wherein cg5 comprises a Cas handle, a target-binding region 5′ of the Cas handle, and a cg3-binding region 3′ of the Cas handle, wherein cg3 comprises a cg5-binding region, and wherein either cg5 or cg3 comprises a trigger-binding region: a, wherein the target-binding region is non-overlapping with the trigger-binding region and is configured not to bind to the trigger-binding region; b, wherein cg5 and cg3 are inactive when not bound to each other; c, wherein in the absence of a cognate RNA trigger, cg5 and cg3 are configured to be inhibited from binding to each other; and d, wherein upon hybridization of the cognate RNA trigger to either cg5 or cg3, cg5 and cg3 hybridize to form a trigger:cg5:cg3 complex that activates the cgRNA, thereby mediating the function of a Cas protein effector on a target gene that binds the target-binding region.

Arrangement 23: The allosteric cgRNA of Arrangement 22, wherein the fragment cg5 further comprises: a. a trigger-binding region comprising a first inhibitor region, and b. a second inhibitor region; wherein in the absence of the cognate RNA trigger, the first inhibitor region is configured to bind to the second inhibitor region, thereby inhibiting binding between cg5 and cg3.

Arrangement 24: The allosteric cgRNA of Arrangement 23, wherein: a. the cognate RNA trigger comprises a cg5-binding region; b. the trigger-binding region further comprises a toehold of one or more unpaired nucleotides at one or both ends; c. the cg3-binding region of cg5 comprises a 5′ portion of a stem of a terminator duplex; and d. the cg5-binding region of cg3 comprises a 3′ portion of the stem of the terminator duplex; wherein upon hybridization of the cognate RNA trigger to cg5, cg5 hybridizes to cg3 to form the terminator duplex, thereby activating the cgRNA.

Arrangement 25: The allosteric cgRNA of Arrangement 22, wherein cg3 further comprises: a. a trigger-binding region comprising a first inhibitor region, and b. a second inhibitor region; wherein in the absence of the cognate RNA trigger, the first inhibitor region is configured to bind the second inhibitor region, thereby inhibiting binding between cg5 and cg3.

Arrangement 26: The allosteric cgRNA of Arrangement 25, wherein: a. the cognate RNA trigger comprises a cg3-binding region; b. the trigger-binding region further comprises a toehold of one or more unpaired nucleotides at one or both ends; c. the cg5-binding region of cg3 comprises a 3′ portion of a stem of a terminator duplex; and d. the cg3-binding region of cg5 comprises a 5′ portion of the stem of the terminator duplex; wherein upon hybridization of the cognate RNA trigger to cg3, cg3 hybridizes to cg5 to form the terminator duplex, thereby activating the cgRNA.

Arrangement 27: The allosteric cgRNA of Arrangement 22 wherein cg5 further comprises: a. a trigger-binding region comprising a first inhibitor region; and b. a second inhibitor region; and wherein cg3 further comprises: c. a third inhibitor region; and d. a fourth inhibitor region, wherein in the absence of a cognate RNA trigger, the first inhibitor region is configured to bind the second inhibitor region and the third inhibitor region is configured to bind to the fourth inhibitor region, thereby inhibiting binding between cg5 and cg3.

Arrangement 28: The allosteric cgRNA of Arrangement 27, wherein: a. the cognate RNA trigger comprises a cg5-binding region; b. the trigger-binding region of cg5 further comprises a toehold of one or more unpaired nucleotides at one or both ends; c. the cg3-binding region of cg5 comprises a 5′ portion of a stem of a terminator duplex; and d. the cg5-binding region of cg3 comprises a 3′ portion of the stem of the terminator duplex comprising a toehold of one or more unpaired nucleotides at one or both ends; wherein upon hybridization of the cognate RNA trigger to cg5, cg5 hybridizes to cg3 to form the terminator duplex, thereby activating the cgRNA.

Arrangement 29: The allosteric cgRNA of Arrangement 27 wherein the trigger-binding region of cg5 is 5′ of the target-binding region.

Arrangement 30: The allosteric cgRNA of any one of Arrangements 27, 28, and 29, wherein cg3 comprises a trigger mimic region having a sequence identical to that of the cg5-binding region of the cognate RNA trigger, wherein upon activation of the cgRNA by the cognate RNA trigger, the trigger mimic region is exposed and capable of serving as the cognate RNA trigger for a new copy of the cgRNA comprising a new copy of the 5′ fragment cg5 and a new copy of the 3′ fragment cg3.

Arrangement 31: The allosteric cgRNA of Arrangement 27 wherein the trigger-binding region of cg5 is 3′ of the Cas handle.

Arrangement 32: The allosteric cgRNA of any one of Arrangements 27, 28, and 31 wherein the cgRNA additionally comprises a splint as a third fragment wherein the splint comprises: a. a cg3-binding region comprising a fifth inhibitor region and further comprising a toehold of one or more unpaired nucleotides at one or both ends; and b. a cg5-binding region comprising a sixth inhibitor region; wherein in the absence of a cognate RNA trigger, the fifth inhibitor region is configured to bind the sixth inhibitor region, inhibiting binding of the splint to cg5 and cg3, and wherein upon activation of the cgRNA by the cognate RNA trigger, cg3 hybridizes to the cg3-binding region of the splint and the cg5-binding region of the splint hybridizes to cg5, displacing the trigger from cg5, and catalytically regenerating the trigger which then serves as the cognate RNA trigger for a new copy of the cgRNA comprising a new copy of the 5′ fragment cg5 and a new copy of the 3′ fragment cg3.

Arrangement 33: The allosteric cgRNA of Arrangement 32, wherein the catalytically regenerated trigger serves as the cognate RNA trigger for a new copy of the cgRNA that further comprises a new copy of the splint fragment.

Arrangement 34: The allosteric cgRNA of any one of Arrangements 27, 28, and 31, wherein cg3 comprises a trigger mimic region having a sequence identical to that of the cg5-binding region of the cognate RNA trigger, wherein upon activation of the cgRNA by the cognate RNA trigger, the trigger mimic region hybridizes to the trigger-binding region of cg5, displacing the trigger from cg5, and catalytically regenerating the trigger which can then serve as the cognate RNA trigger for a new copy of the cgRNA comprising a new copy of the 5′ fragment cg5 and a new copy of the 3′ fragment cg3.

Arrangement 35: The allosteric cgRNA of any one of Arrangements 1 to 34 where the trigger is an RNA.

Arrangement 36: The allosteric cgRNA of any one of Arrangements 1 to 34 where the trigger is or is a subsequence of an mRNA, an rRNA, a lncRNA, a miRNA, or a tRNA.

Arrangement 37: The allosteric cgRNA of any one of Arrangements 1 to 34 where the cgRNA is expressed in a cell.

Arrangement 38: The allosteric cgRNA of any one of Arrangements 1 to 34 where the cgRNA is chemically synthesized.

Arrangement 39: The allosteric cgRNA of any one of the preceding Arrangements, wherein the cgRNA, cgRNA fragment, RNA inhibitor strand, and/or trigger further comprises one or more additional regions at the 5′ and/or the 3′ end.

Arrangement 40: The allosteric cgRNA of any one of the preceding Arrangements, wherein the cgRNA, cgRNA fragment, RNA inhibitor strand, and/or trigger comprises one or more chemical modifications that alter one or more of degradation properties, affinity, biological activity, and/or delivery properties of the cgRNA.

Arrangement 41: The allosteric cgRNA of any one of the preceding Arrangements, wherein the cgRNA, cgRNA fragment, RNA inhibitor strand, and/or trigger comprises one or more chemical modifications selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′0-Methyl (20-Me) modification, and 2′0-(2-methoxyethyl) (20-MOE) modification.

Arrangement 42: The allosteric cgRNA of any one of the preceding Arrangements, wherein an RNA trigger, RNA helper, and/or RNA inhibitor further comprises a protective element (PEL), wherein none, some, or all of the PEL sequence is derived from a component of a viral xrRNA sequence, and wherein the PEL reduces degradation of the RNA trigger, RNA helper, and/or RNA inhibitor in a prokaryotic or eukaryotic cell.

Arrangement 43: The allosteric cgRNA of any one of the preceding Arrangements, wherein the cgRNA and/or one or more cgRNA fragments further comprise a protective element (PEL), wherein none, some, or all of the PEL sequence is derived from a component of a viral xrRNA, and wherein the PEL reduces degradation of the cgRNA and/or the cgRNA fragments in a prokaryotic or eukaryotic cell.

Arrangement 44: The allosteric cgRNA of any one of the preceding Arrangements, wherein the cgRNA works in conjunction with Cas to mediate cell-selective induction, silencing, editing, or binding of a target gene.

Arrangement 45: A method of conditionally inhibiting mediation of Cas function on a target gene, comprising: providing an allosteric conditional guide RNA (cgRNA) according to any one of Arrangements 1 to 6 and 35-44; and combining the cgRNA with a system comprising a target gene and a Cas protein effector; wherein the cgRNA is active in mediating the function of the Cas protein effector on the target gene in the absence of a cognate RNA trigger, and wherein upon hybridization to the cognate RNA trigger, the cgRNA is inactivated, inhibiting further mediation of Cas function on the target gene.

Arrangement 46: A method of conditionally mediating the function of a Cas protein effector on a target gene, comprising: providing an allosteric conditional guide RNA (cgRNA) and an RNA inhibitor strand according to any one of Arrangements 7 to 12 and 35-44; and combining the cgRNA and RNA inhibitor strand with a system comprising a target gene and a Cas protein effector; wherein in the absence of a cognate RNA trigger, the inhibitor is bound to the cgRNA and the cgRNA is inactive; and wherein upon hybridization of a cognate RNA trigger to the inhibitor, the cgRNA is activated, mediating the function of a Cas protein effector on the target gene.

Arrangement 47: A method of conditionally mediating the function of a Cas protein effector on a target gene, comprising: providing an allosteric conditional guide RNA (cgRNA) comprising a 5′ fragment (cg5) and a 3′ fragment (cg3) according to any one of Arrangements 13 to 15 and 35-44; and combining the cgRNA with a system comprising a target gene and a Cas protein effector; wherein cg5 and cg3 are inactive when not bound to each other; and wherein upon hybridization of cg3 to cg5, the cgRNA is activated, mediating the function of a Cas protein effector on the target gene.

Arrangement 48: A method of conditionally inhibiting mediation of Cas function on a target gene, comprising: providing an allosteric conditional guide RNA (cgRNA) comprising a 5′ fragment (cg5) and a 3′ fragment (cg3) according to any one of Arrangements 16 to 18 and 35-44; and combining the cgRNA with a system comprising a target gene and a Cas protein effector; wherein in the absence of a cognate RNA trigger cg5 is bound to cg3 and the cgRNA is active; and wherein hybridization of the cognate RNA trigger to cg5 displaces cg3 from cg5, thereby inhibiting further mediation of Cas function on the target gene.

Arrangement 49: A method of conditionally inhibiting mediation of Cas function on a target gene, comprising: providing an allosteric conditional guide RNA (cgRNA) comprising a 5′ fragment (cg5) and a 3′ fragment (cg3) according to any one of Arrangements 19 to 21 and 35-44; and combining the cgRNA with a system comprising a target gene and a Cas protein effector; wherein in the absence of a cognate RNA trigger cg5 is bound to cg3 and the cgRNA is active; and wherein hybridization of the cognate RNA trigger to cg3 displaces cg5 from cg3, thereby inhibiting further mediation of Cas function on the target gene.

Arrangement 50: A method of conditionally mediating the function of a Cas protein effector on a target gene, comprising: providing an allosteric conditional guide RNA (cgRNA) comprising a 5′ fragment (cg5) and a 3′ fragment (cg3) according to any one of Arrangements 22 to 34 and 35-44; and combining the cgRNA with a system comprising a target gene and a Cas protein effector; wherein in the absence of a cognate RNA trigger, cg5 and cg3 are inhibited from binding to each other and the cgRNA is inactive; and wherein upon hybridization of the cognate RNA trigger to either cg5 or cg3, cg5 and cg3 hybridize to form a trigger:cg5:cg3 complex that activates the cgRNA, thereby mediating the function of a Cas protein effector on the target gene.

### EXAMPLES

### Example—Logic, Function, Structure, and Interactions of a Standard Guide RNA (gRNA)

FIG. 1A depicts the logic and function of a standard guide RNA (gRNA). A standard gRNA is always ON, unconditionally directing the activity of a protein effector to a target Y; different Cas9, dCas9, and/or Cas variants implement different functions (for example, edit, silence, induce, bind). FIG. 1B depicts structure and interactions of a standard gRNA. From 5′ to 3′, a standard gRNA comprises: a target-binding region, a Cas handle recognized by the protein effector, and a terminator region.

### Example—Logic and Function of a Conditional Guide RNA (cgRNA)

FIG. 2 depicts the logic and function of a conditional guide RNA (cgRNA) in which a cgRNA changes conformation in response to a programmable trigger X to conditionally direct the activity of a protein effector to a programmable target Y. FIG. 2A depicts ON→OFF logic with a constitutively active cgRNA that is conditionally inactivated by X. FIG. 2B depicts OFF→ON logic with a constitutively inactive cgRNA that is conditionally activated by X.

### Example—Applications of Cell-Selective Control of Regulatory Control with cgRNAs

FIG. 3 illustrates applications of cell-selective regulation of CRISPR/Cas function using cgRNAs. FIG. 3A contrasts global silencing (top arrow) of target gene Y using silencing dCas9 and a standard gRNA that implements the unconditional logic “silence Y” to cell-selective silencing (bottom arrow) of target gene Y using silencing dCas9 and a conditional cgRNA, such that Y is silenced locally only where X is expressed. FIG. 3B illustrates diverse modes of cell-selective spatiotemporal regulatory control using cgRNA conditional logic (ON→OFF or OFF→ON) and different Cas9 functional variants (induce, silence, edit, bind, etc). ON→OFF and OFF→ON cgRNAs produce inverted regulatory patterns on target Y in response to a given pattern for trigger X. FIG. 3C illustrates some cell-selective and tissue-selective tools. For example, conditional gene silencing (“if gene X is transcribed, silence independent gene Y”) can be used to probe genetic necessity, conditional gene activation (“if gene X is transcribed, activate independent gene Y”) can be used to probe genetic sufficiency, and conditional cell death (“if gene X is transcribed, induce apoptosis”) can be used to probe developmental compensation. In each case, conditional regulation is mediated by a cgRNA whose activity is toggled by a programmable trigger X. For some embodiments, by selecting a trigger X with the desired spatial and temporal expression profiles, the regulatory function is restricted to a desired cell type, tissue, or organ within an organism, mixture of cells, or ecosystem. For some embodiments, to shift conditional regulation to a different tissue type or time point, the cgRNAs can be programmed to recognize a different trigger X. For some embodiments, to enhance cell-selective spatiotemporal control in multi-cellular settings (e.g., within embryos or bacterial mixtures), multi-input conditional logic (operating on two or more inputs X₁, X₂, . . . ) using AND gates can be used to narrow the scope of regulation on Y; alternatively, OR gates can be used to broaden the scope of regulation on Y. In some embodiments, AND logic is implemented using split-cgRNAs that are functional only in the presence of both X₁ and X₂. In some embodiments, OR logic is executed using multiple cgRNA variants that accept different inputs (X₁, X₂, . . . ) but target the same output Y. FIG. 3D illustrates one example of cgRNA-mediated cell-selective reporter regulation for multiplexed in vivo RNA imaging. Four cgRNAs each detect a different mRNA input (mRNA₁, mRNA₂, mRNA₃, mRNA₄) that serves as an RNA trigger, activating the corresponding cgRNA to induce the corresponding spectrally distinct FP reporter (FP₁, FP₂, FP₃, FP₄). After once optimizing a plasmid-based reporter system expressing inducing dCas9, the 4 FP reporters, and the 4 cgRNAs, imaging a new set of mRNAs requires only updating the sequences of the cgRNAs to accept new mRNAs as triggers. This cgRNA approach offers important conceptual advantages relative to FP fusion methods, which have revolutionized the study of genetic expression,⁴³⁻⁴⁵ but have the well-known drawbacks that a new fusion must be engineered for each gene of interest, that it is difficult to determine whether fusions affect the expression or function of target proteins, and that fusion methods are not applicable to imaging non-protein gene products such as coding and non-coding RNAs. cgRNAs can eliminate these issues by replacing the conventional physical link of FP fusion approaches with a logical link executed by cgRNAs that execute conditional gene induction, allowing for spatiotemporal monitoring of gene expression levels in living chick embryos without the need to modify the imaged molecules (mRNA₁, mRNA₂, mRNA₃, mRNA₄) in any way. FIG. 3E depicts the conditional logic using cgRNAs as conditional chemotherapies: “if disease marker X then regulate therapeutic target Y”. Here, X is a programmable disease marker and Y is an independent therapeutic target, allowing for selective treatment or killing of diseased cells (the subset of cells containing X) while leaving healthy cells untouched (the subset of cells lacking X). In this way, cgRNAs allow for independent diagnosis (detection of disease marker X) and treatment (regulation or editing of independent therapeutic target Y).

### Example—Logic, Function, and Mechanism for Allosteric On→Off Terminator Switch cgRNAs (Mechanism 1A)

FIG. 4 depicts the logic, function, and mechanism for allosteric ON→OFF terminator switch cgRNAs. FIG. 4A depicts the conditional logic and function of an ON→OFF terminator switch cgRNA used in conjunction with different Cas protein effectors (for example, edit, silence, induce, bind): “if not X then regulate Y” (if trigger X is not detected, then edit, silence, induce, or bind target gene Y). FIGS. 4B and 4C depict a mechanism for an allosteric ON→OFF terminator switch cgRNA: the constitutively active cgRNA is inactivated by hybridization of RNA trigger X. This mechanism offers the flexibility to rationally design sequence domains (as well as their complementary domains) “d” (linker), “e” (stem), and “f” (extended loop) in the cgRNA, independent of the sequence of the target-binding region (domain “u”).

### Example—Logic, Function, and Mechanism for Allosteric OFF→ON Terminator Switch cgRNAs (Mechanism 1B)

FIG. 5 depicts the logic, function, and mechanism for allosteric OFF→ON terminator switch cgRNAs. FIG. 5A depicts the conditional logic and function of an OFF→ON terminator switch cgRNA used in conjunction with different Cas protein effectors (for example, edit, silence, induce, bind): “if X then regulate Y” (if trigger X is detected, then edit, silence, induce, or bind target gene Y depending on the Cas effector). FIGS. 5B and 5C depict a mechanism for an allosteric OFF→ON terminator switch cgRNA: the cgRNA:inhibitor complex is inactive; hybridization of RNA trigger X to the inhibitor displaces the inhibitor from the cgRNA, activating the cgRNA. This mechanism offers the flexibility to rationally design sequence domains (as well as their complementary domains) “d” (linker), “e” (stem), and “f” (extended loop) in the cgRNA, as well as toehold domains “g” and “h” in the trigger, independent of the sequence of the target-binding region (domain “u”).

### Example—Logic, Function, and Mechanisms for Allosteric ON→OFF Splinted Switch cgRNAs (Mechanism 2a)

FIG. 6 depicts the logic, function, and mechanism for allosteric ON→OFF splinted switch cgRNAs. FIG. 6A depicts the conditional logic and function of an ON→OFF splinted switch cgRNA used in conjunction with different Cas protein effectors (for example, edit, silence, induce, bind): “if not X then regulate Y” (if trigger X is not detected, then edit, silence, induce, or bind target gene Y depending on the Cas effector). FIGS. 6B and 6C depict a mechanism for an allosteric ON→OFF splinted switch cgRNA: the constitutively active cgRNA is inactivated by hybridization of RNA trigger X. This mechanism offers the flexibility to rationally design sequence domains (as well as their complementary domains) “d” (extended Cas9 handle loop) and “e” (extended terminator hairpin loop) in the cgRNA, independent of the sequence of the target-binding region (domain “u”).

### Example—Logic, Function, and Mechanisms for Allosteric OFF→ON Splinted Switch cgRNAs (Mechanism 2B)

FIG. 7 depicts the logic, function, and mechanism for allosteric OFF→ON splinted switch cgRNAs. FIG. 7A depicts the conditional logic and function of an OFF→ON splinted switch cgRNA used in conjunction with different Cas protein effectors (for example, edit, silence, induce, bind): “if X then regulate Y” (if trigger X is detected, then edit, silence, induce, or bind target gene Y depending on the Cas effector). FIGS. 7B and 7C depict a mechanism for an allosteric OFF→ON splinted switch cgRNA: the cgRNA:inhibitor complex is inactive; hybridization of RNA trigger X to the inhibitor displaces the inhibitor from the cgRNA, activating the cgRNA. This mechanism offers the flexibility to rationally design sequence domains (as well as their complementary domains) “d” (extended Cas9 handle loop) and “e” (extended terminator hairpin loop) in the cgRNA, as well as toehold domains “f” and “g” in the trigger, independent of the sequence of the target-binding region (domain “u”).

### Example—Logic, Function, and Mechanism for Allosteric ON→OFF Tandem Switch cgRNAs (Mechanism 3a)

FIG. 23 depicts the logic, function, and mechanism for allosteric ON→OFF tandem switch cgRNAs. FIG. 23A depicts the conditional logic and function of an ON→OFF tandem switch cgRNA used in conjunction with different Cas protein effectors (for example, edit, silence, induce, bind): “if not X then regulate Y” (if trigger X is not detected, then edit, silence, induce, or bind target gene Y). FIGS. 23B and 23C depict a mechanism for an allosteric ON→OFF tandem switch cgRNA: the constitutively active cgRNA is inactivated by hybridization of RNA trigger X. This mechanism offers the flexibility to rationally design sequence domains (as well as their complementary domains) “d” (extended Cas9 handle loop), “e” (linker), “f” (stem), and “g” (extended terminator hairpin loop) in the cgRNA, independent of the sequence of the target-binding region (domain “u”).

### Example—Logic, Function, and Mechanism for Allosteric OFF→ON Tandem Switch cgRNAs (Mechanism 3B)

FIG. 24 depicts the logic, function, and mechanism for allosteric OFF→ON tandem switch cgRNAs. FIG. 24A depicts the conditional logic and function of an OFF→ON tandem switch cgRNA used in conjunction with different Cas protein effectors (for example, edit, silence, induce, bind): “if X then regulate Y” (if trigger X is detected, then edit, silence, induce, or bind target gene Y depending on the Cas effector). FIGS. 24B and 24C depict a mechanism for an allosteric OFF→ON tandem switch cgRNA: the cgRNA:inhibitor complex is inactive; hybridization of RNA trigger X to the inhibitor displaces the inhibitor from the cgRNA, activating the cgRNA. This mechanism offers the flexibility to rationally design sequence domains (as well as their complementary domains) “d” (extended Cas9 handle loop), “e” (linker), “f” (stem), and “g” (extended terminator hairpin loop) in the cgRNA, as well as toehold domains “p” and “q” in the trigger, independent of the sequence of the target-binding region (domain “u”).

### Example—Logic, Function, and Mechanism for Allosteric OFF→ON Split-Terminator Switch cgRNAs (Mechanism 4a)

FIG. 8 depicts the logic, function, and mechanism for allosteric OFF→ON split-terminator switch cgRNAs. FIG. 8A depicts the conditional logic and function for an OFF→ON split-terminator switch cgRNA used in conjunction with different Cas protein effectors (for example, edit, silence, induce, bind): “if X then regulate Y” (if trigger X is detected, then edit, silence, induce, or bind target gene Y depending on the Cas effector). FIGS. 8B and 8C depict a mechanism for an allosteric OFF→ON split-terminator switch cgRNA: the constitutively inactive cgRNA is activated by hybridization of RNA trigger X. Equivalently, the cgRNA may be interpreted as a 5′ fragment (cg5) and the trigger may be interpreted as a 3′ fragment (cg3), such that cg5 and cg3 are inactive when not bound to each other, but such that upon binding to each other to form the complex cg5:cg3, this complex constitutes an activated conditional guide RNA capable of mediating Cas9 or dCas9 function. This mechanism offers the flexibility to rationally design the sequence of the terminator duplex (cgRNA domain “d” and trigger domain “d*”), independent of the sequence of the target-binding region (domain “u”).

### Example—Logic, Function, and Mechanisms for Allosteric ON→OFF Split-Terminator Switch cgRNA (Mechanisms 4B and 4C)

FIG. 9 depicts the logic, function, and mechanism for allosteric ON→OFF split-terminator switch cgRNAs. FIG. 9A depicts the conditional logic and function for an ON→OFF split-terminator switch cgRNA used in conjunction with different Cas protein effectors (for example, edit, silence, induce, bind): “if X then regulate Y” (if trigger X is detected, then edit, silence, induce, or bind target gene Y depending on the Cas effector). FIGS. 9B and 9C depict one mechanism for an allosteric ON→OFF split-terminator switch cgRNA (Mechanism 4B): the cgRNA: helper complex is active; hybridization of RNA trigger X to the cgRNA displaces the helper, inactivating the cgRNA. This mechanism offers the flexibility to rationally design the domains (as well as their complementary domains) “c” (linker), “d” (stem), and “e” (toehold) of the cgRNA. FIGS. 9D and 9E depict a second mechanism for an allosteric ON→OFF split-terminator switch cgRNA (Mechanism 4C): the cgRNA: helper complex is active; hybridization of RNA trigger X to the helper displaces the cgRNA, inactivating the cgRNA. This mechanism offers the flexibility to rationally design the terminator duplex (cgRNA domain “d” and RNA helper strand domains “d*”) as well as toehold domain “e*” on the RNA helper strand), independent of the sequence of the target-binding region (domain “u”).

### Example—Logic, Function, and Mechanism for Allosteric OFF→ON 5′-Inhibited Split-Terminator Switch cgRNAs (Mechanism 5)

FIG. 25 depicts the logic, function, and mechanism for allosteric OFF→ON 5′-inhibited split-terminator switch cgRNAs. FIG. 25A depicts the conditional logic and function for an OFF→ON 5′-inhibited split-terminator switch cgRNA used in conjunction with different Cas protein effectors (for example, edit, silence, induce, bind): “if X then regulate Y” (if trigger X is detected, then edit, silence, induce, or bind target gene Y depending on the Cas effector). FIGS. 25B and 25C depict a mechanism for an allosteric OFF→ON 5′-inhibited split-terminator switch cgRNA: in some embodiments, the constitutively inactive cgRNA comprises a 5′ fragment (cg5) and a 3′ fragment (cg3):
1. such that cg5 and cg3 are inactive when not bound to each other,
2. such that in the absence of a cognate RNA trigger, cg5 and cg3 are configured to be inhibited from binding to each other,
3. and such that upon hybridization of the cognate RNA trigger to cg5, cg5 and cg3 hybridize to form a trigger:cg5:cg3 complex that activates the cgRNA, mediating the function of a Cas protein effector on a target gene.

This mechanism offers the flexibility to rationally design sequence domains (as well as their complementary domains) “a”, “b”, and “c”, independent of the sequence of the target-binding region (domain “u”).

### Example—Logic, Function, and Mechanism for Allosteric OFF→ON 3′-Inhibited Split-Terminator Switch cgRNAs (Mechanism 6)

FIG. 26 depicts the logic, function, and mechanism for allosteric OFF→ON 3′-inhibited split-terminator switch cgRNAs. FIG. 26A depicts the conditional logic and function for an OFF→ON 3′-inhibited split-terminator switch cgRNA used in conjunction with different Cas protein effectors (for example, edit, silence, induce, bind): “if X then regulate Y” (if trigger X is detected, then edit, silence, induce, or bind target gene Y depending on the Cas effector). FIGS. 26B and 26C depict a mechanism for an allosteric OFF→ON 3′-inhibited split-terminator switch cgRNA: in some embodiments, the constitutively inactive cgRNA comprises a 5′ fragment (cg5) and a 3′ fragment (cg3):
1. such that cg5 and cg3 are inactive when not bound to each other,
2. such that in the absence of a cognate RNA trigger, cg5 and cg3 are configured to be inhibited from binding to each other,
3. and such that upon hybridization of the cognate RNA trigger to cg3, cg5 and cg3 hybridize to form a trigger:cg5:cg3 complex that activates the cgRNA, mediating the function of a Cas protein effector on a target gene.

This mechanism offers the flexibility to rationally design sequence domains (as well as their complementary domains) “a”, “b”, “c”, and “d”, independent of the sequence of the target-binding region (domain “u”).

### Example—Logic, Function, and Mechanism for Allosteric OFF→ON 5′- and 3′-Inhibited Split-Terminator Switch cgRNAs (Mechanism 7)

FIG. 27 depicts the logic, function, and mechanism for allosteric OFF→ON 5′- and 3′-inhibited split-terminator switch cgRNAs (Mechanism 7). FIG. 27A depicts the conditional logic and function for an OFF→ON 5′- and 3′-inhibited split-terminator switch cgRNA (Mechanism 7) used in conjunction with different Cas protein effectors (for example, edit, silence, induce, bind): “if X then regulate Y” (if trigger X is detected, then edit, silence, induce, or bind target gene Y depending on the Cas effector). FIGS. 27B and 27C depict a mechanism for an allosteric OFF→ON 5′- and 3′-inhibited split-terminator switch cgRNA (Mechanism 7): in some embodiments, the constitutively inactive cgRNA comprises a 5′ fragment (cg5) and a 3′ fragment (cg3):
1. such that cg5 and cg3 are inactive when not bound to each other,
2. such that in the absence of a cognate RNA trigger, cg5 and cg3 are configured to be inhibited from binding to each other,
3. and such that upon hybridization of the cognate RNA trigger to cg5, cg5 and cg3 hybridize to form a trigger:cg5:cg3 complex that activates the cgRNA, mediating the function of a Cas protein effector on a target gene.

In some embodiments, cg3 comprises a trigger mimic region with the same sequence as the cg5-binding region of the cognate RNA trigger, such that upon activation of the cgRNA by the cognate RNA trigger, the trigger mimic region is exposed and capable of serving as the cognate RNA trigger for a new copy of the cgRNA comprising a new copy of the 5′ fragment cg5 and a new copy of the 3′ fragment cg3. This mechanism offers the flexibility to rationally design sequence domains (as well as their complementary domains) “a”, “b”, “c”, and “d”, independent of the sequence of the target-binding region (domain “u”).

### Example—Logic, Function, and Mechanism for Allosteric OFF→ON 5′- and 3′-Inhibited Split-Terminator Switch cgRNAs (Mechanism 8)

FIG. 28 depicts the logic, function, and mechanism for allosteric OFF→ON 5′- and 3′-inhibited split-terminator switch cgRNAs (Mechanism 8). FIG. 28A depicts the conditional logic and function for an OFF→ON 5′- and 3′-inhibited split-terminator switch cgRNA (Mechanism 8) used in conjunction with different Cas protein effectors (for example, edit, silence, induce, bind): “if X then regulate Y” (if trigger X is detected, then edit, silence, induce, or bind target gene Y depending on the Cas effector). FIGS. 28B and 28C depict a mechanism for an allosteric OFF→ON 5′- and 3′-inhibited split-terminator switch cgRNA (Mechanism 8): in some embodiments, the constitutively inactive cgRNA comprises a 5′ fragment (cg5) and a 3′ fragment (cg3):
1. such that cg5 and cg3 are inactive when not bound to each other,
2. such that in the absence of a cognate RNA trigger, cg5 and cg3 are configured to be inhibited from binding to each other,
3. and such that upon hybridization of the cognate RNA trigger to cg5, cg5 and cg3 hybridize to form a trigger:cg5:cg3 complex that activates the cgRNA, mediating the function of a Cas protein effector on a target gene.

In some embodiments, the cgRNA additionally comprises a splint fragment such that in the absence of the cognate RNA trigger the splint fragment is configured to be inhibited from binding cg5 and cg3, and such that upon activation of the cgRNA by the cognate RNA trigger, cg3 hybridizes to the split fragment which then hybridizes to cg5, displacing the trigger from cg5 to catalytically regenerate the trigger which can then serve as the cognate RNA trigger for a new copy of the cgRNA comprising a new copy of the 5′ fragment cg5, a new copy of the 3′ fragment cg3, and a new copy of the splint fragment. This mechanism offers the flexibility to rationally design sequence domains (as well as their complementary domains) “a”, “b”, and “c”, independent of the sequence of the target-binding region (domain “u”).

### Example—Logic, Function, and Mechanism for Allosteric OFF→ON 5′- and 3′-Inhibited Split-Terminator Switch cgRNAs (Mechanism 9)

FIG. 29 depicts the logic, function, and mechanism for allosteric OFF→ON 5′- and 3′-inhibited split-terminator switch cgRNAs (Mechanism 9). FIG. 29A depicts the conditional logic and function for an OFF→ON 5′- and 3′-inhibited split-terminator switch cgRNA (Mechanism 9) used in conjunction with different Cas protein effectors (for example, edit, silence, induce, bind): “if X then regulate Y” (if trigger X is detected, then edit, silence, induce, or bind target gene Y depending on the Cas effector). FIGS. 29B and 29C depict a mechanism for an allosteric OFF→ON 5′- and 3′-inhibited split-terminator switch cgRNA (Mechanism 9): in some embodiments, the constitutively inactive cgRNA comprises a 5′ fragment (cg5) and a 3′ fragment (cg3):
1. such that cg5 and cg3 are inactive when not bound to each other,
2. such that in the absence of a cognate RNA trigger, cg5 and cg3 are configured to be inhibited from binding to each other,
3. and such that upon hybridization of the cognate RNA trigger to cg5, cg5 and cg3 are hybridize to form a trigger:cg5:cg3 complex that activates the cgRNA, mediating the function of a Cas protein effector on a target gene.

In some embodiments, cg3 additionally comprises a trigger mimic region such that upon activation of the cgRNA by the cognate RNA trigger, the trigger mimic region hybridizes to cg5 to displace the trigger, catalytically regenerating the trigger which can then serve as the cognate RNA trigger for a new copy of the cgRNA comprising a new copy of the 5′ fragment cg5 and a new copy of the 3′ fragment cg3. This mechanism offers the flexibility to rationally design sequence domains (as well as their complementary domains) “a”, “b”, and “c”, independent of the sequence of the target-binding region (domain “u”).

### Example—Depictions of Interactions Between Allosteric cgRNAs, RNA Triggers, and Cas9 or dCas9

FIG. 10 depicts interactions between allosteric cgRNAs, RNA triggers, and Cas9, dCas9 or Cas. FIG. 10A depicts interactions for an allosteric ON→OFF terminator switch cgRNA. In the ON state, the terminator switch cgRNA is constitutively active, directing the function of protein effector Cas9, dCas9, or Cas to a target gene Y in the absence of trigger. The extended loop and modified sequence domains in the terminator region are intended not to interfere with the activity of the cgRNA: Cas complex. In the OFF state, in the presence of RNA trigger X, hybridization of the trigger forms a structure incompatible with cgRNA mediation of Cas9, dCas9, and/or Cas function. FIG. 10B depicts interactions for an allosteric ON→OFF splinted switch cgRNA. In the ON state, the splinted switch cgRNA is constitutively active, directing the function of protein effector Cas9, dCas9, or Cas to a target gene Y in the absence of trigger. The extended loops in the Cas9 handle and terminator region are intended not to interfere with the activity of the cgRNA: Cas complex. In the OFF state, in the presence of RNA trigger X, hybridization of the trigger forms a splint that is structurally incompatible with cgRNA mediation of Cas9, dCas9, and/or Cas function. FIG. 10C depicts interactions for an allosteric OFF→ON split-terminator switch cgRNA. In the OFF state, the split-terminator switch cgRNA is constitutively inactive. In the absence of RNA trigger X, the cgRNA is incapable of directing the function of the protein effector Cas9, dCas9, and/or Cas. In the ON state, the complex of cgRNA and trigger X mediates the function of the protein effector Cas9, dCas9, or Cas on the target gene Y. The modified sequence domains in the terminator duplex do not to interfere with the activity of the cgRNA: trigger: Cas complex.

### Example—Demonstration of Allosteric ON→OFF Terminator Switch cgRNAs in Bacteria

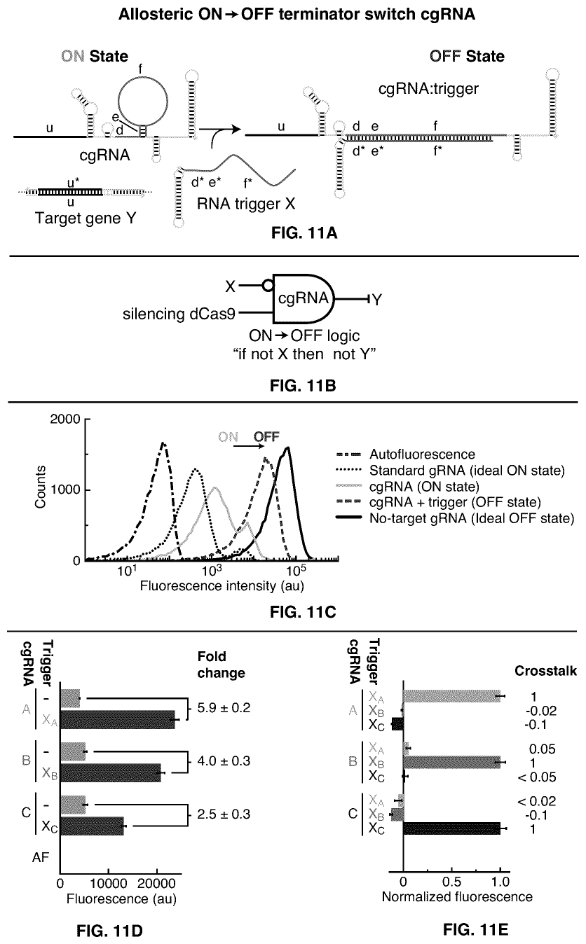

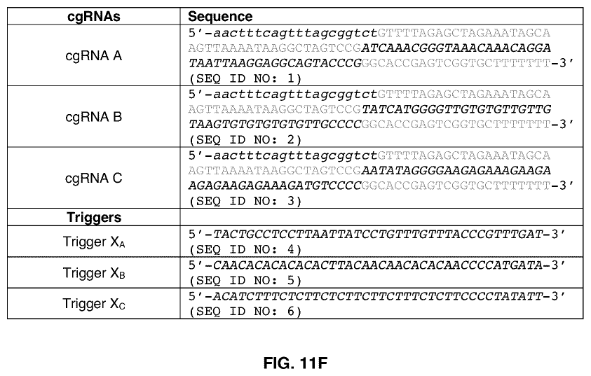

FIG. 11 demonstrates allosteric ON→OFF terminator switch cgRNAs performing conditional logic in E. coli. FIG. 11A depicts the mechanism for an allosteric ON→OFF terminator switch cgRNA: the constitutively active cgRNA is inactivated by hybridization of RNA trigger X. Rational sequence design of cgRNA terminator region (domains “d-e-f” comprising 6 nt linker, 4 nt stem, 30 nt loop) and complementary trigger region (domains “f*-e*-d*”). FIG. 11B depicts the conditional logic for an ON→OFF terminator switch cgRNA used in conjunction with silencing dCas9: “if not X then not Y” (if trigger X is not detected, then silence target gene Y). FIG. 11C demonstrates that expression of RNA trigger X (40 nt unstructured+synthetic terminator hairpin) toggles the cgRNA from ON→OFF, leading to an increase in fluorescence. Single-cell fluorescence intensities via flow cytometry. Induced expression (aTc) of silencing dCas9 and constitutive expression of mRFP target gene Y and either: standard gRNA (ideal ON state), cgRNA (ON state), cgRNA+RNA trigger X (OFF state; trigger expression is IPTG-induced), no-target gRNA that lacks target-binding region (ideal OFF state). Autofluorescence (AF): cells with no mRFP. FIGS. 11D and 11E demonstrate programmable conditional regulation using 3 orthogonal cgRNAs (A, B, C) and their corresponding cognate triggers (X_{A}, X_{B}, X_{C}). In FIG. 11D, raw fluorescence depicts ON→OFF conditional response to cognate trigger. Fold change=OFF/ON=[cognate trigger AF]/[no trigger-AF]). In FIG. 11E, normalized fluorescence depicts orthogonality between non-cognate cgRNA/trigger pairs. Crosstalk=[non-cognate trigger-no trigger]/[cognate trigger-no trigger]). Bar graphs depict mean±estimated standard error calculated based on the mean single-cell fluorescence over 20,000 cells for each of N=3 replicate wells (OFF:ON ratio and crosstalk calculated with uncertainty propagation). FIG. 11F depicts the sequences of cgRNAs A, B, C, and the sequences of triggers X_{A}, X_{B}, X_{C}. Nucleotides that are lower case italic are constrained by the target gene. Nucleotides shaded gray are constrained by dCas9. Nucleotides that are upper case italic are designed. Sequences are presented as DNA sequences; the corresponding RNA sequence is the same as the DNA sequence except that “U” replaces “T”).

In E. coli expressing an allosteric ON→OFF terminator switch (FIG. 11A), silencing dCas9 and a fluorescent protein reporter (mRFP) as the target gene Y (conditional logic: “if not X then not Y”; FIG. 11B), the cgRNA exhibits a strong conditional response to expression of RNA trigger X (FIG. 11C). FIG. 11D displays raw fluorescence data for a library of three orthogonal ON→OFF splinted switch cgRNAs with and without their cognate triggers. For this library of three cgRNA/trigger pairs, the median fold-change is approximately 4× for the ON→OFF conditional response to expression of the cognate trigger (FIG. 11D) and the median crosstalk between non-cognate/trigger pairs is approximately 2% (FIG. 11E).

Orthogonal cgRNA/trigger pairs were designed using NUPACK.^{40,41} cgRNA/trigger plasmids were transformed into a modified E. coli MG1655 strain expressing genomically incorporated mRFP and sfGFP4.⁵ Strains were grown overnight in EZ-RDM (Teknova), then diluted and grown to midlog phase (approximately 4 h). Cell density was normalized with fresh medium containing aTc for induction of silencing dCas9 expression and IPTG. Induced cells were grown for 12 h, with end-point fluorescence measured via flow cytometry.

### Example—Demonstration of Allosteric ON→OFF Splinted Switch cgRNAs in Bacteria

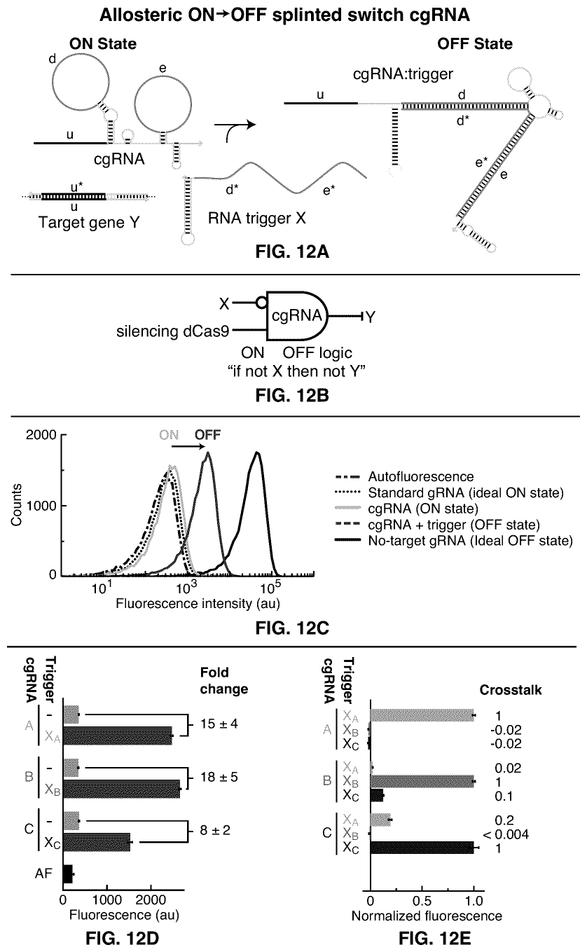

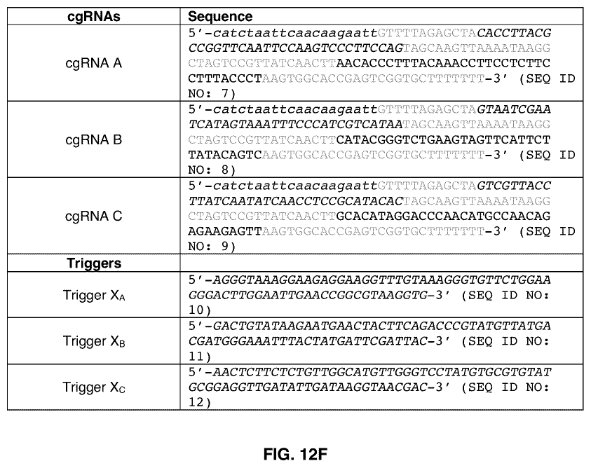

FIG. 12 demonstrates allosteric ON→OFF splinted switch cgRNAs performing conditional logic in E. coli. FIG. 12A depicts the mechanism for an allosteric ON→OFF splinted switch cgRNA: the constitutively active cgRNA is inactivated by hybridization of RNA trigger X. Rational sequence design of the 35 nt Cas9 handle loop (domain “d”) and an extended 35 nt terminator hairpin loop (domain “e”). FIG. 12B depicts the conditional logic for an ON→OFF splinted switch cgRNA used in conjunction with silencing dCas9: “if not X then not Y” (if trigger X is not detected, then silence target gene Y). FIG. 12C demonstrates that expression of RNA trigger X (70 nt unstructured+synthetic terminator hairpin) toggles the cgRNA from ON→OFF, leading to an increase in fluorescence. Single-cell fluorescence intensities via flow cytometry. Induced expression (aTc) of silencing dCas9 and constitutive expression of sfGFP target gene Y and either: standard gRNA (ideal ON state), cgRNA (ON state), cgRNA+RNA trigger X (OFF state), no-target gRNA that lacks target-binding region (ideal OFF state). Autofluorescence (AF): cells with no sfGFP. FIGS. 12D and 12E demonstrate programmable conditional regulation using 3 orthogonal cgRNAs (A, B, C) and their corresponding cognate triggers (X_{A}, X_{B}, X_{C}). In FIG. 12D, raw fluorescence depicts ON→OFF conditional response to cognate trigger. Fold change=OFF/ON=[cognate trigger−AF]/[no trigger−AF]). In FIG. 12E, normalized fluorescence depicts orthogonality between non-cognate cgRNA/trigger pairs. Crosstalk=[non−cognate trigger-no trigger]/[cognate trigger−no trigger]). Bar graphs depict mean±estimated standard error calculated based on the mean single-cell fluorescence over 20,000 cells for each of N=3 replicate wells (OFF:ON ratio and crosstalk calculated with uncertainty propagation). FIG. 12F depicts the sequences of cgRNAs A, B, C, and the sequences of triggers X_{A}, X_{B}, X_{C}. Nucleotides that are lower case italic are constrained by the target gene. Nucleotides shaded gray are constrained by dCas9. Nucleotides that are upper case italic are designed. Sequences are presented as DNA sequences; the corresponding RNA sequence is the same as the DNA sequence except that “U” replaces “T”).

In E. coli expressing an allosteric ON→OFF splinted switch (FIG. 12A), silencing dCas9 and a fluorescent protein reporter (sfGFP) as the target gene Y (conditional logic: “if not X then not Y”; FIG. 12B), the cgRNA exhibits a strong conditional response to expression of RNA trigger X (FIG. 12C). The ON state approaches the ideal ON state of a standard unconditional gRNA. FIG. 12D displays raw fluorescence data for a library of three orthogonal ON→OFF splinted switch cgRNAs with and without their cognate triggers. For this library of three cgRNA/trigger pairs, the median fold-change is approximately 15× for the ON→OFF conditional response to expression of the cognate trigger (FIG. 12D) and the median crosstalk between non-cognate/trigger pairs is approximately 2% (FIG. 12E).

Orthogonal cgRNA/trigger pairs were designed using NUPACK.^{40,41} cgRNA/trigger plasmids were transformed into a modified E. coli MG1655 strain expressing genomically incorporated mRFP and sfGFP4.⁵ Strains were grown overnight in EZ-RDM (Teknova), then diluted and grown to midlog phase (approximately 4 h). Cell density was normalized with fresh medium containing aTc for induction of silencing dCas9 expression. Induced cells were grown for 12 h, with end-point fluorescence measured via flow cytometry.

### Example—Demonstration of Allosteric ON→OFF Terminator Switch cgRNAs in Human Cells

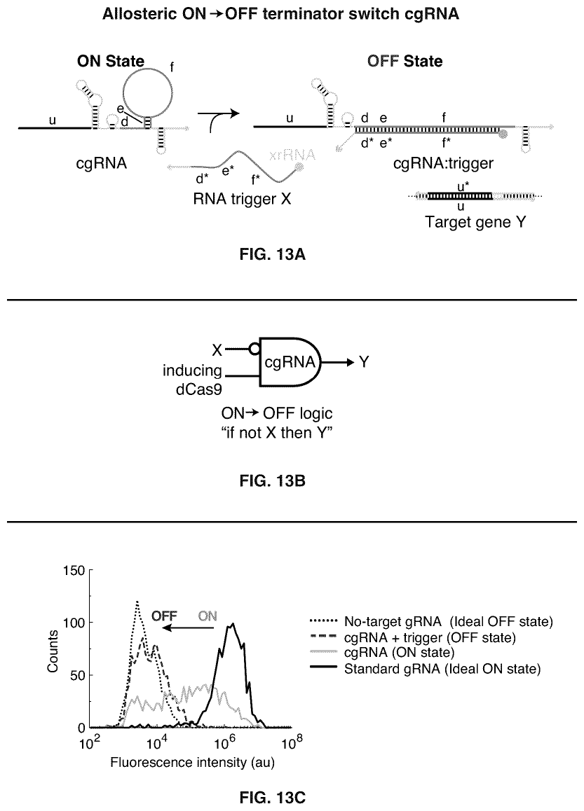

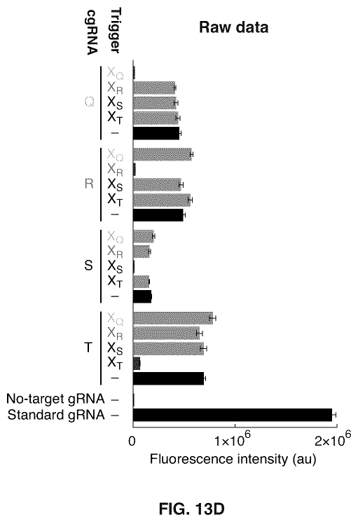

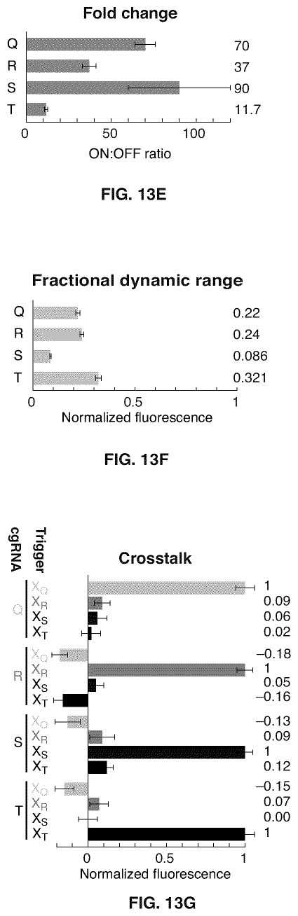

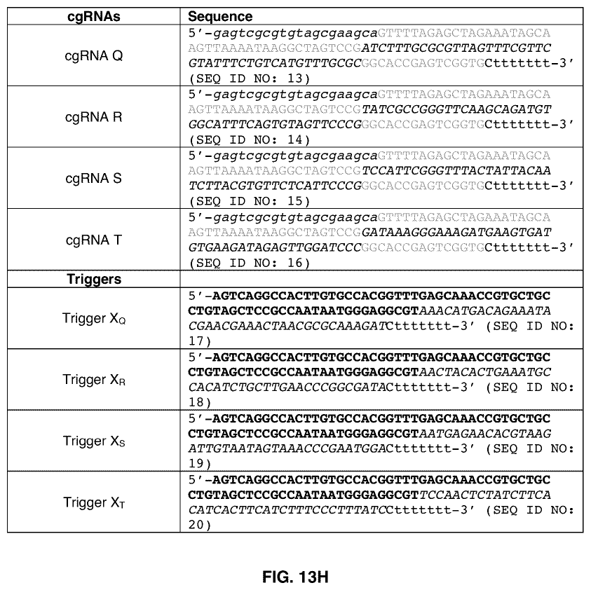

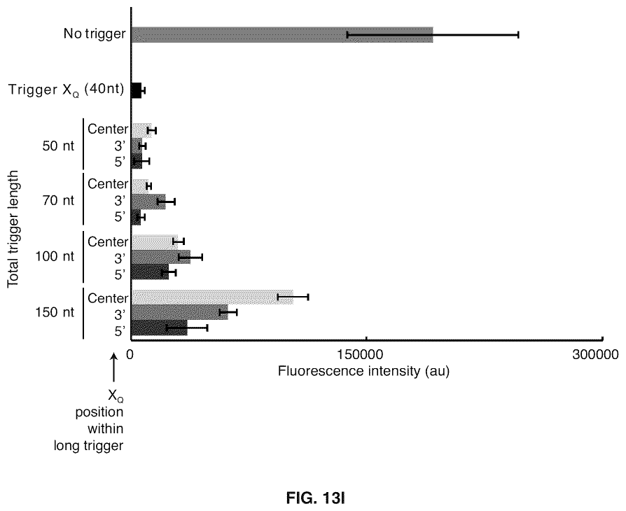

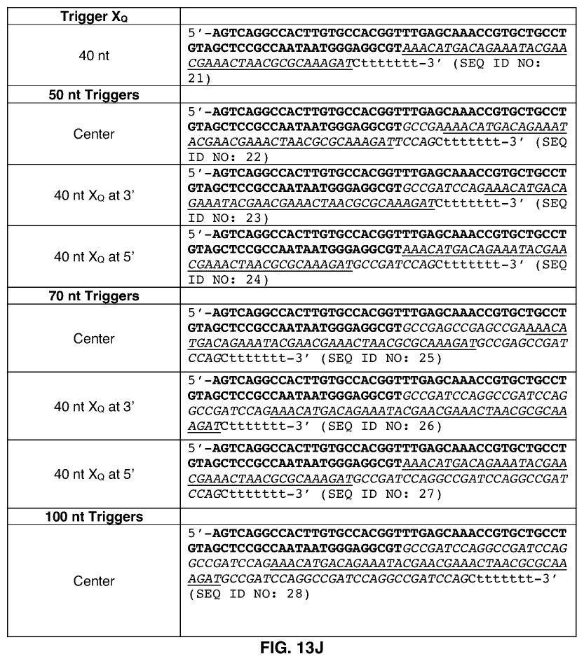

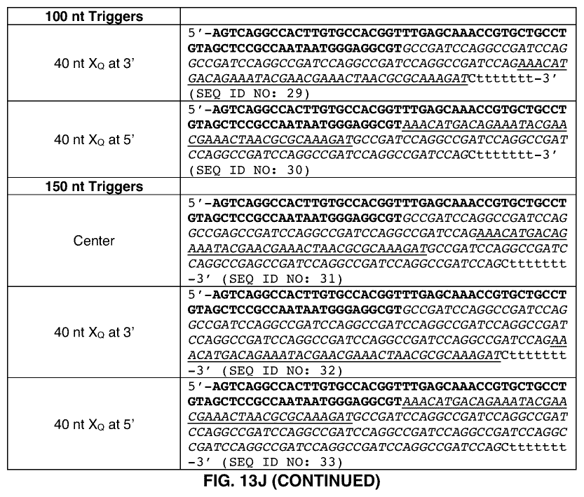

FIG. 13 demonstrates allosteric ON→OFF terminator switch cgRNAs performing conditional logic in HEK 293T cells. FIG. 13A depicts the mechanism for an allosteric ON→OFF terminator switch cgRNA: the constitutively active cgRNA is inactivated by hybridization of RNA trigger X. Rational design of cgRNA terminator region (domains “d-e-f”: 6 nt linker, 4 nt stem, 30 nt loop for FIGS. 13C-13H; 6 nt linker, 4 nt stem, 30 nt, 40 nt, 60 nt, 90 nt, or 140 nt loop for FIGS. 13I-13J) and complementary trigger region (domains “f*-e*-d*”). FIG. 13B depicts the conditional logic for an ON→OFF terminator switch cgRNA used in conjunction with inducing dCas9: “if not X then Y” (if trigger X is not detected, induce target gene Y). FIG. 13C demonstrates that expression of RNA trigger X (PEL+40 nt unstructured+hU6 terminator) toggles the cgRNA from ON→OFF, leading to a decrease in fluorescence. Single-cell fluorescence intensities via flow cytometry. Transfection of plasmids expressing inducing dCas9-VPR, Phi-YFP target gene Y, and either: standard gRNA+no-trigger control (ideal ON state), cgRNA+no-trigger control (ON state), cgRNA+RNA trigger X (OFF state), no-target gRNA that lacks target-binding region+no-trigger control (ideal OFF state). The no-trigger control uses a random pool of triggers to provide a sequence-generic approximation of the metabolic load of trigger expression. FIGS. 13D-13G demonstrate programmable conditional regulation using a library of 4 orthogonal cgRNAs (Q, R, S, T) and their corresponding cognate triggers (X_{Q}, X_{R}, X_{S}, X_{T}). In FIG. 13D, raw fluorescence depicts ON→OFF conditional response to cognate trigger. In FIG. 13E, fold change=ON/OFF. In FIG. 13F, fractional dynamic range=(ON−OFF)/(ideal ON−ideal OFF). In FIG. 13G, crosstalk=(ON−OFF′)/(ON−OFF) where OFF′ corresponds to cgRNA+non-cognate trigger. Bar graphs depict mean±estimated standard error of the mean (with uncertainty propagation) calculated based on the mean single-cell fluorescence over 1545-3970 cells for each of N=3 replicate wells.

FIG. 13I demonstrates a conditional ON→OFF cgRNA response to RNA triggers of varying lengths. Expression of RNA trigger X (PEL+ [40 nt, 50 nt, 70 nt, 100 nt, or 150 nt unstructured]+hU6 terminator) toggles the cgRNA Q from ON□OFF, leading to a decrease in fluorescence. For these studies, the 40 nt trigger sequence X_{Q} is expressed at either the 5′ end, 3′end, or in the middle of a 50 nt, 70 nt, 100 nt, or 150 nt sequence designed to have minimal structure. Bar graphs depict mean±estimated standard error of the mean (with uncertainty propagation) calculated based on the mean single-cell fluorescence over 1993-20393 cells for each of N=4 replicate wells.

FIG. 13H depicts the sequences of cgRNAs Q, R, S, T, and the sequences of triggers X_{Q}, X_{R}, X_{S}, X_{T} for the studies of FIGS. 13C-13G; FIG. 13J depicts the sequences of the long triggers that incorporate X_{Q} for the studies of FIG. 13I. Nucleotides that are lower case italic are constrained by the target binding site on the reporter plasmid. Nucleotides shaded gray are constrained by dCas9. Nucleotides that are upper case italic are designed. In FIG. 13I, underlined nucleotides depict the location of the X_{Q} sequence within the context of the longer trigger sequence. The plain “C” nucleotide is a cloning artifact. Lower case plain nucleotides are constrained by the hU6 terminator sequence⁴⁶. Bold nucleotides are a protective element (PEL) constrained by an xrRNA sequence derived from Dengue (Dengue 4, NC_002640.1)⁴⁷. Sequences are presented as DNA sequences; the corresponding RNA sequence is the same as the DNA sequence except that “U” replaces “T”).

In HEK 293T cells expressing an allosteric ON→OFF terminator switch cgRNA (FIG. 13A), inducing dCas9-VPR as the protein effector,⁴⁸ and a fluorescent protein reporter (Phi-YFP)^{49,50} as the target gene Y (conditional logic: “if not X then Y”; FIG. 13B), the cgRNA exhibits a strong conditional response to expression of the RNA trigger X (FIG. 13C). The OFF state approaches the ideal OFF state of a no-target gRNA lacking the target-binding region. FIG. 13D displays raw fluorescence data for a library of four orthogonal ON→OFF terminator switch cgRNAs with each of four triggers. For each of four cgRNAs, the cognate trigger yields low fluorescence (OFF state) comparable to the ideal OFF state using a no-target gRNA lacking the target-binding region, while each of three non-cognate triggers yields high fluorescence (ON state) comparable to a no-trigger control. For this library of four cgRNA/trigger pairs, the median fold-change is approximately 50× for the ON→OFF conditional response to expression of the cognate trigger (FIG. 13E); the median fractional dynamic range is approximately 20% (FIG. 13F); the median crosstalk between non-cognate/trigger pairs is approximately 4%, and the median crosstalk modulus is approximately 9%.

To optimize fold-change, the goal is to maximize the ON→OFF or OFF→ON conditional response ratio with/without the cognate RNA trigger (higher is better). To optimize fractional dynamic range, the goal is to maximize the difference between conditional ON and OFF states as a fraction of the unconditional regulatory dynamic range of CRISPR/Cas using standard gRNAs (higher is better). To optimize crosstalk, the goal is to minimize sequence (and metabolic) interactions between cgRNAs and non-cognate triggers including the transcriptome (lower is better). The orthogonal cgRNA/trigger pairs were designed using NUPACK^{40,41}. A cgRNA expression plasmid and a trigger expression plasmid were co-transfected with a plasmid expressing an inducing dCas9-VPR fusion⁴⁸ and a reporter plasmid containing a gRNA binding site upstream of a minimal CMV promoter for Phi-YFP expression.^{51,52} The four plasmids were transiently transfected into HEK 293T cells with Lipofectamine 2000 and grown for 24 h, with end-point fluorescence measured via flow cytometry. Data analysis was performed on cells expressing high levels of both cgRNA and trigger fluorescent protein transfection controls.

### Example—Demonstration of Allosteric OFF→ON Split-Terminator Switch cgRNAs in Human Cells

FIG. 14 demonstrates allosteric OFF→ON split-terminator switch cgRNAs performing conditional logic in HEK 293T cells. FIG. 14A depicts the mechanism for an allosteric OFF→ON split-terminator switch cgRNA: the constitutively inactive cgRNA is activated by hybridization of RNA trigger X. Rational design of 4 bp terminator duplex (cgRNA domain “d” and trigger domain “d*”). FIG. 14B depicts the conditional logic for an OFF→ON split-terminator switch cgRNA used in conjunction with inducing dCas9: “if X then Y” (if trigger X is detected, induce target gene Y). FIG. 14C demonstrates that expression of RNA trigger X (4 nt+terminator hairpin) toggles the cgRNA from OFF→ON, leading to an increase in fluorescence. Single-cell fluorescence intensities via flow cytometry. Transfection of plasmids expressing inducing dCas9-VPR, Phi-YFP target gene Y, and either: no-target gRNA that lacks target-binding region+no-trigger control (ideal OFF state), cgRNA+no-trigger control (OFF state), cgRNA+RNA trigger X (ON state), standard gRNA+no-trigger control (ideal ON state). FIGS. 14D-14G demonstrate programmable conditional regulation using a library of 3 orthogonal cgRNAs (M,N,O) and their corresponding cognate triggers (X_{M}, X_{N}, X_{O}). In FIG. 14D, raw fluorescence depicts OFF→ON conditional response to cognate trigger. In FIG. 14E, fold change=ON/OFF. In FIG. 14F, fractional dynamic range=(ON−OFF)/(ideal ON−ideal OFF). In FIG. 14G, crosstalk=(ON′−OFF)/(ON−OFF) where ON′ corresponds to cgRNA+non-cognate trigger. Bar graphs depict mean±estimated standard error of the mean (with uncertainty propagation) calculated based on the mean single-cell fluorescence over 1017-2394 cells for each of N=3 replicate wells. FIG. 14H depicts the sequences of cgRNAs M,N,O, and the sequences of triggers X_{M}, X_{N}, X_{O}. Nucleotides that are lower case italic are constrained by the target binding site on the reporter plasmid. Nucleotides shaded gray are constrained by dCas9. Nucleotides that are upper case italic are designed. The plain “C” nucleotide is a cloning artifact. Lower case plain nucleotides are constrained by the hU6 terminator sequence⁴⁶. Sequences are presented as DNA sequences; the corresponding RNA sequence is the same as the DNA sequence except that “U” replaces “T”). The orthogonal cgRNA/trigger pairs were designed using NUPACK^{40,41}.

In HEK 293T cells expressing an allosteric OFF→ON split-terminator switch cgRNA (FIG. 14A), inducing dCas9-VPR as the protein effector,⁴⁸ and a fluorescent protein reporter (Phi-YFP)^{49,50} as the target gene Y (conditional logic “if X then Y”; FIG. 14B), the cgRNA exhibits a strong conditional OFF→ON response to expression of the RNA trigger X (FIG. 14C). The OFF state approaches the ideal OFF state of a no-target gRNA lacking the target-binding region. FIG. 14D displays raw fluorescence data for a library of three orthogonal OFF→ON split-terminator switch cgRNAs with each of three triggers. The three cgRNAs have clean OFF states (low fluorescence) in the absence of trigger and strong ON states (high fluorescence) in response to expression of the cognate trigger. For this library of three cgRNA/trigger pairs, the median fold-change is approximately 150× for the conditional OFF→ON response to expression of the cognate trigger (FIG. 14E); the median fractional dynamic range is approximately 50% (FIG. 14F); the median crosstalk is approximately 4% (FIG. 14G).

To optimize fold-change, the goal is to maximize the ON→OFF or OFF→ON conditional response ratio with/without the cognate RNA trigger (higher is better). To optimize fractional dynamic range, the goal is to maximize the difference between conditional ON and OFF states as a fraction of the unconditional regulatory dynamic range of CRISPR/Cas using standard gRNAs (higher is better). To optimize crosstalk, the goal is to minimize sequence (and metabolic) interactions between cgRNAs and non-cognate triggers including the transcriptome (lower is better). The orthogonal cgRNA/trigger pairs were designed using NUPACK^{40,41}. A cgRNA expression plasmid and a trigger expression plasmid were co-transfected with a plasmid expressing an inducing dCas9-VPR fusion⁴⁸ and a reporter plasmid containing a gRNA binding site upstream of a minimal CMV promoter for Phi-YFP expression.^{51,52} The four plasmids were transiently transfected into HEK 293T cells with Lipofectamine 2000 and grown for 24 h, with end-point fluorescence measured via flow cytometry. Data analysis was performed on cells expressing high levels of both cgRNA and trigger fluorescent protein transfection controls.

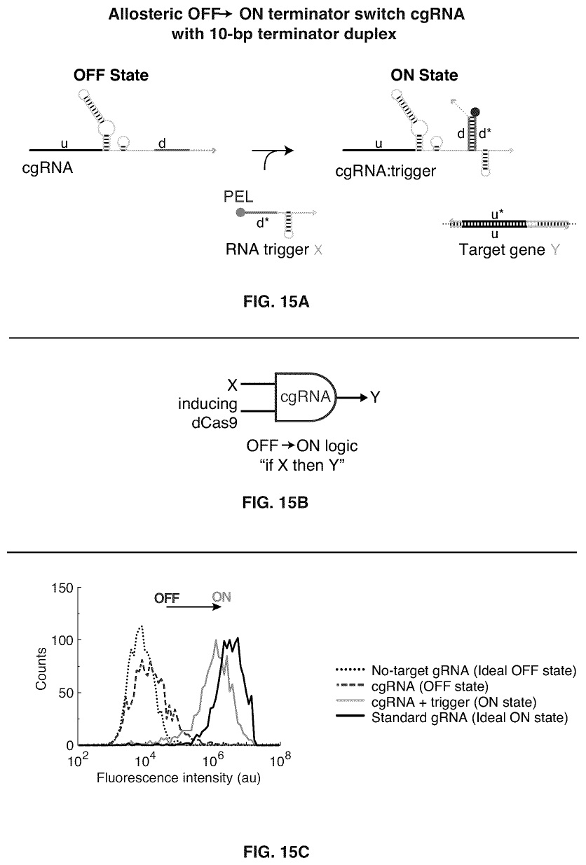

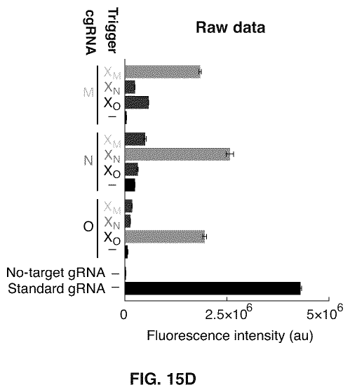

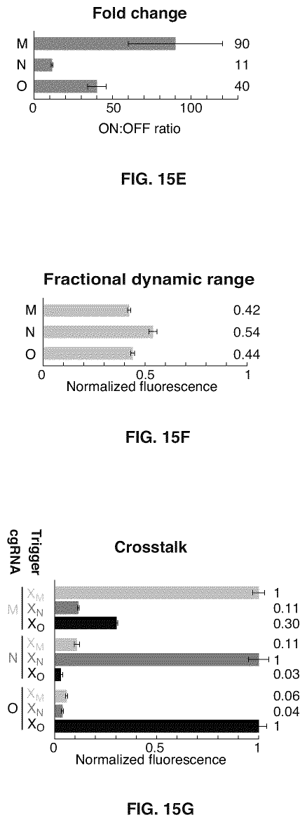

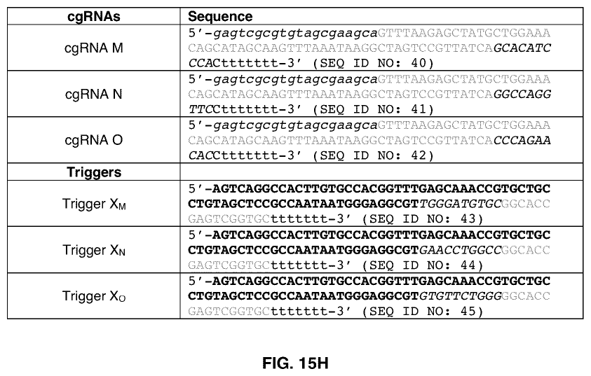

FIG. 14A-14H described above demonstrate split-terminator switch cgRNAs functioning in human cells using a 4 bp terminator duplex (formed via hybridization between domain “d” of the cgRNA and the reverse complementary domain “d*” in the trigger; see the mechanism schematic of FIG. 14A with domain lengths |d|=|d*|=4 nt). FIG. 15A-15H demonstrate that mechanism functioning using a 10 bp terminator duplex (see the mechanism schematic of FIG. 15A with domain lengths |d|=|d*|=10 nt).

FIG. 15A depicts the mechanism for an allosteric OFF→ON split-terminator switch cgRNA: the constitutively inactive cgRNA is activated by hybridization of RNA trigger X. Rational design of 10 bp terminator duplex (cgRNA domain “d” and trigger domain “d*”). FIG. 15B depicts the conditional logic for an OFF→ON split-terminator switch cgRNA using in conjunction with inducing dCas9: “if X then Y” (if trigger X is detected, induce target gene Y). FIG. 15C demonstrates that expression of RNA trigger X (PEL+10 nt+terminator hairpin) toggles the cgRNA from OFF→ON, leading to an increase in fluorescence. Single-cell fluorescence intensities via flow cytometry. Transfection of plasmids expressing inducing dCas9-VPR, Phi-YFP target gene Y, and either: no-target gRNA that lacks target-binding region+no-trigger control (ideal OFF state), cgRNA+no-trigger control (OFF state), cgRNA+RNA trigger X (ON state), standard gRNA+no-trigger control (ideal ON state). FIGS. 15D-15G demonstrate programmable conditional regulation using a library of 3 orthogonal cgRNAs (M,N,O) and their corresponding cognate triggers (X_{M}, X_{N}, X_{O}). In FIG. 15D, raw fluorescence depicts OFF→ON conditional response to cognate trigger. In FIG. 15E, fold change=ON/OFF. In FIG. 15F, fractional dynamic range=(ON−OFF)/(ideal ON−ideal OFF). In FIG. 15G, crosstalk=(ON′−OFF)/(ON−OFF) where ON′ corresponds to cgRNA+non-cognate trigger. Bar graphs depict mean+estimated standard error of the mean (with uncertainty propagation) calculated based on the mean single-cell fluorescence over 1244-2313 cells for each of N=3 replicate wells. FIG. 15H depicts the sequences of cgRNAs M,N,O, and the sequences of triggers X_{M}, X_{N}, X_{O}. Nucleotides that are lower case italic are constrained by the target binding site on the reporter plasmid. Nucleotides shaded gray are constrained by dCas9. Nucleotides that are upper case italic are designed. The plain “C” nucleotide is a cloning artifact. Lower case plain nucleotides are constrained by the hU6 terminator sequence⁴⁶. Bold nucleotides are a protective element (PEL) constrained by an xrRNA sequence derived from Dengue (Dengue 4, NC_002640.1)⁴⁷. Sequences are presented as DNA sequences; the corresponding RNA sequence is the same as the DNA sequence except that “U” replaces “T”). The orthogonal cgRNA/trigger pairs were designed using NUPACK^{40,41}. A cgRNA expression plasmid and a trigger expression plasmid were co-transfected with a plasmid expressing an inducing dCas9-VPR fusion⁴⁸ and a reporter plasmid containing a gRNA binding site upstream of a minimal CMV promoter for Phi-YFP expression.^{51,52} The four plasmids were transiently transfected into HEK 293T cells with Lipofectamine 2000 and grown for 24 h, with end-point fluorescence measured via flow cytometry. Data analysis was performed on cells expressing high levels of both cgRNA and trigger fluorescent protein transfection controls.

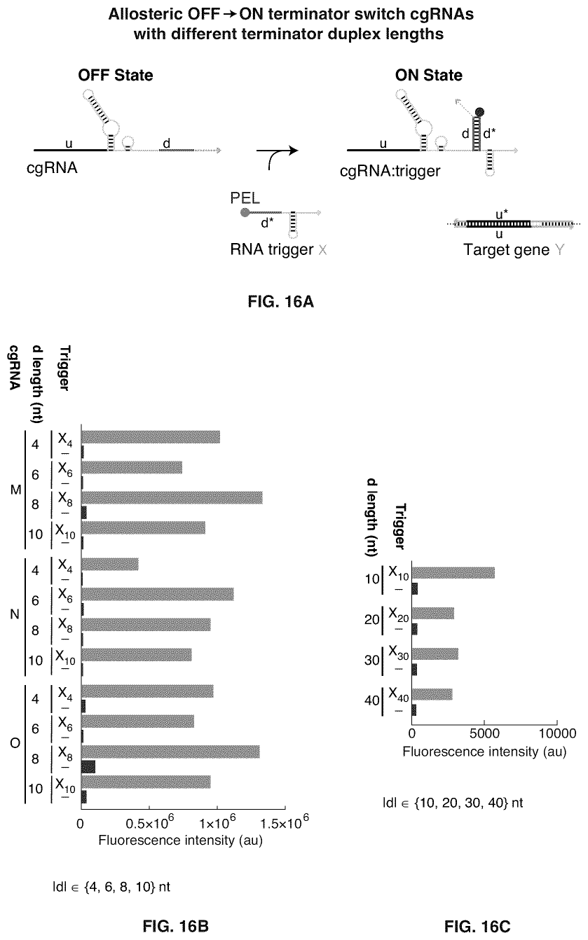

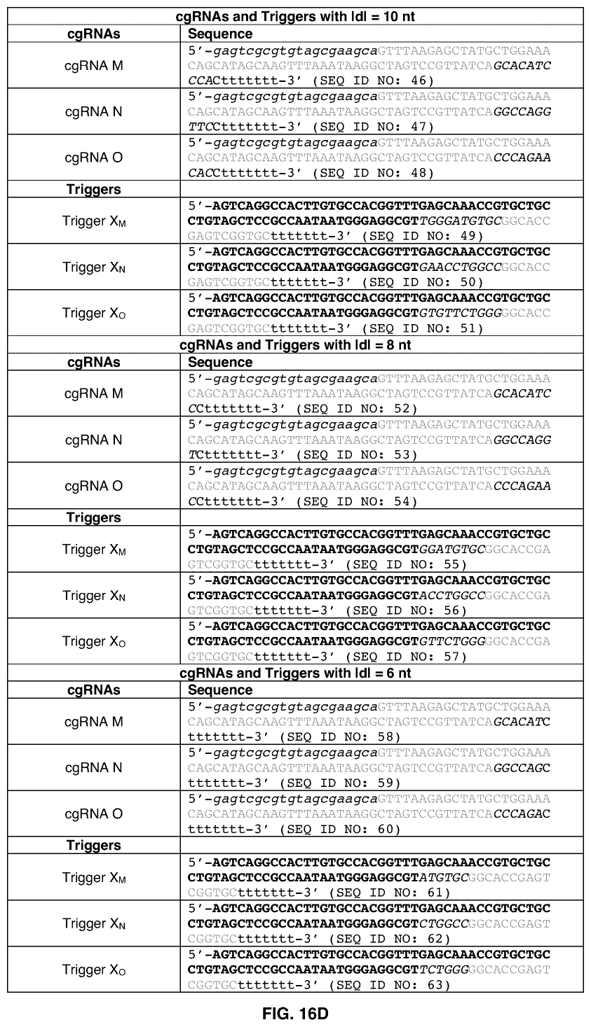

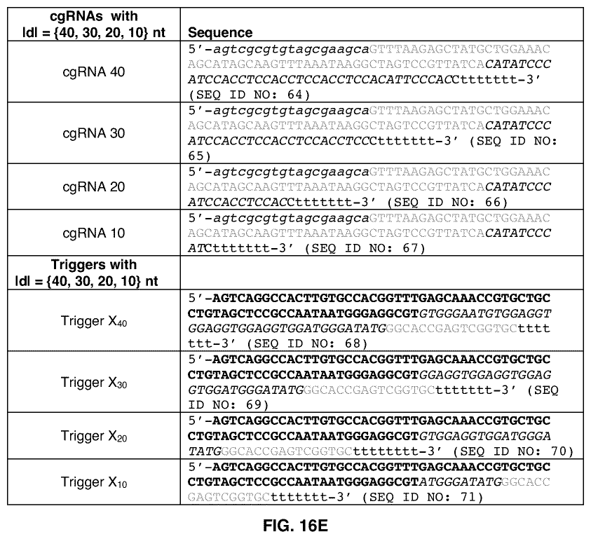

FIG. 16 demonstrates split-terminator switch cgRNAs functioning in HEK293T cells using terminator duplexes of different lengths ranging from |d|=40 nt to |d|=4 nt. FIG. 16A depicts the mechanism for an allosteric OFF→ON split-terminator switch cgRNA and trigger (depicted for a 10 bp terminator duplex with domain lengths |d|=|d*|=10 nt). For terminator duplexes of length 4, 6, 8, or 10 bp (with domain length |d|=4, 6, 8, 10 nt), FIG. 16B demonstrates that expression of RNA trigger X (PEL+ {4, 6, 8, 10} nt+terminator hairpin) toggles the cgRNA from OFF→ON in human cells, leading to an increase in fluorescence. For terminator duplexes of length 10, 20, 30, 40 bp (with domain length |d]=10, 20, 30, 40 nt), FIG. 16C demonstrates that expression of RNA trigger X (PEL+ {10, 20, 30, 40} nt+terminator hairpin) toggles the cgRNA from OFF→ON in human cells, leading to an increase in fluorescence. Transfection of plasmids expressing inducing dCas9-VPR, Phi-YFP target gene Y, and either: cgRNA+no-trigger control (OFF state), cgRNA+RNA trigger X (ON state). For each set of conditions, 50,000 cells were collected from a single well, which was gated for live cells, single cells, and highly-transfected cells based on the cgRNA plasmid transfection control (miRFP670+), yielding approximately 2% of the cells. Bar graph depicts mean single-cell fluorescence over approximately 1000 cells for one well. FIG. 16D depicts the sequences of cgRNAs and triggers for the studies of FIG. 16B with terminator duplexes of 6, 8, or 10 bp (cgRNA and trigger sequences with 4 bp terminator duplexes are displayed in FIG. 14H). FIG. 16E depicts the sequences of cgRNAs and triggers for the studies of FIG. 16B with terminator duplexes of 10, 20, 30, or 40 bp. Nucleotides that are lower case italic are constrained by the target binding site on the reporter plasmid. Nucleotides shaded gray are constrained by dCas9. Nucleotides that are upper case italic are designed. The plain “C” nucleotide is a cloning artifact. Lower case plain nucleotides are constrained by the hU6 terminator sequence⁴⁶. Bold nucleotides are a protective element (PEL) constrained by an xrRNA sequence derived from Dengue (Dengue 4, NC_002640.1)⁴⁷. Sequences are presented as DNA sequences; the corresponding RNA sequence is the same as the DNA sequence except that “U” replaces “T”).

### Example—Demonstration of Inactivation of a gRNA in Human Cells Via Truncation from the 3′ End

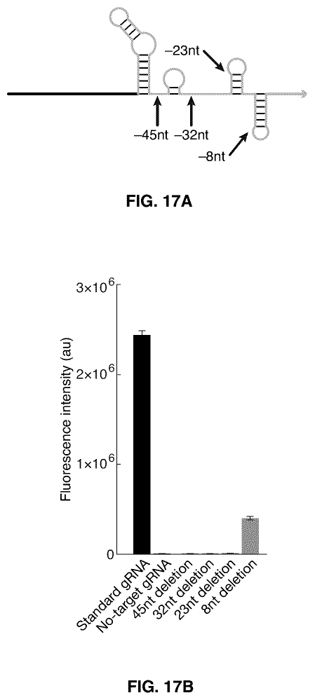

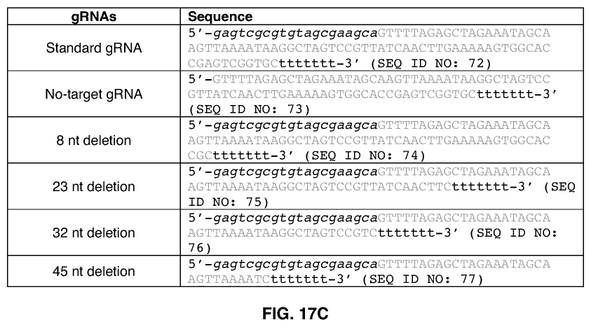

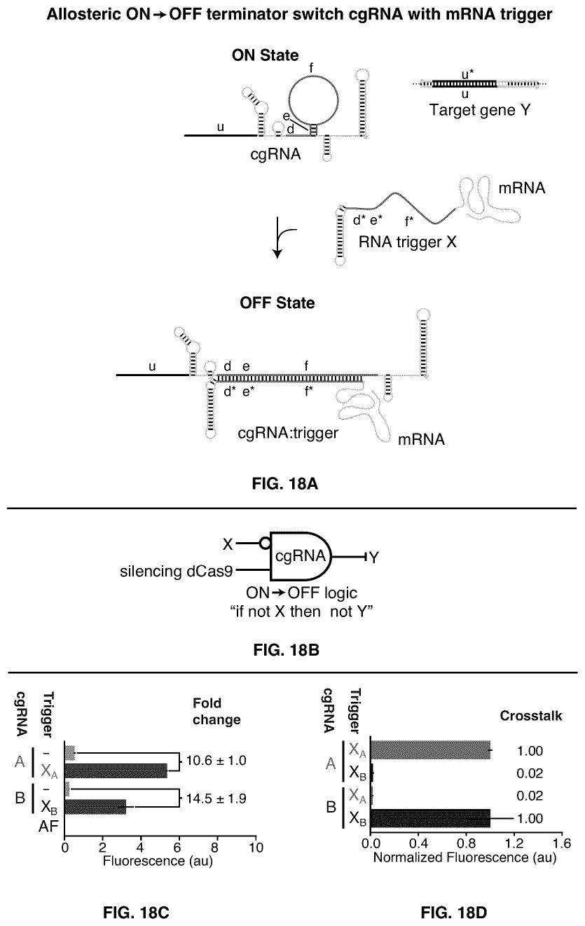

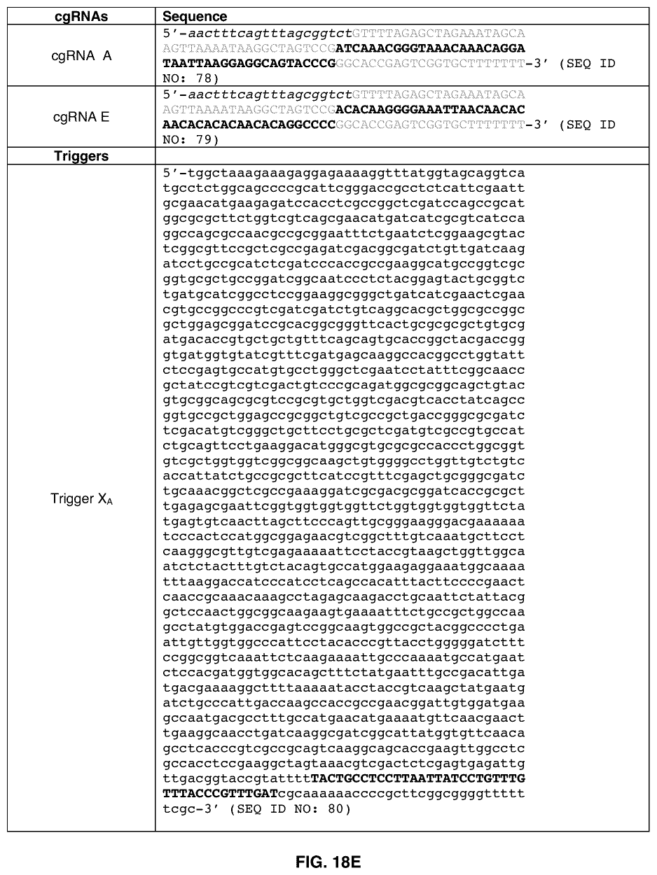

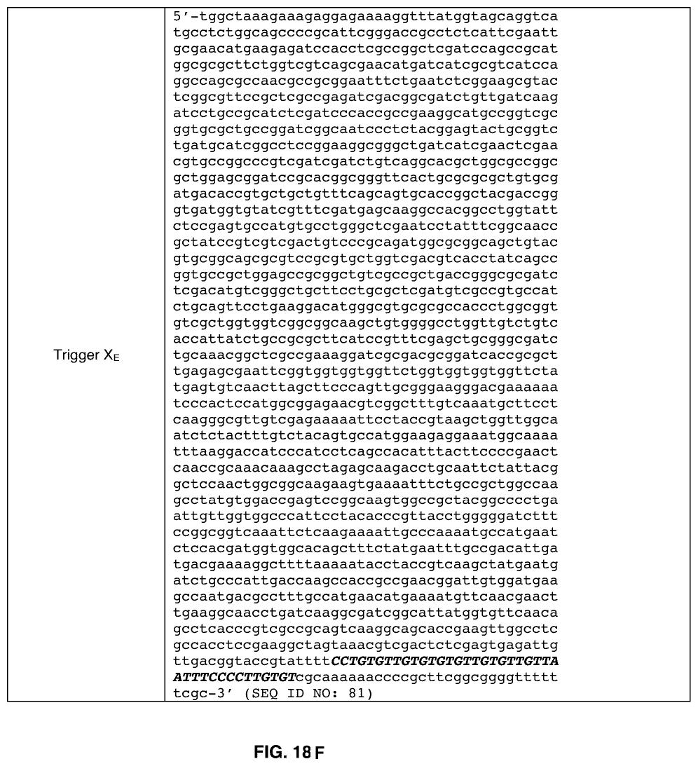

FIG. 17 demonstrates inactivation of a standard gRNA in HEK 293T cells by truncation of the gRNA from the 3′ end, providing the basis for generating a clean OFF state for split-terminator switch cgRNAs. FIG. 17A depicts four truncation locations: 3′ of the Cas9 handle (45 nt deletion), 3′ of the nexus hairpin (32 nt deletion), in the loop of terminator hairpin 1 (23 nt deletion), and in the loop of terminator hairpin 2 (8 nt deletion). FIG. 18C demonstrates that all four truncation locations reduce the activity of the gRNA, with the 45 nt, 32 nt, and 23 nt truncations resulting in no detectable activity. Transfection of plasmids expressing inducing dCas9-VPR, Phi-YFP target gene Y, and either: standard gRNA+no-trigger control (ideal ON state), no-target gRNA that lacks target-binding region+no-trigger control (ideal OFF state), the 5′ portion of a truncated gRNA+no-trigger control. Bar graphs depict mean+estimated standard error of the mean calculated based on the mean single-cell fluorescence over 2114-3085 cells for each of N=3 replicate wells. FIG. 17C depicts the sequences used in FIG. 17B. Nucleotides that are lower case italic are constrained by the target binding site on the reporter plasmid. Nucleotides shaded gray are constrained by dCas9. Lower case plain nucleotides are constrained by the hU⁶ terminator sequence⁴⁶. Sequences are presented as DNA sequences; the corresponding RNA sequence is the same as the DNA sequence except that “U” replaces “T”).

### Example—Demonstration of Allosteric OFF→ON Split-Terminator Switch cgRNAs in Bacteria

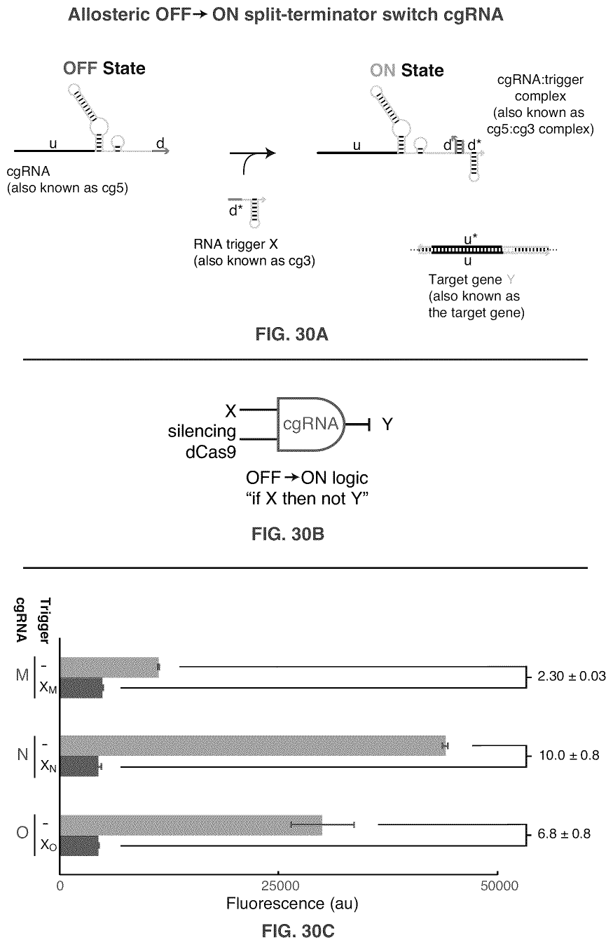

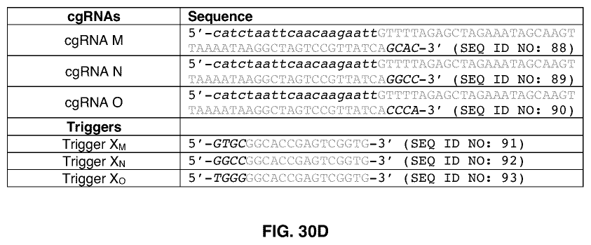

FIG. 30 demonstrates allosteric OFF→ON split-terminator switch cgRNAs performing conditional logic in E. coli. FIG. 30A depicts the mechanism for an allosteric OFF→ON split-terminator switch cgRNA: the constitutively inactive cgRNA is activated by hybridization of RNA trigger X. Rational design of 4 bp terminator duplex (cgRNA domain “d” and trigger domain “d*”). FIG. 30B depicts the conditional logic for an OFF→ON split-terminator switch cgRNA used in conjunction with silencing dCas9: “if X then not Y” (if trigger X is detected, then silence target gene Y). FIG. 30C demonstrates that expression of RNA trigger X (4 nt+terminator hairpin) toggles the activity of the cgRNA from OFF→ON (leading to a decrease in fluorescence) for each of three cgRNAs (M, N, O) with their corresponding cognate triggers (X_{M}, X_{N}, X_{O}). Induced expression (aTc) of silencing dCas9 and constitutive expression of sfGFP target gene Y and either: cgRNA (OFF state), cgRNA+RNA trigger X (ON state). In FIG. 30C, raw fluorescence depicts OFF→ON conditional response to cognate trigger. Fold change=OFF/ON=[no trigger]/[cognate trigger]. Bar graphs depict mean±estimated standard error calculated based on the mean single-cell fluorescence over 20,000 cells for each of N=2 replicate wells (OFF:ON ratio calculated with uncertainty propagation). FIG. 30D depicts the sequences of cgRNAs M, N, O, and the sequences of triggers X_{M}, X_{N}, X_{O}. Nucleotides that are lower case italic are constrained by the target gene. Nucleotides shaded gray are constrained by dCas9. Nucleotides that are upper case italic are designed. Sequences are presented as DNA sequences; the corresponding RNA sequence is the same as the DNA sequence except that “U” replaces “T”).

In E. coli expressing an allosteric OFF→ON split terminator switch (FIG. 30A), silencing dCas9 and a fluorescent protein reporter (sfGFP) as the target gene Y (conditional logic: “if X then Y”; FIG. 30B), the cgRNA exhibits a strong conditional response to expression of RNA trigger X (FIG. 30C). FIG. 30C displays raw fluorescence data for a library of three orthogonal OFF→ON split terminator switch cgRNAs with and without their cognate triggers. For this library of three cgRNA/trigger pairs, the median fold-change is approximately 7× for the OFF→ON conditional response to expression of the cognate trigger (FIG. 30C).

cgRNA/trigger pairs were designed using NUPACK.^{40,41} cgRNA/trigger plasmids were transformed into a modified E. coli MG1655 strain expressing genomically incorporated mRFP and sfGFP4.⁵ Strains were grown overnight in EZ-RDM (Teknova), then diluted 500 fold with fresh medium containing aTc for induction of silencing dCas9 expression. Induced cells were grown for 12 h, with end-point fluorescence measured via flow cytometry.

### Example—Demonstration of Allosteric ON→OFF Split-Terminator Switch cgRNAs in Human Cells

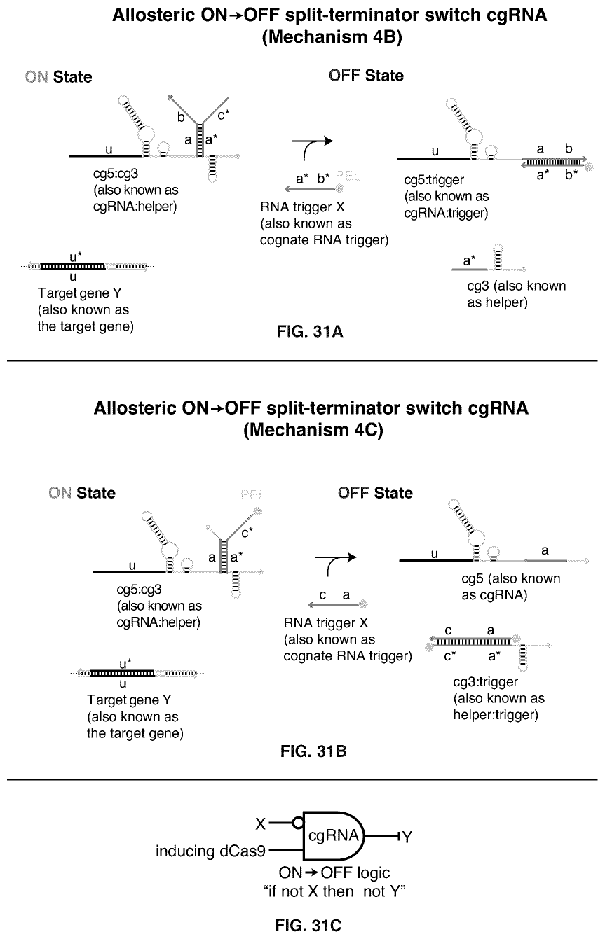

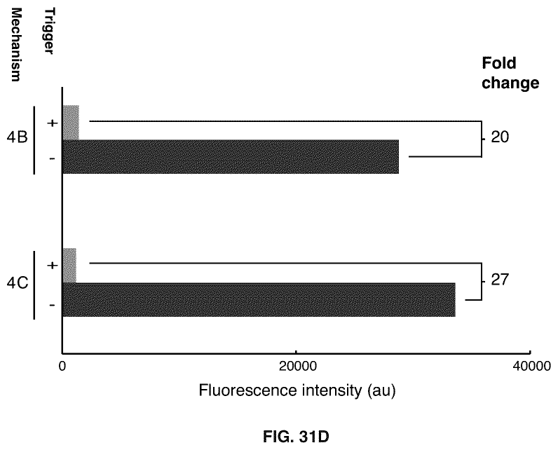

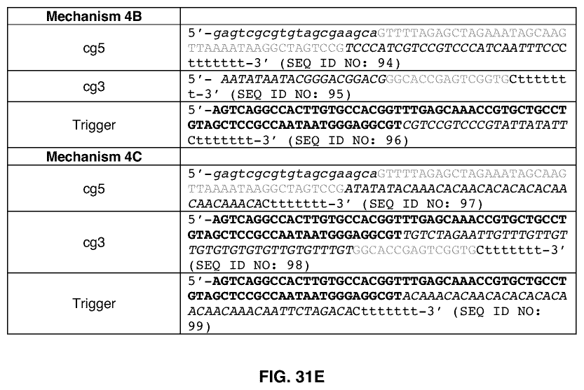

FIG. 31 demonstrates allosteric ON→OFF split-terminator switch cgRNAs performing conditional logic in HEK 293T cells. FIG. 31A depicts the mechanism for an allosteric ON→OFF split-terminator switch cgRNAs (Mechanism 4B): the constitutively active cgRNA (comprising 5′ fragment cg5 [also known as cgRNA] and 3′ fragment cg3 [also known as helper]) is inactivated by hybridization of RNA trigger X (also known as the cognate RNA trigger) to cg5, displacing cg3 from cg5 to inactivate the cgRNA. FIG. 31B depicts the mechanism for an allosteric ON→OFF split-terminator switch cgRNAs (Mechanism 4C): the constitutively active cgRNA (comprising 5′ fragment cg5 [also known as cgRNA] and 3′ fragment cg3 [also known as helper]) is inactivated by hybridization of RNA trigger X (also known as the cognate RNA trigger) to cg3, displacing cg5 from cg3 to inactivate the cgRNA. Rational sequence design of domains “a”, “b”, “c”, “a*”, “b*”, and “c*”. FIG. 31C depicts the conditional logic for an ON→OFF split-terminator switch cgRNA used in conjunction with inducing dCas9: “if not X then Y” (if trigger X is not detected, induce target gene Y).

FIG. 31D demonstrates that expression of RNA trigger X (PEL+20 nt unstructured+hU6 terminator for Mechanism 4B; PEL+40 nt unstructured+hU6 terminator for Mechanism 4C) toggles the cgRNA from ON→OFF, leading to a decrease in fluorescence. Transfection of plasmids expressing inducing dCas9-VPR, Phi-YFP target gene Y, and either: cgRNA+no-trigger control (ON state), or cgRNA+RNA trigger X (OFF state). The no-trigger control uses a random pool of triggers to provide a sequence-generic approximation of the metabolic load of trigger expression. Background subtracted fluorescence by no-target gRNA that lacks target-binding region depicts ON→OFF conditional response to cognate trigger. Fold change=ON/OFF. Bar graphs depict the mean single-cell fluorescence over 1989-2305 cells for N=1 well. FIG. 31E depicts the sequences of cg5, cg3, and trigger for Mechanism 4B and 4C. Nucleotides that are lower case italic are constrained by the target binding site on the reporter plasmid. Nucleotides shaded gray are constrained by dCas9. Nucleotides that are upper case italic are designed. The plain “C” nucleotide is a cloning artifact. Lower case plain nucleotides are constrained by the hU6 terminator sequence⁴⁶. Bold nucleotides are a protective element (PEL) constrained by an xrRNA sequence derived from Dengue (Dengue 4, NC_002640.1)⁴⁷. Sequences are presented as DNA sequences; the corresponding RNA sequence is the same as the DNA sequence except that “U” replaces “T”).

In HEK 293T cells expressing an allosteric ON→OFF split-terminator switch cgRNA (Mechanism 4B in FIG. 31A; Mechanism 4C in FIG. 31B), inducing dCas9-VPR as the protein effector,⁴⁸ and a fluorescent protein reporter (Phi-YFP)^{49,50} as the target gene Y (conditional logic: “if not X then Y”; FIG. 31C), the cgRNA (comprising fragments cg5 and cg3) exhibits a strong conditional response to expression of the RNA trigger X (FIG. 31D).

To optimize fold-change, the goal is to maximize the ON→OFF or OFF→ON conditional response ratio with/without the cognate RNA trigger (higher is better). The cgRNA/trigger pairs were designed using NUPACK^{40,41}. A cg5 and cg3 expression plasmid and a trigger expression plasmid were co-transfected with a plasmid expressing an inducing dCas9-VPR fusion⁴⁸ and a reporter plasmid containing a gRNA binding site upstream of a minimal CMV promoter for Phi-YFP expression.^{51,52} The four plasmids were transiently transfected into HEK 293T cells with Lipofectamine 2000 and grown for 24 h, with end-point fluorescence measured via flow cytometry. Data analysis was performed on cells expressing high levels of both cgRNA and trigger fluorescent protein transfection controls.

### Example—Demonstration of Allosteric ON→OFF Split-Terminator Switch cgRNAs in Bacteria

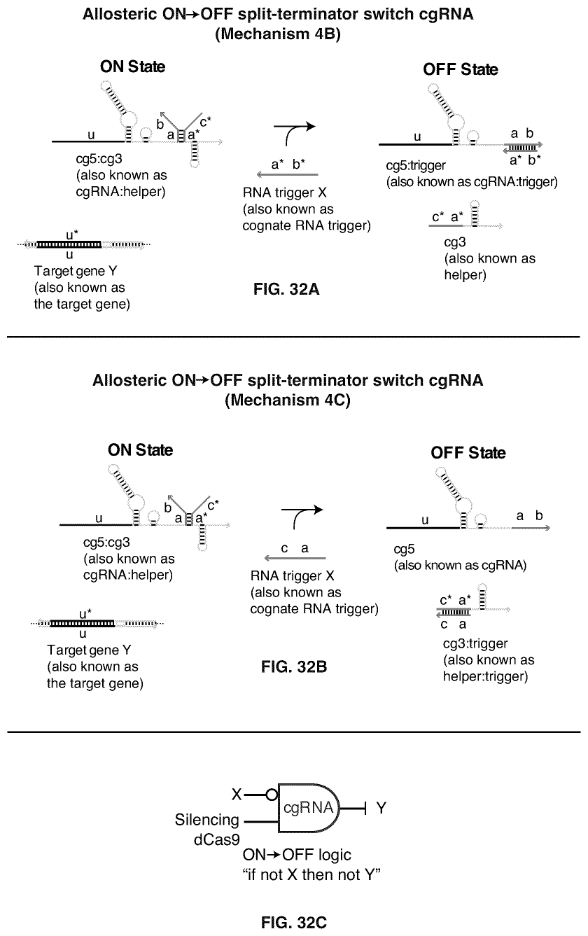

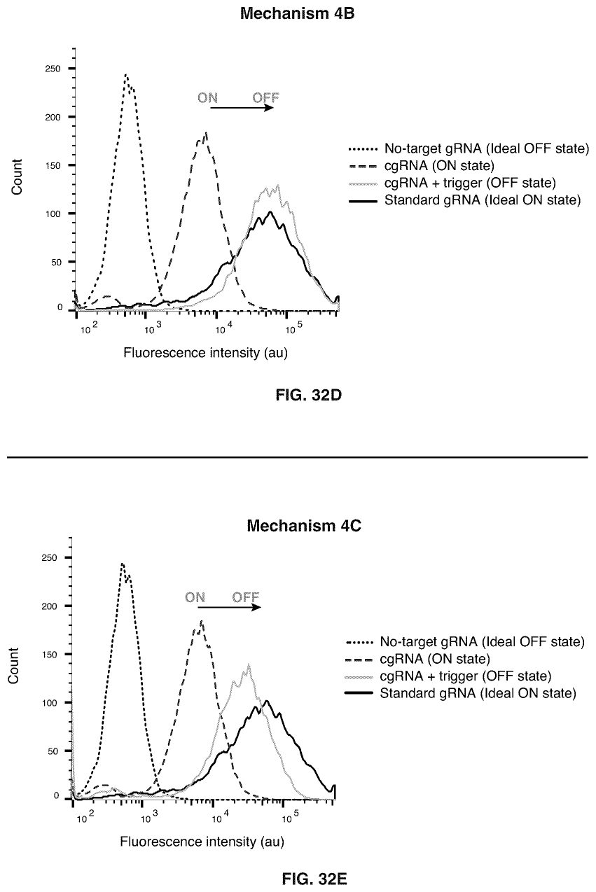

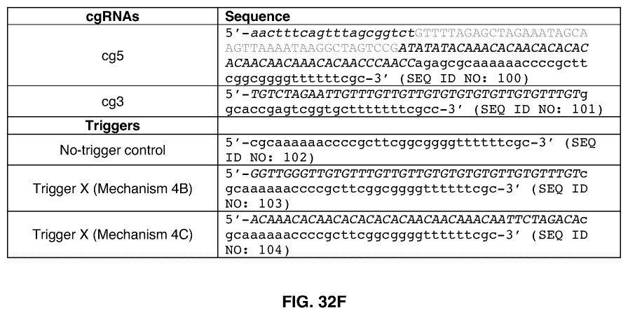

FIG. 32 demonstrates allosteric ON→OFF split-terminator switch cgRNAs performing conditional logic in E. coli. FIG. 32A depicts the mechanism for an allosteric ON→OFF split-terminator switch cgRNAs (Mechanism 4B): the constitutively active cgRNA (comprising 5′ fragment cg5 [also known as cgRNA] and 3′ fragment cg3 [also known as helper]) is inactivated by hybridization of RNA trigger X (also known as the cognate RNA trigger) to cg5, displacing cg3 from cg5 to inactivate the cgRNA. FIG. 32B depicts the mechanism for an allosteric ON→OFF split-terminator switch cgRNAs (Mechanism 4C): the constitutively active cgRNA (comprising 5′ fragment cg5 [also known as cgRNA] and 3′ fragment cg3 [also known as helper]) is inactivated by hybridization of RNA trigger X (also known as the cognate RNA trigger) to cg3, displacing cg5 from cg3 to inactivate the cgRNA. FIG. 32C depicts the conditional logic for an ON→OFF split-terminator switch cgRNA used in conjunction with silencing dCas9: “if not X then not Y” (if trigger X is not detected, then silence target gene Y). FIGS. 32D (Mechanism 4B) and 32E (Mechanism 4C) demonstrate that expression of RNA trigger X (40 nt unstructured+synthetic terminator hairpin) toggles the cgRNA from ON→OFF, leading to an increase in single-cell fluorescence intensities via flow cytometry. Induced expression (aTc) of silencing dCas9 and constitutive expression of mRFP target gene Y and either: standard gRNA+no-trigger control (ideal ON state), cgRNA+no-trigger control (ON state), cgRNA+RNA trigger X (OFF state; trigger expression is Salicylate-induced), no-target gRNA that lacks target-binding region+no-trigger control (ideal OFF state). FIG. 32F depicts the sequences of cg5, cg3, trigger X (Mechanism 4B), trigger X (Mechanism 4C), and the no-trigger control. Nucleotides that are lower case italic are constrained by the target gene. Nucleotides shaded gray are constrained by dCas9. Nucleotides that are upper case italic are designed. Sequences are presented as DNA sequences; the corresponding RNA sequence is the same as the DNA sequence except that “U” replaces “T”).

Orthogonal cgRNA/trigger pairs were designed using NUPACK.^{40,41} cgRNA/trigger plasmids were transformed into E. coli.⁵ Strains were grown overnight in EZ-RDM (Teknova), then diluted and seeded into the assay plate. Cells were grown in fresh medium containing aTc, IPTG, and Salicylate for induction of silencing dCas9 expression, cgRNA, and trigger RNA. Induced cells were grown for 12 h, with end-point fluorescence measured via flow cytometry.

### Example—Demonstration of Allosteric OFF→ON 5′- and 3′-Inhibited Split-Terminator Switch cgRNAs in Bacteria

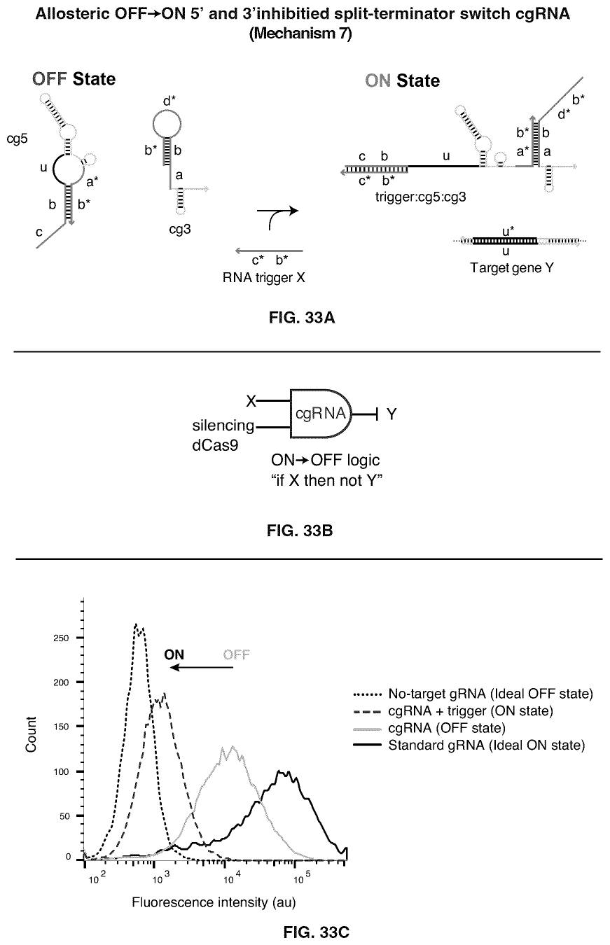

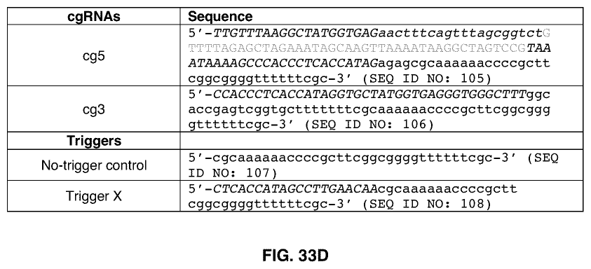

FIG. 33 demonstrates allosteric OFF→ON 5′- and 3′-inhibited split-terminator switch cgRNAs (Mechanism 7) performing conditional logic in E. coli. FIG. 33A depicts the mechanism for an allosteric OFF→ON 5′- and 3′-inhibited split-terminator switch cgRNA (Mechanism 7): the constitutively inactive cgRNA comprising 5′ fragment cg5 and 3′ fragment cg3 is activated by hybridization of RNA trigger X to cg5, allowing for cg5 to bind to cg3 and activate the cgRNA. FIG. 33B depicts the conditional logic for an OFF→ON 5′- and 3′-inhibited split-terminator switch cgRNA used in conjunction with silencing dCas9: “if X then not Y” (if trigger X is detected, then silence target gene Y). FIG. 33C demonstrates that expression of RNA trigger X (20 nt unstructured+synthetic terminator hairpin) toggles the cgRNA from OFF→ON, leading to a decrease in single-cell fluorescence intensities via flow cytometry. Induced expression (aTc) of silencing dCas9 and constitutive expression of mRFP target gene Y and either: standard gRNA+no-trigger control (ideal ON state), cgRNA+no-trigger control (ON state), cgRNA+RNA trigger X (OFF state; trigger expression is Salicylate-induced), no-target gRNA that lacks target-binding region+no-trigger control (ideal OFF state). FIG. 32F depicts the sequences of cg5, cg3, trigger X (Mechanism 4B), trigger X (Mechanism 4C), and the no-trigger control. Nucleotides that are lower case italic are constrained by the target gene. Nucleotides shaded gray are constrained by dCas9. Nucleotides that are upper case italic are designed. Sequences are presented as DNA sequences; the corresponding RNA sequence is the same as the DNA sequence except that “U” replaces “T”).

Orthogonal cgRNA/trigger pairs were designed using NUPACK.40,41 cgRNA/trigger plasmids were transformed into E. coli.5 Strains were grown overnight in EZ-RDM (Teknova), then diluted and seeded into the assay plate. Cells were grown in fresh medium containing aTc, IPTG, and Salicylate for induction of silencing dCas9 expression, cgRNA, and trigger RNA. Induced cells were grown for 12 h, with end-point fluorescence measured via flow cytometry.

### Example—Demonstration of Allosteric cgRNAs Performing Conditional Logic in Response to mRNA Triggers

FIG. 18 demonstrates allosteric cgRNAs performing conditional logic in response to detection of long RNA triggers in E. coli. FIG. 18A depicts the mechanism for an allosteric ON→OFF terminator switch cgRNA: the constitutively active cgRNA is inactivated by hybridization of RNA trigger X (which is a full-length mRNA trigger molecule containing subsequence “f*-e*-d*” that serves as the trigger domain). FIG. 18B depicts the conditional logic for an ON→OFF terminator switch cgRNA used in conjunction with silencing dCas9: “if not X then not Y” (if trigger X is not detected, then silence target gene Y). FIG. 18C demonstrates that expression of RNA trigger X (full-length mRNA) toggles the cgRNA from ON→OFF, leading to an increase in fluorescence. 18C and 18D demonstrate programmable conditional regulation using 2 orthogonal cgRNAs (A, B) and their corresponding cognate triggers (X_{A}, X_{B}). Induced expression (aTc) of silencing dCas9 and constitutive expression of mRFP target gene Y and either: cgRNA (ON state), cgRNA+RNA trigger X (OFF state; trigger expression is IPTG-induced). Autofluorescence (AF): cells with no mRFP. In FIG. 18C, raw fluorescence depicts ON→OFF conditional response to cognate trigger. Fold change =OFF/ON=[cognate trigger−AF]/[no trigger−AF]). In FIG. 18D, normalized fluorescence depicts orthogonality between non-cognate cgRNA/trigger pairs. Crosstalk=[non-cognate trigger-no trigger]/[cognate trigger-no trigger]). Bar graphs depict mean±estimated standard error calculated based on the mean single-cell fluorescence over 20,000 cells for each of N=3 replicate wells (OFF:ON ratio and crosstalk calculated with uncertainty propagation). FIG. 18E depicts the sequences of cgRNAs A, B and the sequences of mRNA trigger molecules X_{A}, X_{B}. Nucleotides that are lower case italic are constrained by the target gene. Nucleotides shaded gray are constrained by dCas9. Nucleotides in a cgRNA that are upper case bold are complementary to the trigger domain of the corresponding trigger molecule. Nucleotides in an mRNA trigger molecule are lower case except for the trigger domain which is upper case bold. Sequences are presented as DNA sequences; the corresponding RNA sequence is the same as the DNA sequence except that “U” replaces “T”).

### Example—Computational Sequence Design of Orthogonal cgRNA/Trigger Pairs

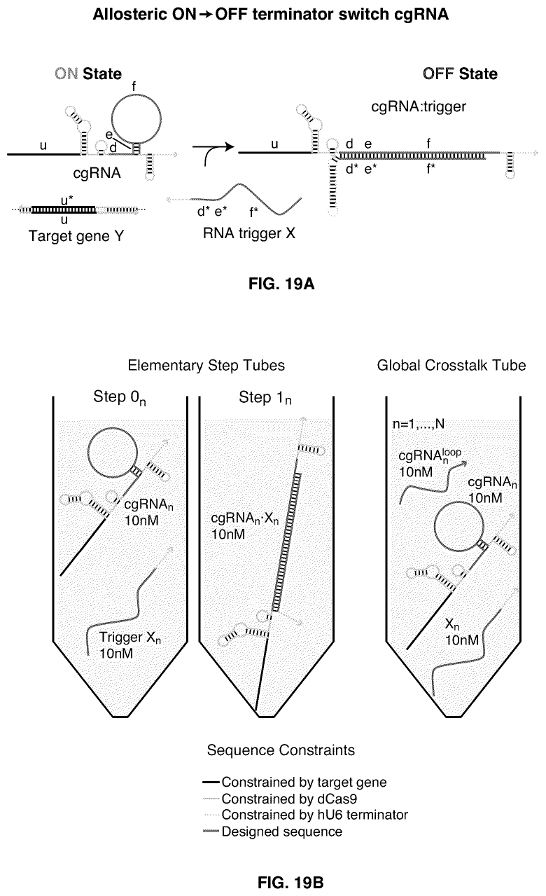

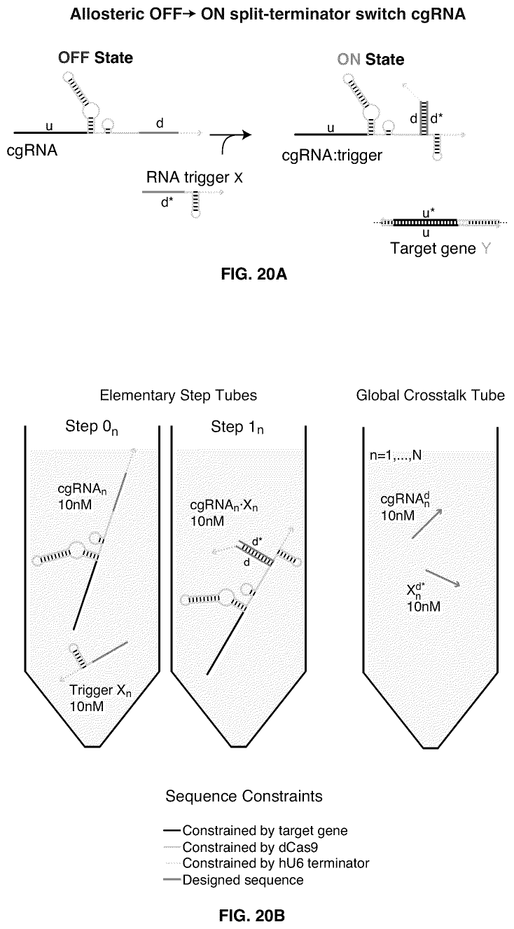

FIG. 19B displays target test tubes for the design of N orthogonal cgRNA/trigger pairs for the ON→OFF terminator switch mechanism (FIG. 19A). FIG. 20B displays target test tubes for the design of N orthogonal cgRNA/trigger pairs for the OFF→ON split-terminator switch mechanism (FIG. 20A). To design N orthogonal cgRNA/trigger pairs (systems), there are two elementary step tubes for each system (n=1, . . . , N): 1) A Reactants tube (Step On) containing cgRNAn and the corresponding trigger Xn; 2) A Products tube (Step In) containing the complex cgRNAn:Xn. Each target test tube contains a set of desired “on-target” complexes (each with the depicted target secondary structure and target concentration (10 nM in this example)) corresponding to the on-pathway hybridization products for a given step and a set of undesired “off-target” complexes (in this example, all complexes of up to 2 strands, each with a target concentration of 0 nM; not depicted) corresponding to on-pathway reactants and off-pathway hybridization crosstalk for a given step; these elementary step tubes are designed for full conversion of cognate reactants into cognate products and against local hybridization crosstalk between these same reactants. To design N orthogonal systems, there is a single global crosstalk tube containing a set of on-targets and off-targets for each system. The global crosstalk tube contains the depicted on-target complexes corresponding to reactive species generated during Steps 0 and 1 (each with the depicted target secondary structure and target concentration (10 nM in this example) as well as off-target complexes (for this example, all complexes of up to 2 strands, each with a target concentration of 0 nM; not depicted) corresponding to off-pathway interactions between these reactive species. Crucially, the global crosstalk tube ensemble omits the cognate products that the reactive species are intended to form (they appear as neither on-targets nor off-targets). Hence, all reactive species in the global crosstalk tube are forced to either perform no reaction (remaining as desired on-targets) or undergo a crosstalk reaction (forming undesired off-targets), providing the basis for minimization of global crosstalk during sequence optimization. To design a library of N orthogonal cgRNA/trigger pairs, all N cgRNAs have the same on-target structure, and all N triggers have the same on-target structure; within a library, the only difference between cgRNA/trigger pairs is the designed sequence; there are a total of 2N elementary step tubes plus 1 global crosstalk tube. For this example, sequence design was performed subject to complementarity constraints inherent to the reaction pathway (FIGS. 19A and 20A; domain “d″complementary to “d*”, etc.), as well as to biological sequence constraints imposed by the the silencing target Y, the protein effector (dCas9), and the terminator; see the constraint shading in FIGS. 19B and 20B. The sequence was optimized by reducing the ensemble defect quantifying the average fraction of incorrectly paired nucleotides over the multi-tube ensemble.^{41, 53, 54} Within the ensemble defect, defect weights were applied to prioritize design effort.⁴¹ Optimization of the ensemble defect implemented both a positive design paradigm (explicitly designing for on-pathway elementary steps) and a negative-design paradigm (explicitly designing against off-pathway crosstalk).⁴¹

### Example—Demonstration of Allosteric ON→OFF and OFF→ON cgRNAs Functioning in Multi-Cellular Organisms

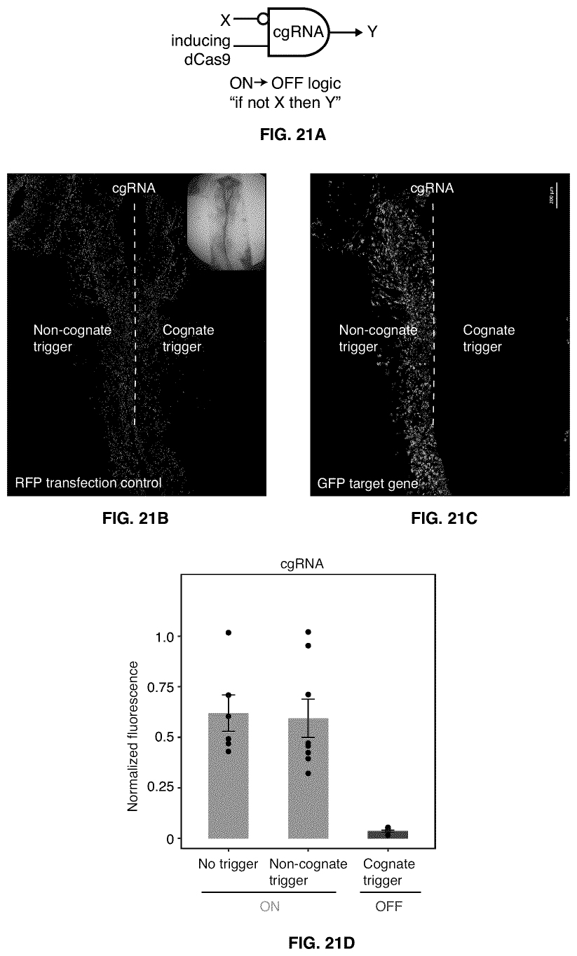

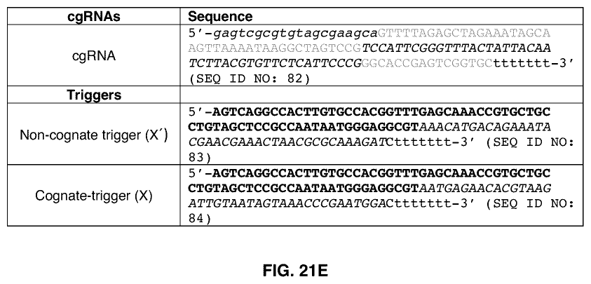

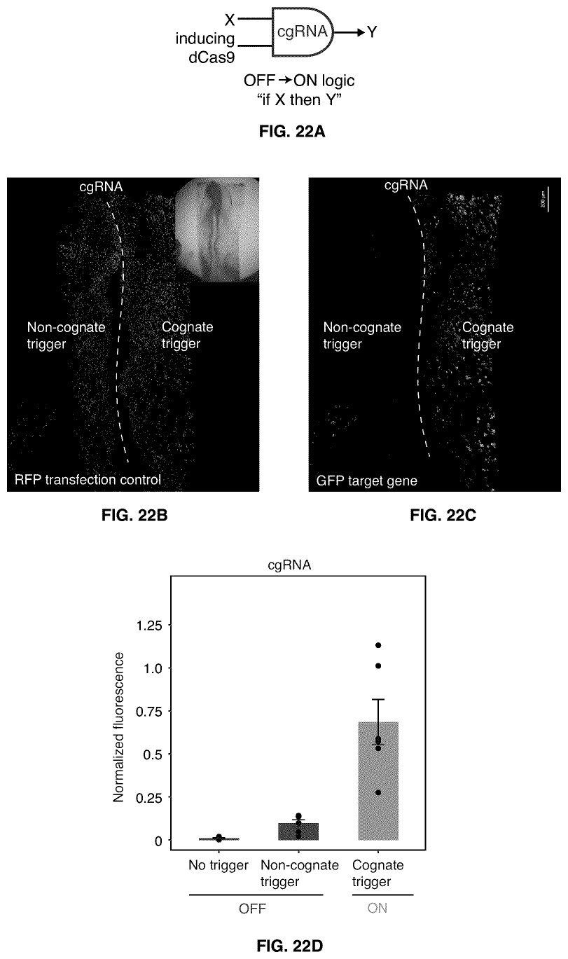

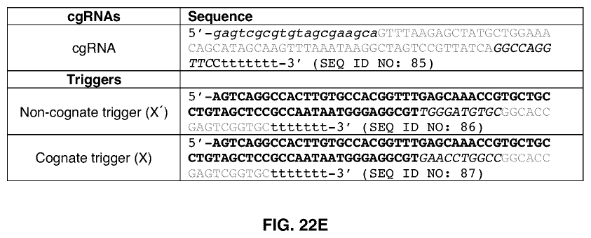

FIGS. 21 and 22 demonstrate allosteric cgRNAs functioning in developing chicken embryos.

FIG. 21 demonstrates an allosteric ON→OFF terminator switch cgRNA performing conditional logic in a chicken embryo (mechanism of FIG. 13A). FIG. 21A depicts the conditional logic for an ON→OFF terminator switch cgRNA used in conjunction with inducing dCas9: “if not X then Y” (if trigger X is not detected, induce target gene Y). For the experimental demonstration, five plasmids are electroporated into the left side and right side (independently for each side) of a developing chicken embryo (plasmids transfected into both sides: RFP transfection control, inducing dCas9, cgRNA, d2eGFP as the target gene Y; plasmid transfected into the left side only: non-cognate trigger X′, plasmid transfected into the right side only: cognate trigger X). FIG. 21B displays RFP fluorescence for the transfection control on both sides of the embryo, demonstrating transfection into both sides of the embryo. FIG. 21C displays GFP fluorescence of the target gene Y, exhibiting high fluorescence on the left side of the embryo (ON state; non-cognate trigger) and low fluorescence on the right side of the embryo (OFF state; cognate trigger). The cognate trigger toggles the cgRNA from the ON state to the OFF state, leading to a large reduction in GFP fluorescence, corresponding to reduced expression of the target gene Y. FIG. 21D displays normalized fluorescence for representative regions in replicate embryos (one dot per embryo) for experiments with transfection of either: no trigger (ON state), non-cognate trigger (ON state), or cognate trigger (OFF state). Error bars represent standard error of the mean over replicate embryos. FIG. 21E depicts the sequences of cgRNAs, the cognate trigger X, and the non-cognate trigger X′. Nucleotides that are lower case italic are constrained by the target binding site on the reporter plasmid. Nucleotides shaded gray are constrained by dCas9. Nucleotides that are upper case italic are designed. The plain “C” nucleotide is a cloning artifact. Lower case plain nucleotides are constrained by the hU6 terminator sequence⁴⁶. Bold nucleotides are a protective element (PEL) constrained by an xrRNA sequence derived from Dengue (Dengue 4, NC_002640.1)⁴⁷. Sequences are presented as DNA sequences; the corresponding RNA sequence is the same as the DNA sequence except that “U” replaces “T”).

FIG. 22 demonstrates an allosteric OFF→ON split-terminator switch cgRNA performing conditional logic in a chicken embryo (mechanism of FIG. 15A). FIG. 22A depicts the conditional logic for an OFF→ON split-terminator switch cgRNA used in conjunction with inducing dCas9: “if X then Y” (if trigger X is detected, induce target gene Y). For the experimental demonstration, five plasmids are electroporated into the left side and right side (independently for each side) of a developing chicken embryo (plasmids transfected into both sides: RFP transfection control, inducing dCas9, cgRNA, d2eGFP as the target gene Y; plasmid transfected into the left side only: non-cognate trigger X′, plasmid transfected into the right side only: cognate trigger X). FIG. 22B displays RFP fluorescence for the transfection control on both sides of the embryo, demonstrating transfection into both sides of the embryo. FIG. 22C displays GFP fluorescence of the target gene Y, exhibiting low fluorescence on the left side of the embryo (OFF state; non-cognate trigger) and high fluorescence on the right side of the embryo (ON state; cognate trigger). The cognate trigger toggles the cgRNA from the OFF to the ON state, leading to a large increase in GFP fluorescence, corresponding to increased expression of the target gene Y. FIG. 21D displays normalized fluorescence for representative regions in replicate embryos (one dot per embryo) for experiments with transfection of either: no trigger (OFF state), non-cognate trigger (OFF state), or cognate trigger (ON state). Error bars represent standard error of the mean over replicate embryos. FIG. 22E depicts the sequences of cgRNAs, the cognate trigger X, and the non-cognate trigger X′. Nucleotides that are lower case italic are constrained by the target binding site on the reporter plasmid. Nucleotides shaded gray are constrained by dCas9. Nucleotides that are upper case italic are designed. The plain “C” nucleotide is a cloning artifact. Lower case plain nucleotides are constrained by the hU6 terminator sequence⁴⁶. Bold nucleotides are a protective element (PEL) constrained by an xrRNA sequence derived from Dengue (Dengue 4, NC_002640.1).⁴⁷ Sequences are presented as DNA sequences; the corresponding RNA sequence is the same as the DNA sequence except that “U” replaces “T”).

### REFERENCES

ADDIN ZOTERO_BIBL {“uncited”: [ ], “omitted”: [ ], “custom”: [ ]} CSL_BIBLIOGRAPHY (1) Cong, L.; Ran, F. A.; Cox, D.; Lin, S.; Barretto, R.; Habib, N.; Hsu, P. D.; Wu, X.; Jiang, W.; Marraffini, L. A.; Zhang, F. Multiplex Genome Engineering Using CRISPR/Cas Systems. Science 2013, 339 (6121), 819-823. https://doi.org/10.1126/science.1231143.
(2) Mali, P.; Yang, L.; Esvelt, K. M.; Aach, J.; Guell, M.; DiCarlo, J. E.; Norville, J. E.; Church, G. M. RNA-Guided Human Genome Engineering via Cas9. Science 2013, 339 (6121), 823.
(3) Zetsche, B.; Heidenreich, M.; Mohanraju, P.; Fedorova, I.; Kneppers, J.; DeGennaro, E. M.; Winblad, N.; Choudhury, S. R.; Abudayyeh, O. O.; Gootenberg, J. S.; Wu, W. Y.; Scott, D. A.; Severinov, K.; van der Oost, J.; Zhang, F. Multiplex Gene Editing by CRISPR-Cpf1 Using a Single CrRNA Array. Nat Biotechnol 2016, 35, 31-34.
(4) Knott, G. J.; Doudna, J. A. CRISPR-Cas Guides the Future of Genetic Engineering. Science 2018, 361 (6405), 866-869.
(5) Qi, L. S.; Larson, M. H.; Gilbert, L. A.; Doudna, J. A.; Weissman, J. S.; Arkin, A. P.; Lim, W. A. Repurposing CRISPR as an RNA-Guided Platform for Sequence-Specific Control of Gene Expression. Cell 2013, 152 (5), 1173-1183. https://doi.org/10.1016/j.cell.2013.02.022.
(6) Larson, M. H.; Gilbert, L. A.; Wang, X.; Lim, W. A.; Weissman, J. S.; Qi, L. S. CRISPR Interference (CRISPRi) for Sequence-Specific Control of Gene Expression. Nat Protoc 2013, 8, 2180-2196.
(7) Gilbert, L. A.; Larson, M. H.; Morsut, L.; Liu, Z.; Brar, G. A.; Torres, S. E.; Stern-Ginossar, N.; Brandman, O.; Whitehead, E. H.; Doudna, J. A.; Lim, W. A.; Weissman, J. S.; Qi, L. S. CRISPR-Mediated Modular RNA-Guided Regulation of Transcription in Eukaryotes. Cell 2013, 154 (2), 442-451.
(8) Anzalone, A. V.; Randolph, P. B.; Davis, J. R.; Sousa, A. A.; Koblan, L. W.; Levy, J. M.; Chen, P. J.; Wilson, C.; Newby, G. A.; Raguram, A.; Liu, D. R. Search-and-Replace Genome Editing without Double-Strand Breaks or Donor DNA. Nature 2019, 576 (7785), 149-157. https://doi.org/10.1038/s41586-019-1711-4.
(9) Liu, X. S.; Wu, H.; Krzisch, M.; Wu, X.; Graef, J.; Muffat, J.; Hnisz, D.; Li, C. H.; Yuan, B.; Xu, C.; Li, Y.; Vershkov, D.; Cacace, A.; Young, R. A.; Jaenisch, R. Rescue of Fragile X Syndrome Neurons by DNA Methylation Editing of the FMR1 Gene. Cell 2018, 172 (5), 979-992.e6. https://doi.org/10.1016/j.cell.2018.01.012.
(10) Myers, S. A.; Wright, J.; Peckner, R.; Kalish, B. T.; Zhang, F.; Carr, S. A. Discovery of Proteins Associated with a Predefined Genomic Locus via DCas9-APEX-Mediated Proximity Labeling. Nat. Methods 2018, 15 (6), 437-439. https://doi.org/10.1038/s41592-018-0007-1.
(11) Morgan, S. L.; Mariano, N. C.; Bermudez, A.; Arruda, N. L.; Wu, F.; Luo, Y.; Shankar, G.; Jia, L.; Chen, H.; Hu, J.-F.; Hoffman, A. R.; Huang, C.-C.; Pitteri, S. J.; Wang, K. C. Manipulation of Nuclear Architecture through CRISPR-Mediated Chromosomal Looping. Nat. Commun. 2017, 8 (1), 15993. https://doi.org/10.1038/ncomms15993.
(12) Ma, H.; Tu, L.-C.; Naseri, A.; Huisman, M.; Zhang, S.; Grunwald, D.; Pederson, T. Multiplexed Labeling of Genomic Loci with DCas9 and Engineered SgRNAs Using CRISPRainbow. Nat. Biotechnol. 2016, 34 (5), 528-530. https://doi.org/10.1038/nbt.3526.
(13) Mückl, A.; Schwarz-Schilling, M.; Fischer, K.; Simmel, F. C. Filamentation and Restoration of Normal Growth in Escherichia Coli Using a Combined CRISPRi SgRNA/Antisense RNA Approach. PLoS One 2018, 13 (9), e0198058.
(14) Aubrey, B. J.; Kelly, G. L.; Kueh, A. J.; Brennan, M. S.; O'Connor, L.; Milla, L.; Wilcox, S.; Tai, L.; Strasser, A.; Herold, M. J. An Inducible Lentiviral Guide RNA Platform Enables the Identification of Tumor-Essential Genes and Tumor-Promoting Mutations in Vivo. Cell Rep. 2015, 10 (8), 1422-1432.
(15) Bertero, A.; Pawlowski, M.; Ortmann, D.; Snijders, K.; Yiangou, L.; Cardoso de Brito, M.; Brown, S.; Bernard, W. G.; Cooper, J. D.; Giacomelli, E.; Gambardella, L.; Hannan, N. R. F.; Iyer, D.; Sampaziotis, F.; Serrano, F.; Zonneveld, M. C. F.; Sinha, S.; Kotter, M.; Vallier, L. Optimized Inducible ShRNA and CRISPR/Cas9 Platforms for in Vitro Studies of Human Development Using HPSCs. Development 2016, 143 (23), 4405.
(16) Chen, T.; Gao, D.; Zhang, R.; Zeng, G.; Yan, H.; Lim, E.; Liang, F. Chemically Controlled Epigenome Editing through an Inducible DCas9 System. J. Am. Chem. Soc. 2017, 139 (33), 11337-11340. https://doi.org/10.1021/jacs.7b06555.
(17) Moroz-Omori, E. V.; Satyapertiwi, D.; Ramel, M.-C.; Høgset, H.; Sunyovszki, I. K.; Liu, Z.; Wojciechowski, J. P.; Zhang, Y.; Grigsby, C. L.; Brito, L.; Bugeon, L.; Dallman, M. J.; Stevens, M. M. Photoswitchable GRNAs for Spatiotemporally Controlled CRISPR-Cas-Based Genomic Regulation. ACS Cent. Sci. 2020, 6 (5), 695-703. https://doi.org/10.1021/acscentsci.9b01093.
(18) Liu, Y.; Zou, R. S.; He, S.; Nihongaki, Y.; Li, X.; Razavi, S.; Wu, B.; Ha, T. Very Fast CRISPR on Demand. Science 2020, 368 (6496), 1265-1269.
(19) Jain, P. K.; Ramanan, V.; Schepers, A. G.; Dalvie, N. S.; Panda, A.; Fleming, H. E.; Bhatia, S. N. Development of Light-Activated CRISPR Using Guide RNAs with Photocleavable Protectors. Angew. Chem. Int. Ed. 2016, 55 (40), 12440-12444.
(20) Nihongaki, Y.; Otabe, T.; Sato, M. Emerging Approaches for Spatiotemporal Control of Targeted Genome with Inducible CRISPR-Cas9. Anal Chem 2018, 90 (1), 429-439.
(21) Shen, Z.; Zhang, X.; Chai, Y.; Zhu, Z.; Yi, P.; Feng, G.; Li, W.; Ou, G. Conditional Knockouts Generated by Engineered CRISPR-Cas9 Endonuclease Reveal the Roles of Coronin in C. Elegans Neural Development. Dev. Cell 2014, 30 (5), 625-636.
(22) Ablain, J.; Durand, E. M.; Yang, S.; Zhou, Y.; Zon, L. I. A CRISPR/Cas9 Vector System for Tissue-Specific Gene Disruption in Zebrafish. Dev. Cell 2015, 32 (6), 756-764. https://doi.org/10.1016/j.devcel.2015.01.032.
(23) Hirosawa, M.; Fujita, Y.; Parr, C. J. C.; Hayashi, K.; Kashida, S.; Hotta, A.; Woltjen, K.; Saito, H. Cell-Type-Specific Genome Editing with a MicroRNA-Responsive CRISPR-Cas9 Switch. Nucleic Acids Res 2017, 45 (13), e118.
(24) Briner, A. E.; Donohoue, P. D.; Gomaa, A. A.; Selle, K.; Slorach, E. M.; Nye, C. H.; Haurwitz, R. E.; Beisel, C. L.; May, A. P.; Barrangou, R. Guide RNA Functional Modules Direct Cas9 Activity and Orthogonality. Mol Cell 2014, 56 (2), 333-339. https://doi.org/10.1016/j.molcel.2014.09.019.
(25) Nowak, C. M.; Lawson, S.; Zerez, M.; Bleris, L. Guide RNA Engineering for Versatile Cas9 Functionality. Nucleic Acids Res 2016, 44 (20), 9555-9564.
(26) Hanewich-Hollatz, M. H.; Chen, Z.; Hochrein, L. M.; Huang, J.; Pierce, N. A. Conditional Guide RNAs: Programmable Conditional Regulation of CRISPR/Cas Function in Bacterial and Mammalian Cells via Dynamic RNA Nanotechnology. ACS Cent. Sci. 2019, 5 (7), 1241-1249.
(27) Liu, Y.; Zhan, Y.; Chen, Z.; He, A.; Li, J.; Wu, H.; Liu, L.; Zhuang, C.; Lin, J.; Guo, X.; Zhang, Q.; Huang, W.; Cai, Z. Directing Cellular Information Flow via CRISPR Signal Conductors. Nat Methods 2016, 13, 938-944.
(28) Tang, W.; Hu, J. H.; Liu, D. R. Aptazyme-Embedded Guide RNAs Enable Ligand-Responsive Genome Editing and Transcriptional Activation. Nat Commun 2017, 8, 15939.
(29) Kundert, K.; Lucas, J. E.; Watters, K. E.; Fellmann, C.; Ng, A. H.; Heineike, B. M.; Fitzsimmons, C. M.; Oakes, B. L.; Qu, J.; Prasad, N.; Rosenberg, O. S.; Savage, D. F.; El-Samad, H.; Doudna, J. A.; Kortemme, T. Controlling CRISPR-Cas9 with Ligand-Activated and Ligand-Deactivated SgRNAs. Nat Commun 2019, 10 (1), 2127. https://doi.org/10.1038/s41467-019-09985-2.
(30) Lee, Y. J.; Hoynes-O'Connor, A.; Leong, M. C.; Moon, T. S. Programmable Control of Bacterial Gene Expression with the Combined CRISPR and Antisense RNA System. Nucleic Acids Res. 2016, 44 (5), 2462-2473.
(31) Ferry, Q. R. V.; Lyutova, R.; Fulga, T. A. Rational Design of Inducible CRISPR Guide RNAs for de Novo Assembly of Transcriptional Programs. Nat Commun 2017, 8, 2109.
(32) Wang, X.-W.; Hu, L.-F.; Hao, J.; Liao, L.-Q.; Chiu, Y.-T.; Shi, M.; Wang, Y. A MicroRNA-Inducible CRISPR-Cas9 Platform Serves as a MicroRNA Sensor and Cell-Type-Specific Genome Regulation Tool. Nat. Cell Biol. 2019, 21 (4), 522-530. https://doi.org/10.1038/s41556-019-0292-7.
(33) Siu, K.-H.; Chen, W. Riboregulated Toehold-Gated GRNA for Programmable CRISPR-Cas9 Function. Nat. Chem. Biol. 2019, 15 (3), 217-220. https://doi.org/10.1038/s41589-018-0186-1.
(34) Oesinghaus, L.; Simmel, F. C. Switching the Activity of Cas12a Using Guide RNA Strand Displacement Circuits. Nat. Commun. 2019, 10 (1), 1-11. https://doi.org/10.1038/s41467-019-09953-w.
(35) Chen, B.; Gilbert, L. A.; Cimini, B. A.; Schnitzbauer, J.; Zhang, W.; Li, G.-W.; Park, J.; Blackburn, E. H.; Weissman, J. S.; Qi, L. S.; Huang, B. Dynamic Imaging of Genomic Loci in Living Human Cells by an Optimized CRISPR/Cas System. Cell 2013, 155 (7), 1479-1491.
(36) Knight, S. C.; Tjian, R.; Doudna, J. A. Genomes in Focus: Development and Applications of CRISPR-Cas9 Imaging Technologies. Angew Chem Int Ed 2018, 57 (16), 4329-4337.
(37) Hilton, I. B.; D′Ippolito, A. M.; Vockley, C. M.; Thakore, P. I.; Crawford, G. E.; Reddy, T. E.; Gersbach, C. A. Epigenome Editing by a CRISPR-Cas9-Based Acetyltransferase Activates Genes from Promoters and Enhancers. Nat Biotechnol 2015, 33, 510.
(38) Gaudelli, N. M.; Komor, A. C.; Rees, H. A.; Packer, M. S.; Badran, A. H.; Bryson, D. I.; Liu, D. R. Programmable Base Editing of A$\cdot$T to G$\cdot$C in Genomic DNA without DNA Cleavage. Nature 2017, 551 (7681), 464-471. https://doi.org/10.1038/nature24644.
(39) Hochrein, L. M.; Li, H.; Pierce, N. A. High-Performance Allosteric Conditional Guide RNAs for Mammalian Cell-Selective Regulation of CRISPR/Cas. ACS Synth. Biol. 2021, 10 (5), 964-971. https://doi.org/10.1021/acssynbio.1c00037.
(40) Zadeh, J. N.; Steenberg, C. D.; Bois, J. S.; Wolfe, B. R.; Pierce, M. B.; Khan, A. R.; Dirks, R. M.; Pierce, N. A. NUPACK: Analysis and Design of Nucleic Acid Systems. J. Comput. Chem. 2011, 32 (1), 170-173. https://doi.org/10.1002/jcc.21596.
(41) Wolfe, B. R.; Porubsky, N. J.; Zadeh, J. N.; Dirks, R. M.; Pierce, N. A. Constrained Multistate Sequence Design for Nucleic Acid Reaction Pathway Engineering. J. Am. Chem. Soc. 2017, 139 (8), 3134-3144.
(42) Wang, D.; Zhang, C.; Wang, B.; Li, B.; Wang, Q.; Liu, D.; Wang, H.; Zhou, Y.; Shi, L.; Lan, F.; Wang, Y. Optimized CRISPR Guide RNA Design for Two High-Fidelity Cas9 Variants by Deep Learning. Nat Commun 2019, 10 (1), 4284. https://doi.org/10.1038/s41467-019-12281-8.
(43) Misteli, T.; Spector, D. L. Applications of the Green Fluorescent Protein in Cell Biology and Biotechnology. Nat Biotech 1997, 15 (10), 961-964.
(44) Tsien, R. Y. The Green Fluorescent Protein. Annu Rev Biochem 1998, 67 (1), 509-544. https://doi.org/10.1146/annurev.biochem.67.1.509.
(45) Zimmer, M. Green Fluorescent Protein (GFP): Applications, Structure, and Related Photophysical Behavior. Chem Rev 2002, 102 (3), 759-782. https://doi.org/10.1021/cr010142r.
(46) Gao, Z.; Herrera-Carrillo, E.; Berkhout, B. Delineation of the Exact Transcription Termination Signal for Type 3 Polymerase III. Mol. Ther. Nucleic Acids 2018, 10, 36-44.
(47) Kieft, J. S.; Rabe, J. L.; Chapman, E. G. New Hypotheses Derived from the Structure of a Flaviviral Xrn1-Resistant RNA: Conservation, Folding, and Host Adaptation. RNA Biol 2015, 12 (11), 1169-1177. https://doi.org/10.1080/15476286.2015.1094599.
(48) Chavez, A.; Scheiman, J.; Vora, S.; Pruitt, B. W.; Tuttle, M.; Iyer, E. P. R.; Lin, S.; Kiani, S.; Guzman, C. D.; Wiegand, D. J.; Ter-Ovanesyan, D.; Braff, J. L.; Davidsohn, N.; Housden, B. E.; Perrimon, N.; Weiss, R.; Aach, J.; Collins, J. J.; Church, G. M. Highly Efficient Cas9-Mediated Transcriptional Programming. Nat. Methods 2015, 12 (4), 326-328. https://doi.org/10.1038/nmeth.3312.
(49) Mali, P.; Aach, J.; Stranges, P. B.; Esvelt, K. M.; Moosburner, M.; Kosuri, S.; Yang, L.; Church, G. M. CAS9 Transcriptional Activators for Target Specificity Screening and Paired Nickases for Cooperative Genome Engineering. Nat. Biotechnol. 2013, 31, 833-838.
(50) Nissim, L.; Perli, S. D.; Fridkin, A.; Perez-Pinera, P.; Lu, T. K. Multiplexed and Programmable Regulation of Gene Networks with an Integrated RNA and CRISPR/Cas Toolkit in Human Cells. Mol. Cell 2014, 54 (4), 698-710.
(51) Shagin, D. A.; Barsova, E. V.; Yanushevich, Y. G.; Fradkov, A. F.; Lukyanov, K. A.; Labas, Y. A.; Semenova, T. N.; Ugalde, J. A.; Meyers, A.; Nunez, J. M.; Widder, E. A.; Lukyanov, S. A.; Matz, M. V. GFP-like Proteins as Ubiquitous Metazoan Superfamily: Evolution of Functional Features and Structural Complexity. Mol. Biol. Evol. 2004, 21 (5), 841-850. https://doi.org/10.1093/molbev/msh079.
(52) Nissim, L.; Perli, S. D.; Fridkin, A.; Perez-Pinera, P.; Lu, T. K. Multiplexed and Programmable Regulation of Gene Networks with an Integrated RNA and CRISPR/Cas Toolkit in Human Cells. Mol Cell 2019, 54 (4), 698-710.
(53) Zadeh, J. N.; Wolfe, B. R.; Pierce, N. A. Nucleic Acid Sequence Design via Efficient Ensemble Defect Optimization. J. Comput. Chem. 2011, 32, 439-452. https://doi.org/10.1002/jcc.21633.
(54) Wolfe, B. R.; Pierce, N. A. Sequence Design for a Test Tube of Interacting Nucleic Acid Strands. ACS Synth. Biol. 2015, 4 (10), 1086-1100.

What is claimed is:

1. A method of conditionally mediating the function of a Cas protein effector on a target gene, comprising: providing an allosteric conditional guide RNA (cgRNA) comprising a 5′ fragment (cg5) and a 3′ fragment (cg3), cg5 comprising a target-binding region and a trigger-binding region, and cg3 comprising a cognate RNA trigger:
a. wherein the target-binding region is non-overlapping with the trigger-binding region and does not bind either the target-binding region or 5′ of the target-binding region;
b. wherein fragment cg5 comprises a trigger-binding region comprising a 5′ portion of a stem of a terminator duplex; and fragment cg3 comprises a 3′ portion of the stem of the terminator duplex, such that hybridization of cg5 to cg3 forms the terminator duplex, activating the cgRNA;
c. wherein cg5 and cg3 are inactive when not bound to each other; and
d. wherein cg3 hybridizes to cg5 to form a cg5:cg3 complex, activating the cgRNA to mediate the function of a Cas protein effector on a target gene that binds the target-binding region.

2. The method of claim 1, wherein the fragment cg5 further comprises a Cas handle wherein the target-binding region is 5′ of the Cas handle and the trigger-binding region is 3′ of the Cas handle.

3. The method of claim 1, wherein the trigger is, or is a subsequence of an mRNA, an rRNA, a lncRNA, a miRNA, or a tRNA.

4. The method of claim 1, wherein the cgRNA is expressed in a cell.

5. The method of claim 1, wherein the cgRNA is chemically synthesized.

6. The method of claim 1, wherein the cgRNA further comprises one or more additional regions at the 5′ and/or the 3′ end.

7. The method of claim 1, wherein the cgRNA, and/or trigger comprises one or more chemical modifications that alter one or more of degradation properties, affinity, biological activity, and/or delivery properties of the cgRNA.

8. The method of claim 1, wherein the cgRNA, and/or trigger comprises one or more chemical modifications selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′O-Methyl (20-Me) modification, and 2′O-(2-methoxyethyl) (20-MOE) modification.

9. The method of claim 1, wherein the cgRNA, and/or trigger further comprises a protective element (PEL), wherein none, some, or all of the PEL sequence is derived from a component of a viral xrRNA sequence, and wherein the PEL reduces degradation of the RNA trigger, RNA helper, and/or RNA inhibitor in a prokaryotic or eukaryotic cell.

10. The method of claim 1, wherein the cgRNA further comprises a protective element (PEL), wherein none, some, or all of the PEL sequence is derived from a component of a viral xrRNA, and wherein the PEL reduces degradation of the cgRNA and/or the cgRNA fragments in a prokaryotic or eukaryotic cell.

11. The method of claim 1, wherein the cgRNA works in conjunction with Cas to mediate cell-selective induction, silencing, editing, or binding of a target gene.
